(19) United States
(12) Patent Application Publication
(10) Pub. No.: US 2025/0319069 A1
(43) Pub. Date: Oct. 16, 2025

(54) METHODS FOR TREATING DEPRESSIVE STATES

(71) Applicant: BioXcel Therapeutics, Inc., New Haven, CT (US)

(72) Inventors: Robert RISINGER, New Haven, CT (US); Charles BEASLEY, New Haven, CT (US); Michael DE VIVO, New Haven, CT (US); Frank D. YOCCA, New Haven, CT (US)

(21) Appl. No.: 18/574,404

(22) PCT Filed: Jul. 1, 2022
(86) PCT No.: PCT/US2022/035919

§ 371 Date: Dec. 27, 2023

Related U.S. Application Data
(60) Provisional application No. 63/218,031, filed on Jul. 2, 2021.
(60) Provisional application No. 63/247,122, filed on Sep. 22, 2021.

(51) Int. Cl. A61K 31/4174; A61K 31/137; A61K 31/165; A61K 31/343; A61K 31/381; A61K 31/5375; A61P 25/24

(52) U.S. Cl. CPC A61K 31/4174; A61K 31/137; A61K 31/165; A61K 31/343; A61K 31/381; A61K 31/5375; A61P 25/24

(57) ABSTRACT

The present disclosure relates to method of treating or preventing depression in a human subject having Major Depressive Disorder or a Major Depressive Episode, the method comprising (a) an induction phase, comprising administering to the subject a therapeutic amount of dexmedetomidine or a pharmaceutically acceptable salt thereof in combination with a therapeutic amount of a selective serotonin reuptake inhibitor (SSRI)/serotonin norepinephrine reuptake inhibitors (SNRI) from about 1 day to about 28 days, followed by (b) a maintenance phase comprising administering to the subject a therapeutic amount of the SSRI/SNRI.

### CROSS REFERENCE TO RELATED APPLICATIONS

This application claims the benefit of priority under 35 U.S.C. 119 (e) to U.S. Provisional Patent Application No. 63/218,031 on Jul. 2, 2021; U.S. Provisional Patent Application No. 63/247,122 on Sep. 22, 2021; the disclosures of each of which are incorporated herein by reference in their entireties.

### FIELD

The present disclosure relates to method of treating or preventing depression in a human subject in need thereof.

### BACKGROUND

Major depressive disorder is characterized by discrete episodes of at least 2 weeks' duration (although most episodes last considerably longer) involving clear-cut changes in affect, cognition, and neurovegetative functions and inter-episode remissions. This may also be accompanied by hypomanic or manic symptoms (i.e., fewer symptoms or for a shorter duration than required for mania or hypomania). Per the Diagnostic and Statistical Manual of Mental Disorders, 5th Edition (DSM-5), an individual must have at least one of the symptoms either (1) depressed mood or (2) loss of interest or pleasure. Research studies suggest that between 40% and 60% of patients with major depressive disorder (MDD) also have anxiety symptoms and share features relevant to anxiety that make them difficult to distinguish in practice. Specifically, both groups feature psychic agitation and inner tension that contribute to the manifestation of anxiety. Patients with anxiety disorders experience substantial physical and emotional discomfort and have elevated rates of substance use and medical illnesses. Co-occurring anxiety disorders in the context of other psychiatric disorders, for example major depressive disorder (MDD) or bipolar disorder, are associated with a more chronic and treatment refractory course and these patients are at an elevated risk for suicide.

The etiology of major depressive disorder is believed to be multifactorial, including biological, genetic, environmental, and psychosocial factors. MDD was earlier considered to be mainly due to abnormalities in neurotransmitters, especially serotonin, norepinephrine, and dopamine. People with suicidal ideations have been found to have low levels of serotonin metabolites. This has been evidenced by the use of different antidepressants such as selective serotonin receptor inhibitors, serotonin-norepinephrine receptor inhibitors, dopamine-norepinephrine receptor inhibitors for the treatment of depression. The treatment with selective serotonin reuptake inhibitors (SSRIs) and serotonin-norepinephrine reuptake inhibitors (SNRIs) provide relief by increasing serotonin and norepinephrine levels in the brain, In addition, antidepressants like SSRIs and SNRIs usually take about 4 to 6 weeks of dosing before the beneficial therapeutic effects become apparent side-effects occur that can lead to worse clinical outcomes including a higher risk of suicide, or termination of treatment before the beneficial therapeutic effects occur.

Additionally, not all patients treated with an SSRI or SNRI with a sufficient dose for a sufficient length of time to achieve a clinically meaningful benefit.

Thus, there exists a need to accelerate the time to response when a patient is treated with an SSRI or SNRI and reduce the burden of anxious and/or agitated symptoms that can be caused by the primary antidepressant (SSRI or SNRI).

There also exists a need to augment/enhance therapeutic response to an SSRI or SNRI in those patients, who in spite of an adequate therapeutic trial, fail to achieve a clinically meaningful improvement in their depressive symptoms SUMMARY

The present disclosure provides methods for administering dexmedetomidine or a pharmaceutically acceptable salt thereof alone or in combination with one or more SSRIs/SNRIs to either accelerate therapeutically beneficial outcomes in subjects with major depressive episodes in in the context of a diagnosis of either Major Depressive Disorder or Bipolar Disorder, or augment (i.e., improve) the response in a patient who has failed to show an adequate therapeutic response to an adequate course of treatment with an SSRI or SNRI.

The present disclosure also provides methods of treating major depressive disorder (MDD) in a subject in need thereof, comprising administering to the subject dexmedetomidine or a pharmaceutically acceptable salt thereof as a monotherapy, wherein the subject is non-agitated. In embodiments, the major depressive disorder is associated with anxious distress. In embodiments, the major depressive disorder is associated with or without a medical condition. In embodiments, the subject has an additional comorbidity or disorder such as obsessive-compulsive disorder, an anxiety disorder, social phobia, PTSD, panic disorder or generalized anxiety disorder. In embodiments, the major depressive disorder is mild or moderate. In embodiments, the major depressive disorder is severe.

In embodiments, the present disclosure provides methods of treating major depressive disorder (MDD) in a non-agitated subject in need thereof, comprising administering oromucosally (includes buccal, sublingual, gingival) about 10 micrograms to about 200 micrograms of dexmedetomidine or a pharmaceutically acceptable salt thereof to the subject. In embodiments, the present disclosure provides methods of treating major depressive disorder (MDD) in a non-agitated subject in need thereof, comprising administering oromucosally about 30 micrograms to about 200 micrograms of dexmedetomidine or a pharmaceutically acceptable salt thereof to the subject.

The present disclosure also provides methods of treating major depressive episodes (MDE) in a subject in need thereof, comprising administering to the subject dexmedetomidine or a pharmaceutically acceptable salt thereof as a monotherapy, wherein the subject is non-agitated.

In embodiments, the major depressive episode is associated with anxious distress. In embodiments, the major depressive episode is associated with or without a medical condition. In embodiments, the subject has an additional comorbidity or disorder such as obsessive-compulsive disorder, an anxiety disorder, social phobia, PTSD, panic disorder or generalized anxiety disorder. In embodiments, the subject has a bipolar disorder. In embodiments, the subject has a major depressive disorder. In embodiments, the subject does not have a bipolar disorder. In embodiments, the major depressive episode is mild or moderate. In embodiments, the major depressive episode is severe.

In embodiments, the present disclosure provides methods of treating major depressive episode (MDE) in a non-agitated subject in need thereof, comprising administering oromucosally (includes buccal, sublingual, gingival) about 10 micrograms to about 200 micrograms of dexmedetomidine or a pharmaceutically acceptable salt thereof to the subject. In embodiments, the present disclosure provides methods of treating major depressive episode (MDE) in a non-agitated subject in need thereof, comprising administering oromucosally about 30 micrograms to about 200 micrograms of dexmedetomidine or a pharmaceutically acceptable salt thereof to the subject.

In embodiments, dexmedetomidine or pharmaceutically acceptable salt thereof is administered oromucosally as a tablet, wafer, patch, film, gel or spray or liquid drops. In embodiments, dexmedetomidine or pharmaceutically acceptable salt thereof is administered oromucosally as a film.

In embodiments, dexmedetomidine or a pharmaceutically acceptable salt thereof is administered for an extended period or chronically such as over a period of weeks or months.

In embodiments, the present disclosure provides a method of reducing score on HAM-D scale in a human subject suffering from major depressive disorder, comprising administering oromucosally about 30 micrograms to about 90 micrograms of dexmedetomidine or a pharmaceutically acceptable salt thereof during daytime and about 90 micrograms to about 180 micrograms of dexmedetomidine or a pharmaceutically acceptable salt thereof during night-time, wherein the subject is non-agitated.

In embodiments, the present disclosure provides a method of reducing score on MADRS scale in a human subject suffering from major depressive disorder, comprising administering oromucosally about 30 micrograms to about 90 micrograms of dexmedetomidine or a pharmaceutically acceptable salt thereof during daytime and about 90 micrograms to about 180 micrograms of dexmedetomidine or a pharmaceutically acceptable salt thereof during night-time, wherein the subject is non-agitated.

In embodiments, the present disclosure provides a method of reducing score on HAM-D scale in a human subject suffering from major depressive episode, comprising administering oromucosally about 30 micrograms to about 90 micrograms of dexmedetomidine or a pharmaceutically acceptable salt thereof during daytime and about 90 micrograms to about 180 micrograms of dexmedetomidine or a pharmaceutically acceptable salt thereof during night-time, wherein the subject is non-agitated.

In embodiments, the present disclosure provides a method of reducing score on MADRS scale in a human subject suffering from major depressive episode, comprising administering oromucosally about 30 micrograms to about 90 micrograms of dexmedetomidine or a pharmaceutically acceptable salt thereof during daytime and about 90 micrograms to about 180 micrograms of dexmedetomidine or a pharmaceutically acceptable salt thereof during night-time, wherein the subject is non-agitated.

In embodiments, the present disclosure provides the use of dexmedetomidine or a pharmaceutically acceptable salt thereof as an adjunctive therapy to one or more conventional anti-depressants in subjects with major depressive disorder (MDD).

In embodiments, the present disclosure provides the use of dexmedetomidine or a pharmaceutically acceptable salt thereof as an adjunctive therapy to one or more conventional anti-depressants in subjects with major depressive episode (MDE).

In embodiments, the present disclosure provides the use of dexmedetomidine or a pharmaceutically acceptable salt thereof as an adjunctive therapy to one or more conventional anti-depressants in subjects with anxious distress.

In embodiments, the present disclosure provides methods of accelerating the anti-depressant response in a subject with major depressive disorder (MDD), comprising administering dexmedetomidine or a pharmaceutically acceptable salt thereof in combination with one or more conventional anti-depressants (e.g. SSRI/SNRIs) to the subject.

In embodiments, the present disclosure provides methods of accelerating the anti-depressant response in a subject with major depressive episode (MDE), comprising administering dexmedetomidine or a pharmaceutically acceptable salt thereof in combination with one or more conventional anti-depressants (e.g. SSRI/SNRIs) to the subject.

The present disclosure also provides methods of treating a major depressive episode in the context of a diagnosis of either Major Depressive Disorder or Bipolar Disorder to augment (i.e., improve) the therapeutic response to an SSRI or SNRI in a patient in need thereof, comprising administering to the patient dexmedetomidine or a pharmaceutically acceptable salt thereof as co-therapy along with an ongoing and continued treatment SSRI or an SNRI, whether or not the patient experiences anxiety- and/or agitation-related symptoms at the time of initiation of treatment.

The present disclosure also provides methods of treating a major depressive episode in the context of a diagnosis of either Major Depressive Disorder or Bipolar Disorder to accelerate the therapeutic response to an SSRI in a patient in need thereof, comprising administering to the patient dexmedetomidine or a pharmaceutically acceptable salt thereof as co-therapy along with an SSRI or an SNRI, whether or not the patient experiences anxiety- and/or agitation-related symptoms at the time of initiation of treatment in a patient in need thereof.

In embodiments, the conventional antidepressant is selected from the group consisting of, but not limited to, selective serotonin reuptake inhibitors (SSRIs); selective serotonin and norepinephrine reuptake inhibitors (SNRIs); older tricyclic antidepressants (TCAs); monoamine oxidase inhibitors (MAO-inhibitors), reversible inhibitors of monoamine oxidase (RIMAs), atypical antidepressants; tertiary amine tricyclics and secondary amine tricyclic antidepressants. In embodiments, the conventional antidepressant is an SSRI or an SNRI. In embodiments, the subject has failed or inadequate response to current anti-depressant therapy. In embodiments, the subject has no previous exposure of any conventional anti-depressant therapy.

In embodiments, the subject also has anxious distress.

In embodiments, dexmedetomidine or a pharmaceutically acceptable salt thereof is administered immediately the subject is started on conventional antidepressant therapy to provide acceleration of the antidepressant response and provide interim treatment for the period of about 1 to 4 weeks while the conventional antidepressant becomes effective and side effects subside. In embodiments, dexmedetomidine or a pharmaceutically acceptable salt thereof is administered as “rescue” medication in subjects who may still suffer from an episode of a symptom of the major depressive disorder (such as an episode of depressed mood) even while on conventional antidepressant therapy, to relieve and palliate the symptoms of that episode as needed. In embodiments, dexmedetomidine or a pharmaceutically acceptable salt thereof is administered in a scheduled fashion together with conventional antidepressant therapy as a part of a complete antidepressant regimen.

In embodiments, the present disclosure provides a method of reducing score on HAM-D scale in a human subject suffering from major depressive disorder, comprising administering an oromucosally therapeutic amount of dexmedetomidine or a pharmaceutically acceptable salt thereof in combination with an orally therapeutic amount of an SSRI/SNRI.

In embodiments, the present disclosure provides a method of reducing score on HAM-D scale in a human subject suffering from major depressive episode, comprising administering an oromucosally therapeutic amount of dexmedetomidine or a pharmaceutically acceptable salt thereof in combination with an orally therapeutic amount of an SSRI/SNRI.

Patients typically present with different baseline HAM-D scores, and higher scores associated with more severe depression. HAM-D scores below 7 indicate no depression. Mild depression ranges from 7-17 points, moderate depression from 18-24 points and severe depression is over 25 points.

In embodiments, the subject has a HAM-D-17 total score ≥18 at the start of treatment (or baseline). In embodiments, the subject has a HAM-D-17 total score >14 at the start of the treatment.

In embodiments, the methods described herein reduce the HAM-D score by about 5% and about 100%, for example, about 5%, about 10%, about 15%, about 20%, about 25%, about 30%, about 35%, about 40%, about 45%, about 50%, about 55%, or more, compared to baseline score observed prior to treatment.

In aspects, the HAM-D score may be lowered to 5, 6, 7, 8, or 9 points; preferably, the HAM-D score is lowered below 7 or to 7. Advantageously, administering dexmedetomidine and anti-depressants as disclosed herein achieves the reduced HAM-D scores faster than using an anti-depressant alone, providing substantially improved adoption of therapy and reducing therapy cessation. The lowered HAM-D scores may be achieved within 4 weeks of initiating therapy, preferably within 1 to 2 weeks. This is in contrast to patients on anti-depressants alone, who typically take far longer to achieve HAM-D scores in the 5 to 9 range or to see decreases of 50% in their HAM-D scores. Patients suffering from moderate or severe depression may not achieve such low scores after treatment, but may achieve a drop in points that provides significant relief of their depression; for example, the drop may be about 4 points to about 8 points. In embodiments, the subject has a MADRS score ≥20 at the start of treatment (or baseline score). In embodiments, the subject has a MADRS score ≥10 at the start of the treatment.

In embodiments, the present disclosure provides a method of reducing score on MADRS scale in a human subject suffering from major depressive disorder, comprising administering an oromucosally therapeutic amount of dexmedetomidine or a pharmaceutically acceptable salt thereof in combination with an orally therapeutic amount of an SSRI/SNRI, In embodiments, the present disclosure provides a method of reducing score on MADRS scale in a human subject suffering from major depressive episode, comprising administering an oromucosally therapeutic amount of dexmedetomidine or a pharmaceutically acceptable salt thereof in combination with an orally therapeutic amount of an SSRI/SNRI.

In embodiments, the methods described herein provide the reduction in MADRS score of about 5% and about 100%, for example, about 5%, about 10%, about 15%, about 20%, about 25%, about 30%, about 35%, about 40%, about 45%, about 50%, about 55%, or more, compared to baseline score observed prior to treatment.

In embodiments, the present disclosure provides a method of treating or preventing anxious distress in a human subject, the method comprising: (a) an induction phase, comprising administering to the subject a therapeutic amount of dexmedetomidine or a pharmaceutically acceptable salt thereof in combination with a therapeutic amount of an SSRI/SNRI from about 1 day to about 28 days, followed by (b) a maintenance phase comprising administering to the subject a therapeutic amount of the SSRI/SNRI.

In embodiments, the induction phase comprising a treatment period of at least about 1 to 4 weeks.

In embodiments, the present disclosure provides a method of treating or preventing anxious distress caused by beginning treatment with a SSRI or SNRI in a human subject, comprising: (a) an induction phase, comprising administering to the subject a therapeutic amount of dexmedetomidine or a pharmaceutically acceptable salt thereof in combination with a therapeutic amount of an SSRI/SNRI from about 1 day to about 28 days, followed by (b) a maintenance phase comprising administering to the subject a therapeutic amount of the SSRI/SNRI.

In embodiments, the SSRI/SNRI in the maintenance phase is administered as a monotherapy. In embodiments, the SSRI/SNRI in the maintenance phase is administered with an additional SSRI/SNRI. In embodiments, the maintenance phase is continued until the underlying disease (e.g. major depressive disorder) resolves. In embodiments, the maintenance phase is continued until further treatment is not required (as determined by a clinician or physician). In embodiments, the maintenance phase is continued until the subject experiences a recurrence of anxious distress. In embodiments, the subject is in a manic phase, a depressed phase or both. In embodiments, the subject has undergone previous treatment with SSRIs/SNRIs (e.g., for at least one week) prior to the induction phase. In embodiments, the subject has undergone previous treatment with SSRIs/SNRIs (e.g., for at least two or three weeks) prior to the induction phase.

In embodiments, when a subject experiences a recurrence of anxious distress then they may begin a second induction phase comprising administering a therapeutic amount of dexmedetomidine or a pharmaceutically acceptable salt thereof in combination with a therapeutic amount of a SSRI/SNRI from about 1 day to about 28 days, followed by a maintenance phase, comprising administering to the subject a therapeutic amount of the SSRI/SNRI. In embodiments, the maintenance phase is continued for at least 4 weeks. In embodiments, the maintenance phase is continued for at least 6 weeks. In embodiments, the maintenance phase is continued for at least 8 weeks. In embodiments, the maintenance phase is continued for as long as needed for treatment.

In embodiments, dexmedetomidine or a pharmaceutically acceptable salt thereof and the SSRI/SNRI are administered in separate unit dosage forms. In embodiments, the unit dosage forms are co-administered for about 28 days, about 27 days, about 26 days, about 25 days, about 24 days, about 23 days, about 22 days, about 21 days, about 20 days, about 19 days, about 18 days, about 17 days, about 16 days, about 15 days, about 14 days, about 13 days, about 12 days, about 11 days, about 10 days, about 9 days, about 8 days, about 7 days, about 6 days, about 5 days, about 4 days, about 3 days, about 2 days, or about 1 day. In embodiments, the unit dosage forms are co-administered for about 28 days. In embodiments, the unit dosage forms are co-administered for about 21 days. In embodiments, the unit dosage forms are co-administered for about 14 days. In embodiments, the unit dosage forms are co-administered for about 7 days. In embodiments, dexmedetomidine or a pharmaceutically acceptable salt thereof and the SSRI/SNRI are administered sequentially or simultaneously.

In embodiments, both unit dosage forms are administered separately by the same or different routes selected from the group consisting of oromucosal (e.g., sublingual or buccal or gingival), oral, topical, transdermal, intranasal, parenteral (e.g., intravenous, intramuscular, subcutaneous) routes and the like. In embodiments, the dexmedetomidine or a pharmaceutically acceptable salt thereof and/or the SS/SNRI are administered in a dosage form selected from the group comprising tablets, orally disintegrating tablets (ODTs), effervescent tablets, capsules, pellets, pills, lozenges or troches, film, powders, dispersible granules, catchets, aqueous solutions, syrups, emulsions, suspensions, solutions, soft gels, dispersions and the like. In embodiments, dexmedetomidine or a pharmaceutically acceptable salt thereof (e.g. dexmedetomidine hydrochloride) is administered oromucosally in a dosage form selected from tablet, film, spray, gel or drops. In embodiments, the oromucosal dosage form comprising dexmedetomidine or a pharmaceutically acceptable salt thereof is a film. In embodiments, the SSRI/SNRI is administered orally as a tablet. In embodiments, the SSRI/SNRI is administered orally as a capsule. In embodiments, the SSRI/SNRI is administered orally as a delayed release capsule.

In embodiments, the SSRI/SNRI and dexmedetomidine or a pharmaceutically acceptable salt are administered oromucosally in a dosage form selected from tablet, film, spray, gel or drops, preferably film. In embodiments, dexmedetomidine or a pharmaceutically acceptable salt thereof (e.g. dexmedetomidine hydrochloride) and the SSRI/SNRI are administered together in a single unit dosage form (e.g. a film). In embodiments, dexmedetomidine and the SSRI/SNRI are present together in a single oral dosage form. Such unit dosage forms may contain dexmedetomidine and SSRI/SNRI as a homogenous mixture or in separate compartments of the unit dosage form.

In embodiments, oromucosal dosage form comprising dexmedetomidine or a pharmaceutically acceptable salt thereof is administered twice a day; for e.g. one in the morning and another in the evening; preferably at the same time daily. The evening dose may be taken within 1 to 2 hours, optionally 1 hour of the subject going to bed.

In embodiments, the subject has a total Score of ≥14 on the Hamilton Anxiety Scale (HAM-A) at the start of treatment (or baseline).

In embodiments, the present disclosure relates to an individual unit dosage form provided as a kit comprising the composition as described herein in a container with or without instructions for administration to a subject in need thereof. In embodiments, the present disclosure relates to two-unit dosage form provided as a kit comprising the composition as described herein in one or more containers with or without instructions for the simultaneous, sequential or separate administration to a subject in need thereof.

### BRIEF DESCRIPTION OF THE DRAWINGS

FIG. 1a: depicts the effect of treatment of Dexmedetomidine (10 μg/kg; i.p.), Fluoxetine (32 mg/kg; i.p.) and the combination of Dexmedetomidine (10 μg/kg; i.p.) with Fluoxetine (32 mg/kg; i.p.) in rats on time spent in the open arm in Elevated plus maze according to Example 1 (data represents Mean±SEM, One way ANOVA followed by Tukey's Multiple Comparison Test, ****p<0.0001 vs Vehicle, N=10 rats. CLZ: clobazam; DEX: dexmedetomidine; FLX: fluoxetine; i.p.: intraperitoneal).

FIG. 1b: depicts the effect of treatment of Dexmedetomidine (10 μg/kg; i.p.), Fluoxetine (32 mg/kg; i.p.) and the combination of Dexmedetomidine (10 μg/kg; i.p.) with Fluoxetine (32 mg/kg; i.p.) in rats on percentage of entries in the open arm in Elevated plus maze according to Example 1 (data represents Mean±SEM, One way ANOVA followed by Tukey's Multiple Comparison Test, **p<0.001 vs Vehicle, N=10 rats. CLZ: clobazam; DEX: dexmedetomidine; FLX: fluoxetine; i.p.: intraperitoneal).

FIG. 2: depicts the effect of treatment of Dexmedetomidine (10 μg/kg; i.p.), Fluoxetine (32 mg/kg; i.p.) and the combination of Dexmedetomidine (10 μg/kg; i.p.) with Fluoxetine (32 mg/kg; i.p.) in rats on time spent in the open arm in Elevated plus maze according to Example 1 (data represents Mean±SEM, One way ANOVA followed by Tukey's Multiple Comparison Test, ****p<0.0001 vs Vehicle, N=10 rats. CLZ: clobazam; DEX: dexmedetomidine; FLX: fluoxetine; i.p.: intraperitoneal).

FIG. 3: depicts the effect of treatment of Dexmedetomidine (10 μg/kg; i.p.), Fluoxetine (32 mg/kg; i.p.) and the combination of Dexmedetomidine (10 μg/kg; i.p.) with Fluoxetine (32 mg/kg; i.p.) in rats on total number of entries in the four arms of the Elevated plus maze according to Example 1 (data represents Mean±SEM, One way ANOVA followed by Tukey's Multiple Comparison Test, *p<0.05 vs Vehicle, N=10 rats. CLZ: clobazam; DEX: dexmedetomidine; FLX: fluoxetine; i.p.: intraperitoneal).

FIG. 4: depicts the effect of treatment of Dexmedetomidine (10 μg/kg; i.p.), Fluoxetine (32 mg/kg; i.p.) and the combination of Dexmedetomidine (10 μg/kg; i.p.) with Fluoxetine (32 mg/kg; i.p.) in rats on total distance travelled in the four arms of the Elevated plus maze according to Example 1 (data represents Mean±SEM, One way ANOVA followed by Tukey's Multiple Comparison Test, *p<0.05 vs Vehicle; **p<0.01 vs Vehicle, N=10 rats. CLZ: clobazam; DEX: dexmedetomidine; FLX: fluoxetine; i.p.: intraperitoneal).

FIG. 5: depicts the effect of treatment of Dexmedetomidine (10 μg/kg; i.p.), Fluvoxamine (3 and 10 mg/kg; i.p.) and the combination of Dexmedetomidine (10 μg/kg; i.p.) with Fluvoxamine (3 and 10 mg/kg; i.p.) in rats with Yohimbine-induced anxiety on number of entries in the open arm in Elevated plus maze according to Example 2 (data represents Mean±SEM, One way ANOVA followed by Tukey's Multiple Comparison Test, ^{$$$$}p<0.0001 vs YOH (2.5 mg/kg, i.p.), *p<0.05 vs YOH (2.5 mg/kg; i.p.)+DEX (10 μg/kg; i.p.), **p<0.01 vs YOH (2.5 mg/kg; i.p.)+DEX (10 μg/kg; i.p.), \hat{ }\hat{ }p<0.01 vs YOH (2.5 mg/kg; i.p.)+FLUVO (3 mg/kg; i.p.), N=12 rats. DEX: dexmedetomidine; FLUVO: fluvoxamine; i.p.: intraperitoneal; YOH: yohimbine).

FIG. 6: depicts the effect of treatment of Dexmedetomidine (10 μg/kg; i.p.), Fluvoxamine (3 and 10 mg/kg; i.p.) and the combination of Dexmedetomidine (10 μg/kg; i.p.) with Fluvoxamine (3 and 10 mg/kg; i.p.) in rats with Yohimbine-induced anxiety on time spent in the open arm in Elevated plus maze according to Example 2 (data represents Mean±SEM, One way ANOVA followed by Tukey's Multiple Comparison Test, ^{$$$$}p<0.0001 vs YOH (2.5 mg/kg, i.p.), ^{$$}p<0.01 vs YOH (2.5 mg/kg, i.p.), **p<0.01 vs YOH (2.5 mg/kg; i.p.)+DEX (10 μg/kg; i.p.), ***p<0.001 vs YOH (2.5 mg/kg; i.p.)+DEX (10 μg/kg; i.p.), \hat{ }\hat{ }\hat{ }\hat{ }p<0.0001 vs YOH (2.5 mg/kg; i.p.)+FLUVO (3 mg/kg; i.p.), N=12 rats. DEX: dexmedetomidine; FLUVO: fluvoxamine; i.p.: intraperitoneal; YOH: yohimbine).

### DETAILED DESCRIPTION

In the following passages, different aspects of the disclosure are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

### Abbreviations

As used herein, the following abbreviations have the following meanings:
AE: adverse events
ACES: Agitation-Calmness Evaluation Scale
ADS: anxious distress specifier
BOLD: blood oxygen level-dependent
CGI: clinical global impression scale
CGI-I: clinical global Impressions scale—Improvement
CGI-S: clinical global Impressions scale—severity
DSM-5: Diagnostic and Statistical Manual of Mental Disorders, Fifth Edition
DEX: Dexmedetomidine
CMAI: Cohen-Mansfield Agitation Inventory
C-SSRS: Columbia-Suicide Severity Rating Scale
DASS: Depression and Anxiety Stress Scale
ECG: Electro cardiography
HAM-A: Hamilton anxiety rating scale
HDRS or (HAM-D): Hamilton Depression Rating Scale
HPMC: hydroxypropyl methylcellulose
ICF: informed consent form
I.P.: intraperitoneal
MAO: monoamine oxidase
MADRS: Montgomery-Asberg Depression Rating Scale
MED DRA—Medical Dictionary for Regulatory Activities
MDD: major depressive disorder
MDE: major depressive episodes
MINI: Mini-International Neuropsychiatric Interview
mg: milligrams
NPI-C: Neuropsychiatric Inventory-Clinician Rating Scale
NS: Not significant
PANSS or PEC: Positive and Negative Syndrome Scale
PBS: phosphate buffer saline
PK: Pharmacokinetics
QIDS-SR: Quick Inventory of Depressive Symptomatology
SL: sublingual
SNRI: serotonin-norepinephrine reuptake inhibitor
SSRI: selective serotonin reuptake inhibitor
STAI: State Inventory of the State Trait Anxiety Inventory
TEAE: treatment emergent adverse effects
UDS: urine drug screening
UTI: urinary tract infection
μg: micrograms

### Definitions

It will be understood that the terminology used herein is for the purpose of describing embodiments only and is not intended to be limiting. As used in this specification, the singular forms “a”, “an” and “the” include plural referents unless the context clearly dictates otherwise.

As used herein the term “about” or “approximately,” when used in connection with a numerical variable, generally refers to the value of the variable and to all values of the variable that are within the experimental error (e.g., within the 95% confidence interval for the mean) or ±10% of the indicated numerical value whichever is greater.

The terms “formulation” and “composition” are used interchangeably, except where otherwise clearly intended to have different meanings.

Throughout the present specification, numerical ranges are provided for certain quantities. It is to be understood that these ranges comprise all subranges therein. Thus, the range “from 50 to 80” includes all possible ranges therein (e.g., 51-79, 52-78, 53-77, 54-76, 55-75, 60-70, etc.). Furthermore, all values within a given range may be an endpoint for the range encompassed thereby (e.g., the range 50-80 includes the ranges with endpoints such as 55-80, 50-75, etc.).

The terms “comprises”, “comprising”, “includes”, “including”, “having” means “including but not limited to”.

As used herein, the terms “patient” or “subject” refers to a living organism suffering from or prone to a condition that can be treated by administration of a pharmaceutical composition as provided herein. Non-limiting examples include humans, other mammals and other non-mammalian animals.

As used herein, the term “therapeutic amount” or an “effective amount” is interchangeable with “therapeutically effective dose,” or “therapeutically effective amount” and refers to an amount sufficient to produce the desired effect or cause an improvement in a clinically significant condition of the subject.

As used herein, “pharmaceutically acceptable salt” refers to a salt known to be non-toxic and commonly used in the pharmaceutical literature. Typical inorganic acids used to form such salts include hydrochloric, hydrobromic, hydroiodic, nitric, sulfuric, phosphoric, hypophosphoric, and the like. Salts derived from organic acids, such as aliphatic mono and dicarboxylic acids, phenyl substituted alkanoic acids, hydroxyalkanoic and hydroxyl alkandioic acids, aromatic acids, aliphatic and aromatic sulfonic acids may also be used.

The terms “treat”, “treating” or “treatment” in reference to a particular disease or disorder includes lessening, improving, ameliorating or abrogating the symptoms and/or pathology of the disease or disorder. Treatment may be measured as a reduction level by at least 10% or higher, preferably 20% or higher, more preferably 40% or higher, even more preferably 60%.

The term “prevention” means preventing the occurrence of a disease, condition, or associated symptoms or preventing the recurrence of the same, for example after a period of improvement.

The term “adjunctive therapy” as used herein means the addition or combination of dexmedetomidine with one or more conventional antidepressants in order to enhance the efficacy of the conventional antidepressant and/or allow lower doses of the conventional antidepressant thus reducing side effects in treating or preventing the reoccurrence of major depressive disorder or major depressive episode or anxious distress.

The term “selective serotonin reuptake inhibitor” or “SSRI” means an inhibitor of the monoamine transporters which has stronger inhibitory effect at the serotonin transporter than the dopamine and the noradrenaline transporters.

The term “selective norepinephrine reuptake inhibitor” or “SNRI” means monoamine reuptake inhibitors that inhibit the reuptake of serotonin and noradrenaline, both neurotransmitters are thought to play an important role in mood regulation.

The term “major depressive episode” or “MDE” as per DSM-5 refers to a period of at least two weeks when a person experienced a depressed mood or loss of interest or pleasure in daily activities, and had a majority of specified symptoms, such as problems with sleep, eating, energy, concentration, or self-worth. During these episodes, there is a deterioration in the patient's level of function or symptoms must cause the patient considerable distress. Episodes may be isolated or recurrent. To diagnose a major depressive episode, a healthcare provider must make sure that:
the symptoms do not meet the criteria for a mixed episode.
The symptoms must cause considerable distress or impair functioning at work, in social settings or in other important areas in order to qualify as an episode.
The symptoms are not due to the direct physiological effects of a substance (e.g., abuse of a drug or medication) or a general medical condition (e.g., hypothyroidism)

The term “MDD” or “Major Depressive Disorder” refers to presence of two main depressive symptoms: a depressed mood, and loss of interest/pleasure in activities (anhedonia) as well as five out of the nine more specific symptoms (as listed below), and must cause the patient considerable distress or result in a significant deterioration in the patient's level of function, must frequently occur for more than two weeks (to the extent in which it impairs functioning) for the diagnosis. 1) Depressed mood most of the day, nearly every day as indicated by either subjective report (e.g., feels sad, empty, hopeless) or observation made by others (e.g., appears tearful). 2) Markedly diminished interest or pleasure in all, or almost all, activities most of the day, nearly every day. 3) Significant weight loss when not dieting or weight gain (e.g., a change of more than 5% of body weight in a month), or decrease or increase in appetite nearly every day. 4) Insomnia or hypersomnia nearly every day. 5) Psychomotor agitation or retardation nearly every day. 6) Fatigue or loss of energy nearly every day. 7) Feelings of worthlessness or excessive or inappropriate guilt nearly every day. 8) Diminished ability to think or concentrate, or indecisiveness, nearly every day. 9) Recurrent thoughts of death (not just fear of dying), recurrent suicidal ideation without a specific plan, or a suicide attempt or a specific plan for committing suicide. Major depressive episodes are characteristic of occur in the both the diagnoses of Major Depressive Disorder and Bipolar disorder. Diagnostic criteria rely on features, of course—namely, the presence or absence of manic or hypomanic episodes—to distinguish between the two diagnoses. In some cases, however, a history of mood elevation is underreported by patients; in others, patients who appear to be in a depressive episode simply have not yet experienced a manic episode.

The term “Bipolar disorder” refers to a condition that causes an individual to experience unusual shifts in mood, energy, activity levels, and the ability to carry out day-to-day tasks. Bipolar disorder includes bipolar I disorder, bipolar II disorder, and cyclothymic disorder. Bipolar I disorder is defined by manic episodes that last at least 7 days or by severe manic symptoms that require hospitalization. A subject with bipolar I disorder may also experience depressive episodes typically lasting at least 2 weeks. Episodes of depression with mixed features i.e. depressive and manic symptoms at the same time, are also possible. Bipolar II disorder is characterized by a pattern of depressive and hypomanic episodes, but not severe manic episodes typical of bipolar I disorder. Cyclothymic disorder (also referred to as cyclothymia) is characterized by periods of hypomanic symptoms (elevated mood and euphoria) and depressive symptoms lasting over a period of at least 2 years.

The term “anxiety” refers to an unpleasant state that involves a complex combination of emotions that include fear, apprehension, and worry. It is often accompanied by physical sensations such as heart palpitations, nausea, chest pain, shortness of breath, or tension headache.

The term “anxious distress” or “ADS” means high levels of anxiety or co-morbid/concurrent anxiety disorders in subjects with major depressive disorder, major depressive episodes, or bipolar disorder. To meet the criteria of DSM-5 anxious distress specifier, the subject must exhibit two or more of the following symptoms: (1) feeling keyed up or tense; (2) feeling unusually restless; (3) difficulty concentrating because of worry; (4) fear that something awful may happen; and (5) feeling that the individual might lose control of himself or herself.

The term “agitation”, as used herein, means a disorder characterized by symptoms of irritability, emotional outburst, impaired thinking, or excess motor and verbal activity that may occur due to either dysfunction of specific brain regions such as frontal lobes or due to dysfunction of neurotransmitter systems such as the noradrenergic system. In embodiments, the agitation may be caused by noradrenergic hyperarousal. In the present disclosure, an agitated subject may also exhibit aggression. The agitation may be acute or chronic. The agitation may be severe.

As used herein, the terms “pharmaceutically acceptable excipient” and “pharmaceutically acceptable carrier” refer to a substance that aids the administration of an active agent to and absorption by a subject. It also refers to an excipient that can be included in the compositions or formulations of the disclosure and that causes no significant adverse toxicological effect on the patient.

The term “unit dosage form” refers to physically discrete units suitable as unitary dosages for human subjects and other mammals (e.g., dogs), each unit containing a predetermined quantity of active material calculated to produce the desired onset, tolerability, and/or therapeutic effects, in association with a suitable pharmaceutical excipient (e.g., an ampoule).

The term “oromucosal” means administration to the oral mucosa, specifically the oral cavity and/or the pharynx. Oromucosal administration includes sublingual, buccal or gingival routes.

The term “sublingual” means “under the tongue” and refers to a method of administering substances via the blood vessels under the tongue rather than via the digestive tract. Sublingual absorption occurs through the highly vascularized sublingual mucosa, which allows a substance direct access to the blood circulation, thereby providing for direct systemic administration independent of gastrointestinal influences and avoiding undesirable first-pass hepatic metabolism.

The term “buccal” means administration of the dosage form against the gum and the inner lip or cheek.

The term “gingival” means administration of the dosage form to the gingiva (gums) found in the oral cavity of humans surrounding part of their teeth.

The term “film” herein includes films comprising polymers in any shape, including rectangular, square, or other desired shape. The film may be of any desired thickness and size, such that it can be conveniently placed oromucosally in the patient. For example, the film may be a thin film having a thickness of from about 20 micrometers to about 200 micrometers or may be a thick film having a thickness of from about 201 micrometers to about 1000 micrometers. In embodiments, the film may have a thickness greater than about 30 micrometers. In embodiments, the film is self-supporting. The term “self-supporting” means the films herein maintain structural integrity upon handling without the need for a backing layer. Some flexibility in the film is contemplated and may be desirable.

The term “mucoadhesion” is used herein to refer to adhesion to mucosal membranes, such as those in the oral cavity.

The term “mucoadhesive” refers to the property of adhering to a mucosal tissue surface in vivo. Such adhesion adherently localizes the dosage form onto the mucus membrane and requires the application of a force to separate the mucoadhesive material from the mucus membrane.

“Therapeutic” as used herein, may mean treatment and/or prophylaxis, depending on context.

The term “intranasal administration” means administration by the nasal route. The delivered drug can may have local or systemic effects.

The term “parenteral” refers to administration of a drug by injection under one or more layers of skin or mucous membrane, and can include, for example, subcutaneous, intravenous, intraperitoneal or intramuscular injection.

Within the meaning of the present disclosure, the term “co-administration” is used to refer to administration of dexmedetomidine or a pharmaceutically acceptable salt thereof and SSRIs/SNRIs or a pharmaceutically acceptable salt or ester or enantiomers thereof simultaneously or sequentially.

As used herein, “sequential administration” means that two compositions being administered to a subject are administered separated by a time interval sufficient to permit the resultant beneficial effect obtained when each composition exerts its effect.

The term “sequential administration” means that compositions are administered with a time separation of more than about 60 minutes; for example, 2 hours apart, 3 hours apart, 4 hours apart, 5 hours apart, 6 hours apart, 7 hours apart, 8 hours apart, 9 hours apart, 10 hours apart, 11 hours apart, or 12 hours apart. In embodiments, the compositions may be administered from about 1 hour to about 24 hours apart.

As used herein, “simultaneous administration” means at the same time or within a short period of time, for example, less than 1 hour, less than 30 minutes, less than 15 minutes, or less than 5 minutes.

### Active Agents:

### Dexmedetomidine:

Dexmedetomidine has the IUPAC name (+) 4-(S)-[1-(2,3-dimethylphenyl) ethyl]-1H-imidazole. As the monohydrochloride salt, it is predominantly used as a medication for the sedation of patients during treatment in an intensive care setting or to sedate patients prior to and/or during surgical and other procedures. Such medication is currently sold under the registered trade name “PRECEDEX®”.

Pharmaceutically acceptable salts of dexmedetomidine that may be used herein include generally any suitable salt that has been or may be approved by the US FDA or other appropriate foreign or domestic agency for administration to a human. Non-limiting examples of suitable pharmaceutically acceptable salts include salts of inorganic acids such as hydrochloric, hydrobromic, nitric, carbonic, monohydrocarbonic, phosphoric, monohydrogen phosphoric, dihydrogen phosphoric, sulfuric, hydrogen sulfuric, and hydroiodic acid. Other examples include salts derived from non-toxic organic acids, including acetic, propionic, isobutyric, maleic, malonic, benzoic, succinic, suberic, fumaric, lactic, mandelic, phthalic, benzenesulfonic, p-toluenesulfonic, citric, tartaric, and methanesulfonic acids, or combinations of these acid salts. Exemplary salts include dexmedetomidine hydrochloride, dexmedetomidine hydrobromide, dexmedetomidine sulfate, dexmedetomidine sulfonate, dexmedetomidine phosphate, dexmedetomidine nitrate, dexmedetomidine formate, dexmedetomidine citrate, dexmedetomidine tartrate, dexmedetomidine malate, dexmedetomidine benzoate, dexmedetomidine salicylate, dexmedetomidine ascorbate or the like. In embodiments, deuterated forms of dexmedetomidine or a pharmaceutically acceptable salt thereof may be included.

### Selective Serotonin Reuptake Inhibitors (SSRIs):

Selective serotonin reuptake inhibitors (SSRIs) are a class of drugs that boost serotonin levels and are typically used as antidepressants in the treatment of major depressive disorder, anxiety disorders, and other psychological conditions. These drugs are effective, well tolerated and have a favourable safety profile with varying degrees of selectivity for the other monoamine transporters, having little binding affinity for the noradrenaline and dopamine transporters. Exemplary selective serotonin reuptake inhibitors (SSRI) include, but are not limited to sertraline, fluoxetine, norfluoxetine, fluvoxamine, citalopram, escitalopram, dapoxetine, paroxetine, femoxetine, vortioxetine, alaproclate and vilazodone or a pharmaceutically acceptable salt or ester or enantiomer thereof. In embodiments, the SSRIs of the present disclosure have no effect on norepinephrine levels.

In embodiments, the SSRI is sertraline. Sertraline has the IUPAC name (1S,4S)-4-(3,4-dichlorophenyl)-N-methyl-1,2,3,4-tetrahydronaphthalen-1-amine. Sertraline may be prepared as described in U.S. Pat. No. 4,536,518, and particularly, in Example 2 of that patent. In embodiments, sertraline is present as a pharmaceutically acceptable salt. For example, in embodiments, sertraline is present as an acid addition salts of various mineral and organic acids such as hydrochloric, hydrobromic, hydroiodide, sulfuric, phosphoric, acetic, lactic, maleic, fumaric, citric, tartaric, succinic, and gluconic and the like. In embodiments, the pharmaceutically acceptable salt of sertraline is hydrochloride salt.

In embodiments, the SSRI is citalopram. Citalopram is (RS)-1-[3-(dimethylamino) propyl]-1-(p-fluorophenyl)-5-phthalancarbonitrile; which was first described in U.S. Pat. No. 4,136,193. This patent publication describes the preparation of citalopram by one method and outlines further methods for preparing citalopram. Citalopram is disclosed to show effects in the treatment of dementia and cerebrovascular disorders, in European patent publication EP 1 169 314 B2. In embodiments, citalopram is present as citalopram hydrobromide.

In embodiments, the SSRI is escitalopram. Escitalopram is the S-enantiomer of citalopram and is a selective, centrally acting serotonin reuptake inhibitor. Its method for preparation is disclosed in U.S. Pat. No. 4,943,590. It shows potent effects in models of neurotic disorders such as anxiolytic effect and prominent effect in the treatment of panic attacks and obsessive-compulsive disorders. In view of the stereo-selectivity, escitalopram is expected to be two times as potent as the racemate in the treatment depression and found to give a faster response than citalopram-racemate in animal models (Willner P., Psychopharmachology 1997, 134, 319-329). Pharmaceutically acceptable acids that can be used for preparing the salt of escitalopram include but not limited to: inorganic acids such as for example hydrochloric acid, hydrobromic acid; and organic acids such as for example acetic acid, nitric acid, sulphuric acid, tartaric acid, oxalic acid, methanesulfonic acid and the like. In embodiments, the pharmaceutically acceptable salt of escitalopram is oxalate salt.

In embodiments, the SSRI is fluoxetine. Fluoxetine, N-methyl-3-(p-trifluoromethylphenoxy)-3-phenylpropylamine, is marketed in the hydrochloride salt form, and as the racemic mixture of its two enantiomers. U.S. Pat. No. 4,314,081 is an early reference on the compound. Robertson et al., J. Med. Chem, 31, 1412 (1988), taught the separation of the R and S enantiomers of fluoxetine and showed that their activity as serotonin uptake inhibitors is similar to each other. In this disclosure, the term “fluoxetine” is used to mean any acid addition salt or the free base, and to include either the racemic mixture or either of the R and S enantiomers.

In embodiments, the SSRI is fluvoxamine. Fluvoxamine, 5-methoxy-1-[4-(trifluoromethyl) phenyl]-1-pentanone O-(2-aminoethyl) oxime, is taught by U.S. Pat. No. 4,085,225. Scientific articles about the drug have been published by Claassen et al., Brit. J. Pharmacol. 60, 505 (1977); and De Wilde et al., J. Affective Disord. 4, 249 (1982); and Benfield et al., Drugs 32, 313 (1986).

In embodiments, the SSRI is paroxetine. Paroxetine, trans-(−)-3-[(1,3-benzodioxol-5-yloxy) methyl]-4-(4-fluorophenyl)piperidine, is found in U.S. Pat. Nos. 3,912,743 and 4,007,196. Reports of the drug's activity are in Lassen, Eur. J. Pharmacol. 47, 351 (1978); Hassan et al., Brit J. Clin. Pharmacol. 19, 705 (1985); Laursen et al., Acta Psychiat. Scand. 71, 249 (1985); and Battegay et al., Neuropsychobiology 13, 31 (1985).

### Selective Norepinephrine Reuptake Inhibitor” or “SNRI”:

Serotonin-noradrenaline reuptake inhibitors (SNRIs) are a class of antidepressant drugs that treat major depressive disorder (MDD), anxiety disorders, obsessive-compulsive disorder (OCD), social phobia, attention-deficit hyperactivity disorder (ADHD), chronic neuropathic pain, fibromyalgia syndrome (FMS), and menopausal symptoms. SNRIs are monoamine reuptake inhibitors; specifically, they inhibit the reuptake of serotonin and noradrenaline, both are neurotransmitters for mood regulation. Like most antidepressants, SNRIs work by ultimately effecting changes in brain chemistry and communication in brain nerve cell circuitry known to regulate mood, to help relieve depression. Exemplary selective norepinephrine reuptake inhibitors (SSRI) include, but are not limited to desvenlafaxine, venlafaxine, levomilnacipran, milnacipran, duloxetine, atomoxetine, sibutramine, reboxetine, tramadol or a pharmaceutically acceptable salt or ester or enantiomer thereof.

In embodiments, the SNRI is desvenlafaxine or venlafaxine. Venlafaxine is known in the literature, and U.S. Pat. No. 4,761,501 teaches its method of synthesis and its activity as an inhibitor of serotonin and norepinephrine uptake. Venlafaxine is identified as compound A in that patent.

In embodiments, the SNRI is milnacipran. Milnacipran (N, N-diethyl-2-aminomethyl-1-phenylcyclopropanecarboxamide) is taught by U.S. Pat. No. 4,478,836, which prepared milnacipran as its Example 4. The patent describes its compounds as antidepressants. Moret et al., Neuropharmacology 24, 1211-19 (1985), describe its pharmacological activities as an inhibitor of serotonin and norepinephrine reuptake.

In embodiments, the SNRI is duloxetine or salt thereof. In embodiments, the SNRI is duloxetine hydrochloride. Duloxetine, N-methyl-3-(1-naphthalenyloxy)-3-(2-thienyl)propanamine, is usually administered as the hydrochloride salt and as the (+) enantiomer. It was first taught by U.S. Pat. No. 4,956,388, which shows its high potency.

The active agents of the present disclosure may be used in the form of the free base or a pharmaceutically acceptable acid addition salt, ester or enantiomer thereof.

### Dosages:

In embodiments, the dosage of dexmedetomidine or a pharmaceutically acceptable salt thereof is in the range of between about 0.5 micrograms to about 300 micrograms. Examples of suitable dosages include: about 0.5 micrograms to about 280 micrograms, about 1 microgram to about 270 micrograms, about 1 microgram to about 260 micrograms, about 1 microgram to about 250 micrograms, about 1 microgram to about 240 micrograms, about 1 microgram to about 230 micrograms, about 1 microgram to about 220 micrograms, about 1 microgram to about 210 micrograms, about 1 microgram to about 200 micrograms, about 1 microgram to about 190 micrograms, about 1 microgram to about 180 micrograms, about 1 microgram to about 170 micrograms, about 1 microgram to about 160 micrograms, about 1 microgram to about 150 micrograms, about 1 microgram to about 140 micrograms, about 1 microgram to about 130 micrograms, about 1 microgram to about 120 micrograms, about 1 microgram to about 110 micrograms, about 1 microgram to about 100 micrograms, about 3 micrograms to about 90 micrograms, about 3 micrograms to about 80 micrograms, about 3 micrograms to 70 micrograms, from about 3 micrograms to about 60 micrograms, from about 3 micrograms to 50 micrograms, from about 3 micrograms to about 40 micrograms, from about 3 micrograms to about 35 micrograms, from about 5 micrograms to about 35 micrograms, about 10 micrograms to about 50 micrograms, about 10 micrograms to about 40 micrograms, about 10 micrograms to about 35 micrograms or about 15 micrograms to 35 micrograms. The dose may be administered one or more times a day including twice, three times, four times, five times or six times per day.

In embodiments, the per unit dose of dexmedetomidine or a pharmaceutically acceptable salt thereof is about 10 micrograms, about 15 micrograms, about 20 micrograms, about 25 micrograms, about 30 micrograms, about 35 micrograms, about 40 micrograms, about 45 micrograms, about 50 micrograms, about 55 micrograms, about 60 micrograms, about 65 micrograms, about 70 micrograms, about 75 micrograms, about 80 micrograms, about 85 micrograms, about 90 micrograms, about 95 micrograms, about 100 micrograms, about 110 micrograms, about 120 micrograms, about 130 micrograms, about 140 micrograms, about 150 micrograms, about 160 micrograms, about 170 micrograms, about 180 micrograms, about 190 micrograms, about 200 micrograms, about 210 micrograms, about 220 micrograms, about 230 micrograms, about 240 micrograms, about 250 micrograms, about 260 micrograms, about 270 micrograms, about 280 micrograms, about 290 micrograms, or about 300 micrograms. In embodiments, dexmedetomidine or a pharmaceutically acceptable salt thereof (e.g. dexmedetomidine hydrochloride) is administered in an amount of about 120 micrograms. In embodiments, dexmedetomidine or a pharmaceutically acceptable salt thereof (e.g. dexmedetomidine hydrochloride) is administered in an amount of about 180 micrograms.

In embodiments, the dosage of dexmedetomidine or a pharmaceutically acceptable salt thereof may be administered twice a day. In embodiments, the dosages of dexmedetomidine or a pharmaceutically acceptable salt thereof are administered at a dose of about 30 micrograms to about 90 micrograms during daytime (e.g., 30 micrograms, 45 micrograms, 60 micrograms, or 90 micrograms) and about 90 micrograms to about 180 micrograms during night-time (e.g., 120 micrograms or 180 micrograms). In embodiments, the dosages of dexmedetomidine or a pharmaceutically acceptable salt thereof are administered at a dose of about 30 micrograms to about 90 micrograms during daytime and 30 micrograms to about 90 micrograms during night-time. In embodiments, the dosages of dexmedetomidine or a pharmaceutically acceptable salt thereof are administered twice a day at a dose of about 90 micrograms to about 180 micrograms during daytime and 30 micrograms to about 90 micrograms during night-time. In embodiments, the dose of dexmedetomidine or a pharmaceutically acceptable salt thereof is about 30 micrograms during day-time and about 90 micrograms during night-time. In embodiments, the dose of dexmedetomidine or a pharmaceutically acceptable salt thereof is about 40 micrograms during day-time and about 80 micrograms during night-time. In embodiments, the dose of dexmedetomidine or a pharmaceutically acceptable salt thereof is about 60 micrograms during day-time and about 120 micrograms during night-time. In embodiments, the dose of dexmedetomidine or a pharmaceutically acceptable salt thereof is about 80 micrograms during day-time and about 180 micrograms during night-time.

In embodiments, dexmedetomidine or a pharmaceutically acceptable salt thereof (e.g. dexmedetomidine hydrochloride) is administered in an amount of about 180 micrograms. In embodiments, dexmedetomidine or a pharmaceutically acceptable salt thereof (e.g. dexmedetomidine hydrochloride) is administered in an amount of about 30 micrograms. In embodiments, dexmedetomidine or a pharmaceutically acceptable salt thereof (e.g. dexmedetomidine hydrochloride) is administered in an amount of about 40 micrograms. In embodiments, dexmedetomidine or a pharmaceutically acceptable salt thereof (e.g. dexmedetomidine hydrochloride) is administered in an amount of about 60 micrograms. In embodiments, dexmedetomidine or a pharmaceutically acceptable salt thereof (e.g. dexmedetomidine hydrochloride) is administered in an amount of about 90 micrograms. In embodiments, dexmedetomidine or a pharmaceutically acceptable salt thereof is administered as 60 micrograms per unit dose twice a day to a total dose of 120 micrograms. For example, a 60 micrograms unit dose is taken in the morning and another 60 micrograms unit dose is taken in the evening or night-time.

The dosage amount of the SSRI/SNRI or other conventional anti-depressants used in the present disclosure depends on the nature of said SSRI/SNRI or other anti-depressants as they differ both in molecular weight and in activity. In embodiment, the SSRI/SNRI is administered at lower doses than normally required. In embodiment, the SSRI/SNRI is administered at normal doses. In embodiments, the dosage of the SSRI/SNRI administered is in the range of between about 1 mg to about 500 mg. Examples of suitable dosages include: about 1 mg to about 300 mg, about 5 mg to about 250 mg, about 5 mg to about 200 mg, about 1 mg to about 190 mg, about 1 mg to about 180 mg, about 1 mg to about 170 mg, about 1 mg to about 160 mg, about 1 mg to about 150 mg, about 1 mg to about 140 mg, about 1 mg to about 130 mg, about 1 mg to about 120 mg, about 1 mg to about 110 mg, about 1 mg to about 100 mg, about 1 mg to about 90 mg, about 1 mg to about 80 mg, about 1 mg to about 70 mg, about 1 mg to about 60 mg, about 1 mg to about 50 mg, about 1 mg to about 40 mg, about 1 mg to about 30 mg, about 1 mg to about 20 mg, about 1 mg to about 10 mg per day. In embodiments, the per unit dose is about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, or about 200 mg of the SSRI/SNRI.

In embodiments, the dosage of sertraline or a pharmaceutically acceptable salt thereof administered is in the range from about 1 mg to about 500 mg per day, although variations may occur depending upon the condition of the subject being treated and the particular route of administration chosen. Examples of suitable dosages of sertraline include: about 10 mg to about 500 mg, about 20 mg to about 500 mg, about 20 mg to about 450 mg, about 25 mg to about 400 mg, about 30 mg to about 350 mg, about 35 mg to about 300 mg, about 40 mg to about 250 mg, about 45 mg to about 200 mg, about 50 mg to about 180 mg, about 55 mg to about 150 mg, about 60 mg to about 100 mg. In embodiments, the daily dose of sertraline or a pharmaceutically acceptable salt thereof is about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 370 mg, about 375 mg, about 380 mg, about 385 mg, about 390 mg, about 395 mg, about 405 mg, about 410 mg, about 415 mg, about 420 mg, about 425 mg, about 430 mg, about 435 mg, about 440 mg, about 445 mg, about 450 mg, about 455 mg, about 460 mg, about 465 mg, about 470 mg, about 475 mg, about 480 mg, about 485 mg, about 490 mg, about 495 mg, or about 500 mg.

In embodiments, the dosage of escitalopram or a pharmaceutically acceptable salt thereof administered is in the range from about 1 mg to about 100 mg per day. Examples of suitable dosages include: about 5 mg to about 100 mg, about 5 mg to about 95 mg, about 10 mg to about 90 mg, about 15 mg to about 85 mg, about 20 mg to about 80 mg, about 25 mg to about 75 mg, about 30 mg to about 70 mg, about 35 mg to about 65 mg, about 40 mg to about 60 mg. In embodiments, the daily dose of escitalopram or a pharmaceutically acceptable salt thereof is about 2 mg, about 5 mg, about 7.5 mg, about 9.5 mg, about 10 mg, about 12 mg, about 15 mg, about 18 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, or about 100 mg. In embodiments, the dosage of escitalopram or a pharmaceutically acceptable salt thereof administered is in the range from about 10 mg to about 20 mg per day.

In embodiments, the dosage of citalopram or a pharmaceutically acceptable salt thereof administered is in the range from about 1 mg to about 100 mg per day. Examples of suitable dosages include: about 5 mg to about 100 mg, about 5 mg to about 95 mg, about 10 mg to about 90 mg, about 15 mg to about 85 mg, about 20 mg to about 80 mg, about 25 mg to about 75 mg, about 30 mg to about 70 mg, about 35 mg to about 65 mg, about 40 mg to about 60 mg. In embodiments, the daily dose of citalopram or a pharmaceutically acceptable salt thereof is about 2 mg, about 5 mg, about 7.5 mg, about 9.5 mg, about 10 mg, about 12 mg, about 15 mg, about 18 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, or about 100 mg. In embodiments, the dosage of citalopram or a pharmaceutically acceptable salt thereof administered is in the range from about 20 mg to about 40 mg per day.

In embodiments, the dosage of fluoxetine or a pharmaceutically acceptable salt thereof administered is in the range from about 1 to about 80 mg, per day. Examples of suitable daily dosages include: about 5 mg to about 80 mg, about 5 mg to about 75 mg, about 10 mg to about 65 mg, about 10 mg to about 60 mg, about 10 mg to about 50 mg, about 10 mg to about 40 mg, about 20 mg to about 60 mg, about 25 mg to about 65 mg, about 30 mg to about 60 mg, about 35 mg to about 65 mg, about 40 mg to about 60 mg. In embodiments, the dosage of fluoxetine or a pharmaceutically acceptable salt thereof administered is in the range from about 10 mg to about 80 mg per day.

In embodiments, the dosage of norfluoxetine or a pharmaceutically acceptable salt thereof administered is in the range from about 0.01 to about 20 mg/kg once per day; preferred, from about 0.05-10 mg/kg once per day, most preferred, from about 0.1-5 mg/kg once per day.

In embodiments, the dosage of paroxetine or a pharmaceutically acceptable salt thereof administered is in the range from about 10 mg to about 80 mg once per day (e.g. 10 mg, 15 mg, 20 mg, 25 mg, 30 mg, 35 mg, 40 mg, 45 mg, 50 mg, 55 mg, 60 mg, 65 mg, 70 mg, 75 mg, 80 mg per day); preferred, from about 20 to about 30 mg once per day. In embodiments, the dosage of fluvoxamine or a pharmaceutically acceptable salt thereof administered is in the range from about 25 mg to about 150 mg (e.g. 25 mg, 50 mg, 75 mg, 100 mg, 125 mg, 150 mg) twice per day.

In embodiments, the dosage of duloxetine or a pharmaceutically acceptable salt thereof administered is in the range from about 10 mg to about 200 mg. Examples of suitable dosages include: about 10 mg to about 180 mg, about 10 mg to about 170 mg, about 10 mg to about 160 mg, about 15 mg to about 200 mg, about 15 mg to about 180 mg, about 15 mg to about 160 mg, about 15 mg to about 150 mg, about 20 mg to about 120 mg, about 20 mg to about 110 mg, about 30 mg to about 110 mg, about 30 mg to about 100 mg, about 30 mg to about 90 mg, about 40 mg to about 80 mg, about 40 mg to about 70 mg, about 50 mg, about 55 mg, about 60 mg, about 65 mg, about 70 mg, about 75 mg, about 80 mg, about 85 mg, about 90 mg, about 95 mg, or about 100 mg. In embodiments, the dosage of duloxetine or a pharmaceutically acceptable salt thereof administered is in the range from about 60 mg to about 120 mg per day.

In embodiments, the SNRI is atomoxetine or a pharmaceutically acceptable salt thereof administered is in the range from about 5 mg/day to about 200 mg/day; about 10 mg to about 190 mg, about 20 mg to about 180 mg, about 30 mg to about 170 mg, about 40 mg to about 160 mg, about 50 mg to about 150 mg preferably in the range from about 60 mg to about 150 mg/day; more preferably from about 60 mg to about 130 mg/day; and still more preferably from about 60 mg to about 120 mg/day.

In embodiments, the dosage of venlafaxine or a pharmaceutically acceptable salt thereof administered is in the range from about 30 mg to about 300 mg per day (for example, 37.5 mg, 75 mg 150 mg). In embodiments, the dosage of venlafaxine or a pharmaceutically acceptable salt thereof administered is in the range from about 75 mg to about 225 mg per day. The dose may be administered twice or thrice a day. In embodiments, the dosage of desvenlafaxine or a pharmaceutically acceptable salt thereof administered is in the range from about 25 mg to about 100 mg per day.

In embodiments, the dosage of levomilnacipran or a pharmaceutically acceptable salt thereof administered is in the range from about 20 mg to about 200 mg per day (For example, 20 mg, 40 mg, 80 mg, 120 mg). In embodiments, the dosage of levomilnacipran or a pharmaceutically acceptable salt thereof administered is in the range from about 20 mg to about 120 mg per day. In embodiments, the dosage of amitriptyline or a pharmaceutically acceptable salt thereof administered is in the range from about 50 mg to about 150 mg per day. In embodiments, the dosage of amitriptyline or a pharmaceutically acceptable salt thereof administered is in the range from about 150 mg to about 300 mg per day. In embodiments, the dosage of doxepin or a pharmaceutically acceptable salt thereof administered is in the range from about 10 mg to about 300 mg per day (for example, 10 mg, 25 mg, 50 mg, 75 mg, 100 mg, 150 mg per day).

In embodiments, the dosage of trimipramine or a pharmaceutically acceptable salt thereof administered is in the range from about 25 mg to about 150 mg per day (for example, 25 mg, 50 mg, 100 mg per day). In embodiments, the dosage of trimipramine a pharmaceutically acceptable salt thereof administered is in the range from about 100 mg to about 200 mg per day. In embodiments, the dosage of imipramine or a pharmaceutically acceptable salt thereof administered is in the range from about 10 mg to about 300 mg per day (for example, 10 mg, 20 mg, 25 mg, 30 mg, 50 mg, 75 mg, 100 mg, 150 mg, 200 mg per day). In embodiments, the dosage of desipramine or a pharmaceutically acceptable salt thereof administered is in the range from about 100 mg to about 300 mg per day. In embodiments, the dosage of desipramine or a pharmaceutically acceptable salt thereof administered is in the range from about 10 mg to about 300 mg per unit (for example, 10 mg, 25 mg, 50 mg, 75 mg, 100 mg, 150 mg).

In embodiments, the dosage of protriptyline or a pharmaceutically acceptable salt thereof administered is in the range from about 5 mg to about 10 mg thrice or four times a day up to a maximum of about 60 mg per day. In embodiments, the dosage of nortriptyline or a pharmaceutically acceptable salt thereof administered is in the range from 50 mg to about 150 mg per day. In embodiments, the dosage of nortriptyline or a pharmaceutically acceptable salt thereof administered is in the range from 10 mg to about 150 mg per unit (for example, about 10 mg, about 25 mg, about 50 mg, about 75 mg).

In embodiments, the dosage of amoxapine or a pharmaceutically acceptable salt thereof administered is in the range from about 200 mg to about 400 mg per day.

In embodiments, the dosage of tranylcypromine or a pharmaceutically acceptable salt thereof administered is in the range from about 10 mg to about 20 mg three times a day. In embodiments, the dosage of tranylcypromine or a pharmaceutically acceptable salt thereof administered is in the range from about 10 mg to about 60 mg per day. In embodiments the dosage of phenelzine or a pharmaceutically acceptable salt thereof administered is in the range from about 30 mg to about 90 mg per day. In embodiments the dosage of phenelzine or a pharmaceutically acceptable salt thereof administered is in the range from about 15 mg to about 90 mg per unit (for example, 15 mg, 30 mg, 45 mg, 60 mg, 75 mg, 90 mg per unit). In embodiments the dosage of isocarboxazide or a pharmaceutically acceptable salt thereof administered is in the range from about 10 mg to about 60 mg per day. In embodiments the dosage of isocarboxazide or a pharmaceutically acceptable salt thereof administered is in the range from about 10 mg to about 60 mg per unit (for example, 10 mg, 20 mg, 30 mg, 40 mg, 50 mg, 60 mg per unit).

In embodiments, the dosage of trazadone or a pharmaceutically acceptable salt thereof administered is in the range from about 50 mg to about 600 mg per day. In embodiments, the dosage of trazadone or a pharmaceutically acceptable salt thereof administered is in the range from about 50 mg to about 600 mg per unit (for example, 50 mg, 100 mg, 150 mg, 200 mg, 250 mg, 300 mg, 350 mg, 400 mg, 450 mg, 500 mg, 550 mg, 600 mg per unit). In embodiments, the dosage of nefazodone or a pharmaceutically acceptable salt thereof administered is in the range from about 150 mg to about 300 mg twice per day. In embodiments, the dosage of nefazodone or a pharmaceutically acceptable salt thereof administered is in the range from about 150 mg to about 300 mg twice per unit (for example. 50 mg, 100 mg, 150 mg, 200 mg, 250 mg, 300 mg per unit).

In embodiments, the dosage of bupropion or a pharmaceutically acceptable salt thereof administered is in the range from about 100 mg to about 450 mg twice or thrice a day. In embodiments, the dosage of bupropion or a pharmaceutically acceptable salt thereof administered is in the range from about 50 mg to about 300 mg per unit (for example, about 50 mg, about 75 mg, about 100 mg, about 200 mg, about 300 mg per unit).

Each unit may be administered to the subject multiple times per day, including once, twice, three times, four times, five times or six times per day. In embodiments, each unit may be administered at an appropriate dosing interval (e.g. about 1 hour between doses) or can be administered concurrently.

The dosages of dexmedetomidine or a pharmaceutically acceptable salt thereof (e.g. hydrochloride salt) and SSRIs or a pharmaceutically acceptable salts, ester or enantiomers thereof (e.g. escitalopram or sertraline and the like), SNRI and other conventional antidepressants to be administered to a particular patient may depend on a variety of factors such as the type and extent of the condition, the overall health status of the particular patient, the particular form of dexmedetomidine and the SSRI/SNRI/other antidepressants being administered, and the particular formulation used to treat the patient.

### Methods and Administration:

In embodiments, there are provided methods of treating major depressive disorder (MDD) in a subject in need thereof, comprising administering to the subject dexmedetomidine or a pharmaceutically acceptable salt thereof as a monotherapy, wherein the subject is non-agitated. In embodiments, the major depressive disorder is associated with anxious distress.

In embodiments, the major depressive disorder is associated with or without a medical condition. In embodiments, the subject has an additional comorbidity or disorder such as obsessive-compulsive disorder, an anxiety disorder, social phobia, PTSD, panic disorder or generalized anxiety disorder. In embodiments, the subject does not have a bipolar disorder. In embodiments, the major depressive disorder is mild or moderate. In embodiments, the major depressive disorder is severe.

In embodiments, the present disclosure provides methods of treating major depressive disorder (MDD) in a non-agitated subject in need thereof, comprising administering oromucosally (includes buccal, sublingual, gingival) about 10 micrograms to about 300 micrograms of dexmedetomidine or a pharmaceutically acceptable salt thereof to the subject.

In embodiments, the present disclosure provides methods of treating major depressive disorder (MDD) in a non-agitated subject in need thereof, comprising administering oromucosally about 20 micrograms to about 200 micrograms of dexmedetomidine or a pharmaceutically acceptable salt thereof to the subject. In embodiments, the present disclosure provides methods of treating major depressive disorder (MDD) in a non-agitated subject in need thereof, comprising administering oromucosally about 30 micrograms to about 180 micrograms of dexmedetomidine or a pharmaceutically acceptable salt thereof to the subject.

In embodiments, the present disclosure provides methods of treating major depressive disorder (MDD) in a non-agitated subject in need thereof, comprising administering oromucosally about 30 micrograms to about 120 micrograms of dexmedetomidine or a pharmaceutically acceptable salt thereof to the subject.

In embodiments, there is provided methods of treating major depressive episodes (MDE) in a subject in need thereof, comprising administering to the subject dexmedetomidine or a pharmaceutically acceptable salt thereof as a monotherapy, wherein the subject is non-agitated. In embodiments, the major depressive episode is associated with anxious distress.

In embodiments, the major depressive episode is associated with or without a medical condition. In embodiments, the subject has an additional comorbidity or disorder such as obsessive-compulsive disorder, an anxiety disorder, social phobia, PTSD, panic disorder or generalized anxiety disorder.

In embodiments, the subject has a bipolar disorder. In embodiments, the subject does not have a bipolar disorder. When the subject has a bipolar disorder the benefits of the methods disclosed herein are particularly valuable. Anti-depressants are contra-indicated for bipolar subjects due to their tendency to induce mania or hypomania in that patient population. This phenomenon is referred to as a “switch” and is associated with poor outcomes.

Advantageously, co-treatment with dexmedetomidine in the induction phase can reduce or eliminate the mania- and hypomania-triggering effect of the anti-depressant. By avoiding the switch of the underlying depression is more effectively treated at least in part because the subjects tend to adhere better to therapeutic regimens. Thus, in embodiments, the disclosure provides methods of reducing switching in subject having a bipolar disorder (e.g., bi-polar I or bi-polar II) by administering dexmedetomidine to the subject. The dexmedetomidine may be administered after anti-depressant therapy is initiated, but preferably the dexmedetomidine and anti-depressant are co-administered. Optionally, the dexmedetomidine may be halted after the induction phase.

In embodiments, the subject is diagnosed with bipolar disorder with anxious distress specifier (DSM-5). In embodiments, the subject is in a manic phase. In embodiments, the subject is in a depressed phase. In embodiments, the subject is in both manic and depressed phases. In embodiments, the major depressive episode is mild or moderate. In embodiments, the major depressive episode is severe.

In embodiments, the present disclosure provides methods of treating major depressive episode (MDE) in a non-agitated subject in need thereof, comprising administering oromucosally (includes buccal, sublingual, gingival) about 10 micrograms to about 300 micrograms of dexmedetomidine or a pharmaceutically acceptable salt thereof to the subject. In embodiments, the present disclosure provides methods of treating major depressive episode (MDE) in a non-agitated subject in need thereof, comprising administering oromucosally about 20 micrograms to about 200 micrograms of dexmedetomidine or a pharmaceutically acceptable salt thereof to the subject. In embodiments, the present disclosure provides methods of treating major depressive episode (MDE) in a non-agitated subject in need thereof, comprising administering oromucosally about 30 micrograms to about 150 micrograms of dexmedetomidine or a pharmaceutically acceptable salt thereof to the subject. In embodiments, the present disclosure provides methods of treating major depressive episode (MDE) in a non-agitated subject in need thereof, comprising administering oromucosally about 30 micrograms to about 120 micrograms of dexmedetomidine or a pharmaceutically acceptable salt thereof to the subject.

In embodiments, dexmedetomidine or a pharmaceutically acceptable salt thereof is administered through routes that include oral, parenteral (e.g. intravenous, subcutaneous, intradermal, intramuscular), topical (includes transdermal), inhalation (e.g. via an aerosol), rectal (e.g., via a suppository), oromucosal, (e.g, buccal, sublingual, gingival), intranasal, vaginal, intrathecal or intraocular.

In embodiments, dexmedetomidine or a salt thereof is administered orally.

In embodiments, dexmedetomidine or a salt thereof is administered through an oral mucosal route (includes, sublingual, buccal or gingival).

In embodiments, dexmedetomidine or pharmaceutically acceptable salt thereof is administered oromucosally as a tablet. In embodiments, the tablet is lyophilized.

In embodiments, dexmedetomidine or pharmaceutically acceptable salt thereof is administered oromucosally as a wafer. In embodiments, dexmedetomidine or pharmaceutically acceptable salt thereof is administered oromucosally as a patch. In embodiments, dexmedetomidine or pharmaceutically acceptable salt thereof is administered oromucosally as a gel or spray or liquid drops. In embodiments, dexmedetomidine or pharmaceutically acceptable salt thereof is administered oromucosally as a film. In embodiments, dexmedetomidine or a pharmaceutically acceptable salt thereof is self-administered by the subject.

In embodiments, dexmedetomidine or a pharmaceutically acceptable salt thereof is administered for an extended period or chronically such as over a period of weeks or months. In embodiments, the dexmedetomidine or a pharmaceutically acceptable salt thereof is administered for a period of at least 7 days, at least 15 days, at least 30 days, at least 60 days, at least 90 days, at least 180 days, at least 365 days, or longer.

In embodiments, the present disclosure provides a method of reducing score on HAM-D scale in a human subject suffering from major depressive disorder, comprising administering oromucosally about 30 micrograms of dexmedetomidine or a pharmaceutically acceptable salt thereof during daytime and about 60 micrograms during night-time, wherein the subject is non-agitated.

In embodiments, the present disclosure provides a method of reducing score on HAM-D scale in a human subject suffering from major depressive disorder, comprising administering oromucosally about 30 micrograms of dexmedetomidine or a pharmaceutically acceptable salt thereof during daytime and about 90 micrograms during night-time, wherein the subject is non-agitated.

In embodiments, the present disclosure provides a method of reducing score on HAM-D scale in a human subject suffering from major depressive disorder, comprising administering oromucosally about 40 micrograms of dexmedetomidine or a pharmaceutically acceptable salt thereof during daytime and about 80 micrograms during night-time, wherein the subject is non-agitated.

In embodiments, the present disclosure provides a method of reducing score on HAM-D scale in a human subject suffering from major depressive disorder, comprising administering oromucosally about 60 micrograms of dexmedetomidine or a pharmaceutically acceptable salt thereof during daytime and about 120 micrograms during night-time, wherein the subject is non-agitated.

In embodiments, the present disclosure provides a method of reducing score on HAM-D scale in a human subject suffering from major depressive disorder, comprising administering oromucosally about 80 micrograms of dexmedetomidine or a pharmaceutically acceptable salt thereof during daytime and about 180 micrograms during night-time, wherein the subject is non-agitated.

In embodiments, the present disclosure provides a method of reducing score on MADRS scale in a human subject suffering from major depressive disorder, comprising administering oromucosally about 30 micrograms of dexmedetomidine or a pharmaceutically acceptable salt thereof during daytime and about 60 micrograms during night-time, wherein the subject is non-agitated.

In embodiments, the present disclosure provides a method of reducing score on MADRS scale in a human subject suffering from major depressive disorder, comprising administering oromucosally about 30 micrograms of dexmedetomidine or a pharmaceutically acceptable salt thereof during daytime and about 90 micrograms during night-time, wherein the subject is non-agitated.

In embodiments, the present disclosure provides a method of reducing score on MADRS scale in a human subject suffering from major depressive disorder, comprising administering oromucosally about 40 micrograms of dexmedetomidine or a pharmaceutically acceptable salt thereof during daytime and about 80 micrograms during night-time, wherein the subject is non-agitated.

In embodiments, the present disclosure provides a method of reducing score on MADRS scale in a human subject suffering from major depressive disorder, comprising administering oromucosally about 60 micrograms of dexmedetomidine or a pharmaceutically acceptable salt thereof during daytime and about 120 micrograms during night-time, wherein the subject is non-agitated.

In embodiments, the present disclosure provides a method of reducing score on HAM-D scale in a human subject suffering from major depressive disorder, comprising administering oromucosally about 80 micrograms of dexmedetomidine or a pharmaceutically acceptable salt thereof during daytime and about 180 micrograms during night-time, wherein the subject is non-agitated.

In embodiments, the present disclosure provides a method of reducing score on HAM-D scale in a human subject suffering from major depressive episode, comprising administering oromucosally about 30 micrograms of dexmedetomidine or a pharmaceutically acceptable salt thereof during daytime and about 60 micrograms during night-time, wherein the subject is non-agitated.

In embodiments, the present disclosure provides a method of reducing score on HAM-D scale in a human subject suffering from major depressive episode, comprising administering oromucosally about 30 micrograms of dexmedetomidine or a pharmaceutically acceptable salt thereof during daytime and about 90 micrograms during night-time, wherein the subject is non-agitated.

In embodiments, the present disclosure provides a method of reducing score on HAM-D scale in a human subject suffering from major depressive episode, comprising administering oromucosally about 40 micrograms of dexmedetomidine or a pharmaceutically acceptable salt thereof during daytime and about 80 micrograms during night-time, wherein the subject is non-agitated.

In embodiments, the present disclosure provides a method of reducing score on HAM-D scale in a human subject suffering from major depressive episode, comprising administering oromucosally about 60 micrograms of dexmedetomidine or a pharmaceutically acceptable salt thereof during daytime and about 120 micrograms during night-time, wherein the subject is non-agitated.

In embodiments, the present disclosure provides a method of reducing score on HAM-D scale in a human subject suffering from major depressive episode, comprising administering oromucosally about 80 micrograms of dexmedetomidine or a pharmaceutically acceptable salt thereof during daytime and about 180 micrograms during night-time, wherein the subject is non-agitated.

In embodiments, the present disclosure provides a method of reducing score on MADRS scale in a human subject suffering from major depressive episode, comprising administering oromucosally about 30 micrograms of dexmedetomidine or a pharmaceutically acceptable salt thereof during daytime and about 60 micrograms during night-time, wherein the subject is non-agitated.

In embodiments, the present disclosure provides a method of reducing score on MADRS scale in a human subject suffering from major depressive episode, comprising administering oromucosally about 30 micrograms of dexmedetomidine or a pharmaceutically acceptable salt thereof during daytime and about 90 micrograms during night-time, wherein the subject is non-agitated.

In embodiments, the present disclosure provides a method of reducing score on MADRS scale in a human subject suffering from major depressive episode, comprising administering oromucosally about 40 micrograms of dexmedetomidine or a pharmaceutically acceptable salt thereof during daytime and about 80 micrograms during night-time, wherein the subject is non-agitated.

In embodiments, the present disclosure provides a method of reducing score on MADRS scale in a human subject suffering from major depressive episode, comprising administering oromucosally about 60 micrograms of dexmedetomidine or a pharmaceutically acceptable salt thereof during daytime and about 120 micrograms during night-time, wherein the subject is non-agitated.

In embodiments, the present disclosure provides a method of reducing score on MADRS scale in a human subject suffering from major depressive episode, comprising administering oromucosally about 80 micrograms of dexmedetomidine or a pharmaceutically acceptable salt thereof during daytime and about 180 micrograms during night-time, wherein the subject is non-agitated.

In embodiments, the present disclosure provides methods for inhibiting serotonin and norepinephrine reuptake and reducing noradrenergic signaling in a human subject, comprising administering a therapeutic amount of dexmedetomidine or a pharmaceutically acceptable salt thereof in combination with a therapeutic amount of a SSRI/SNRI. In embodiments, dexmedetomidine and the SSRI/SNRI are administered in combination from about 1 day to about 28 days. In embodiments, the method comprises (i) an induction phase, comprising administering a therapeutic amount of dexmedetomidine or a pharmaceutically acceptable salt thereof in combination with a therapeutic amount of a SSRI/SNRI from about 1 day to about 28 days, followed by (ii) a maintenance phase, comprising administering to the subject a therapeutic amount of the SSRI/SNRI.

In embodiments, the combination of dexmedetomidine and the SSRI/SNRI produces a synergistic effect such that a lower dose of the SSRIs/SNRIs can be administered to the patient, thereby minimizing the risk of side-effects associated at higher doses of SSRIs/SNRIs.

Certain subjects do not respond to anti-depressants at all or are under-responsive after initial treatment. Thus, in aspects, dexmedetomidine induction may be used with patients who have previously been treated with anti-depressants alone, and found to be non-responsive or under-responsive.

In embodiments, the present disclosure provides a method of treating major depressive disorder in a subject in need thereof, the method comprising: (a) an induction phase, comprising administering to the subject a therapeutic amount of dexmedetomidine or a pharmaceutically acceptable salt thereof in combination with a therapeutic amount of an anti-depressant for at least 1 to 28 days (for example 7 days) followed by (b) a maintenance phase comprising administering to the subject a therapeutic amount of the anti-depressant.

In embodiments, the present disclosure provides a method of treating major depressive episode in a subject in need thereof, the method comprising: (a) an induction phase, comprising administering to the subject a therapeutic amount of dexmedetomidine or a pharmaceutically acceptable salt thereof in combination with a therapeutic amount of an anti-depressant for at least 1 to 28 days (for example 7 days) followed by (b) a maintenance phase comprising administering to the subject a therapeutic amount of the anti-depressant.

In embodiments, the present disclosure provides a method of treating major depressive disorder in a subject in need thereof, the method comprising: (a) an induction phase, comprising administering oromucosally to the subject a therapeutic amount of dexmedetomidine or a pharmaceutically acceptable salt thereof in combination with a therapeutic amount of an anti-depressant administered orally for at least 14 days followed by (b) a maintenance phase comprising administering orally to the subject a therapeutic amount of the anti-depressant.

In embodiments, the present disclosure provides a method of treating major depressive episode in a subject in need thereof, the method comprising: (a) an induction phase, comprising administering oromucosally to the subject a therapeutic amount of dexmedetomidine or a pharmaceutically acceptable salt thereof in combination with a therapeutic amount of an anti-depressant administered orally for at least 14 days followed by (b) a maintenance phase comprising administering orally to the subject a therapeutic amount of the anti-depressant.

In embodiments, the induction phase comprises a treatment period of at least about 1 week. In embodiments, the induction phase comprises a treatment period of about 2 weeks. In embodiments, the induction phase comprises a treatment period of about 3 weeks. In embodiments, the induction phase comprises a treatment period of about 4 weeks or more.

While the induction phase may be halted at 1 week, 2 weeks, 3 weeks or 4 weeks, certain patients may benefit from continued administration of dexmedetomidine. Thus, the induction phase may be 1 month, 2 months, 3 months, 4 months or 6 months.

In embodiments, the anti-depressants include selective serotonin reuptake inhibitor (SSRI), serotonin-norepinephrine reuptake inhibitor (SNRI), tricyclic antidepressants or monoamine oxidase inhibitors. In embodiments, the anti-depressant is SSRI. In embodiments, the anti-depressant is SNRI. In embodiments, the subject has an inadequate or failed response to current antidepressant therapy. In embodiments, the subject is non-agitated. In embodiments, the subject is agitated. In embodiments, the subject also has an anxious distress.

In embodiments, the dexmedetomidine or a pharmaceutically acceptable salt thereof is administered twice daily. In embodiments, the total daily dose of dexmedetomidine or a pharmaceutically acceptable salt thereof is split in a first dose and a second dose. In embodiments, the first dose of dexmedetomidine is administered in the morning and the second dose is administered in the evening. In embodiments, the first dose is lower than the second dose of dexmedetomidine.

In embodiments, the first dose of dexmedetomidine or a pharmaceutically acceptable salt thereof is about 30 micrograms and the second dose of dexmedetomidine is about 60 micrograms. In embodiments, the first dose of dexmedetomidine or a pharmaceutically acceptable salt thereof is about 30 micrograms and the second dose of dexmedetomidine is about 90 micrograms. In embodiments, the first dose of dexmedetomidine or a pharmaceutically acceptable salt thereof is about 40 micrograms and the second dose of dexmedetomidine is about 80 micrograms. In embodiments, the first dose of dexmedetomidine or a pharmaceutically acceptable salt thereof is about 60 micrograms and the second dose of dexmedetomidine is about 120 micrograms. In embodiments, the first dose of dexmedetomidine or a pharmaceutically acceptable salt thereof is about 80 micrograms and the second dose of dexmedetomidine is about 180 micrograms.

In embodiments, the present disclosure provides a method of accelerating an anti-depressant response in a subject with major depressive disorder, comprising administering to the subject a pharmaceutical composition comprising about 30 micrograms to about 200 micrograms of dexmedetomidine or a pharmaceutically acceptable salt thereof and about 5 mg to about 250 mg of an SSRI/SNRI or a pharmaceutically acceptable salt thereof. In embodiments, the subject is non-agitated.

In embodiments, the present disclosure provides a method of accelerating an anti-depressant response in a subject with major depressive disorder, comprising administering to the subject a pharmaceutical composition comprising about 30 micrograms to about 180 micrograms of dexmedetomidine or a pharmaceutically acceptable salt thereof (e.g. dexmedetomidine hydrochloride) and about 25 mg to about 200 mg of sertraline or a pharmaceutically acceptable salt thereof (e.g. sertraline hydrochloride). In embodiments, the subject is non-agitated.

In embodiments, the present disclosure provides a method of accelerating an anti-depressant response in a subject with major depressive disorder, comprising administering to the subject a pharmaceutical composition comprising about 30 micrograms to about 180 micrograms of dexmedetomidine or a pharmaceutically acceptable salt thereof (e.g. dexmedetomidine hydrochloride) and about 5 mg to about 50 mg of escitalopram or a pharmaceutically acceptable salt thereof (escitalopram oxalate). In embodiments, the subject is non-agitated.

In embodiments, the present disclosure provides a method of accelerating an anti-depressant response in a subject with major depressive disorder, comprising administering to the subject a pharmaceutical composition comprising about 30 micrograms to about 180 micrograms of dexmedetomidine or a pharmaceutically acceptable salt thereof (e.g. dexmedetomidine hydrochloride) and about 20 mg to about 160 mg of duloxetine a pharmaceutically acceptable salt thereof. In embodiments, the subject is non-agitated.

In embodiments, the present disclosure provides a method of treating major depressive disorder in a non-agitated subject in need thereof, comprising (a) an induction phase, comprising administering to the subject a therapeutic amount of dexmedetomidine or a pharmaceutically acceptable salt thereof in combination with a therapeutic amount of an SSRI/SNRI from about 1 day to about 28 days, followed by (b) a maintenance phase comprising administering to the subject a therapeutic amount of the SSRI/SNRI.

In embodiments, the present disclosure provides a method of treating major depressive disorder in a non-agitated subject in need thereof, comprising (i) an induction phase, comprising co-administration of an oromucosal dosage form comprising about 30 micrograms to about 180 micrograms of dexmedetomidine or a pharmaceutically acceptable salt thereof and an oral dosage form comprising about 25 mg to about 200 mg of sertraline or a pharmaceutically acceptable salt thereof from about 1 day to about 28 days, followed by (ii) a maintenance phase comprising administering to the subject the oral dosage form comprising about 25 mg to about 200 mg of sertraline or a pharmaceutically acceptable salt thereof.

In embodiments, the present disclosure provides a method of treating major depressive disorder in a non-agitated subject in need thereof, comprising i) an induction phase, comprising co-administration of an oromucosal dosage form comprising about 30 micrograms to about 180 micrograms of dexmedetomidine or a pharmaceutically acceptable salt thereof and an oral dosage form comprising about 10 mg to 80 mg of citalopram or a pharmaceutically acceptable salt thereof from about 1 day to about 28 days, followed by (ii) a maintenance phase comprising administering to the subject the oral dosage form comprising about 10 mg to 80 mg of citalopram or a pharmaceutically acceptable salt thereof.

In embodiments, the present disclosure provides a method of treating major depressive disorder in a non-agitated subject in need thereof, comprising (i) an induction phase, comprising co-administration of an oromucosal dosage form comprising about 30 micrograms to about 180 micrograms of dexmedetomidine or a pharmaceutically acceptable salt thereof and an oral dosage form comprising about 5 mg to about 60 mg of escitalopram or a pharmaceutically acceptable salt thereof from about 1 day to about 28 days, followed by (ii) a maintenance phase comprising administering to the subject the oral dosage form comprising about 5 mg to about 60 mg of escitalopram or a pharmaceutically acceptable salt thereof.

In embodiments, the present disclosure provides a method of treating major depressive disorder in a non-agitated subject in need thereof, comprising (i) an induction phase, comprising co-administration of an oromucosal dosage form comprising about 30 micrograms to about 180 micrograms of dexmedetomidine or a pharmaceutically acceptable salt thereof and an oral dosage form comprising about 20 mg to about 160 mg of duloxetine or a pharmaceutically acceptable salt thereof from about 1 day to about 28 days, followed by (ii) a maintenance phase comprising administering to the subject the oral dosage form comprising about 20 mg to about 120 mg of duloxetine or a pharmaceutically acceptable salt thereof.

In embodiments, the present disclosure provides a method of accelerating an anti-depressant response in a subject with major depressive episode, comprising administering to the subject a pharmaceutical composition comprising about 30 micrograms to about 200 micrograms of dexmedetomidine or a pharmaceutically acceptable salt thereof and about 5 mg to about 250 mg of an SSRI/SNRI or a pharmaceutically acceptable salt thereof. In embodiments, the subject is non-agitated.

In embodiments, the present disclosure provides a method of accelerating an anti-depressant response in a subject with major depressive episode, comprising administering to the subject a pharmaceutical composition comprising about 30 micrograms to about 180 micrograms of dexmedetomidine or a pharmaceutically acceptable salt thereof (e.g. dexmedetomidine hydrochloride) and about 25 mg to about 200 mg of sertraline or a pharmaceutically acceptable salt thereof (e.g. sertraline hydrochloride). In embodiments, the subject is non-agitated.

In embodiments, the present disclosure provides a method of accelerating an anti-depressant response in a subject with major depressive episode, comprising administering to the subject a pharmaceutical composition comprising about 30 micrograms to about 180 micrograms of dexmedetomidine or a pharmaceutically acceptable salt thereof (e.g. dexmedetomidine hydrochloride) and about 5 mg to about 50 mg of escitalopram or a pharmaceutically acceptable salt thereof (escitalopram oxalate). In embodiments, the subject is non-agitated.

In embodiments, the present disclosure provides a method of accelerating an anti-depressant response in a subject with major depressive episode, comprising administering to the subject a pharmaceutical composition comprising about 30 micrograms to about 180 micrograms of dexmedetomidine or a pharmaceutically acceptable salt thereof (e.g. dexmedetomidine hydrochloride) and about 20 mg to about 160 mg of duloxetine a pharmaceutically acceptable salt thereof. In embodiments, the subject is non-agitated.

In embodiments, the present disclosure provides a method of treating major depressive episode in a non-agitated subject in need thereof, comprising (a) an induction phase, comprising administering to the subject a therapeutic amount of dexmedetomidine or a pharmaceutically acceptable salt thereof in combination with a therapeutic amount of an SSRI/SNRI from about 1 day to about 28 days, followed by (b) a maintenance phase comprising administering to the subject a therapeutic amount of the SSRI/SNRI.

In embodiments, the present disclosure provides a method of treating major depressive episode in a non-agitated subject in need thereof, comprising (i) an induction phase, comprising co-administration of an oromucosal dosage form comprising about 30 micrograms to about 180 micrograms of dexmedetomidine or a pharmaceutically acceptable salt thereof and an oral dosage form comprising about 25 mg to about 200 mg of sertraline or a pharmaceutically acceptable salt thereof from about 1 day to about 28 days, followed by (ii) a maintenance phase comprising administering to the subject the oral dosage form comprising about 25 mg to about 200 mg of sertraline or a pharmaceutically acceptable salt thereof.

In embodiments, the present disclosure provides a method of treating major depressive episode in a non-agitated subject in need thereof, comprising i) an induction phase, comprising co-administration of an oromucosal dosage form comprising about 30 micrograms to about 180 micrograms of dexmedetomidine or a pharmaceutically acceptable salt thereof and an oral dosage form comprising about 10 mg to 80 mg of citalopram or a pharmaceutically acceptable salt thereof from about 1 day to about 28 days, followed by (ii) a maintenance phase comprising administering to the subject the oral dosage form comprising about 10 mg to 80 mg of citalopram or a pharmaceutically acceptable salt thereof.

In embodiments, the present disclosure provides a method of treating major depressive episode in a non-agitated subject in need thereof, comprising (i) an induction phase, comprising co-administration of an oromucosal dosage form comprising about 30 micrograms to about 180 micrograms of dexmedetomidine or a pharmaceutically acceptable salt thereof and an oral dosage form comprising about 5 mg to about 60 mg of escitalopram or a pharmaceutically acceptable salt thereof from about 1 day to about 28 days, followed by (ii) a maintenance phase comprising administering to the subject the oral dosage form comprising about 5 mg to about 60 mg of escitalopram or a pharmaceutically acceptable salt thereof.

In embodiments, the present disclosure provides a method of treating major depressive episode in a non-agitated subject in need thereof, comprising (i) an induction phase, comprising co-administration of an oromucosal dosage form comprising about 30 micrograms to about 180 micrograms of dexmedetomidine or a pharmaceutically acceptable salt thereof and an oral dosage form comprising about 20 mg to about 160 mg of duloxetine or a pharmaceutically acceptable salt thereof from about 1 day to about 28 days, followed by (ii) a maintenance phase comprising administering to the subject the oral dosage form comprising about 20 mg to about 120 mg of duloxetine or a pharmaceutically acceptable salt thereof.

In embodiments, the methods described herein reduce at least one sign or symptom of major depressive disorder (MDD). In embodiments, the methods described herein provide reduction in major depressive disorder within 24 hours, 1 week, 1 month, 2 months, 3 months or 12 months after administration.

In embodiments, the methods described herein reduce at least one sign or symptom of major depressive episode (MDE). In embodiments, the methods described herein provide reduction in major depressive episode within 24 hours, 1 week, 1 month, 2 months, 3 months or 12 months after administration.

In embodiments, the sign or symptom is depressed mood, diminished interest in activities, weight loss or gain, decrease or increase in appetite, insomnia or hypersomnia, psychomotor agitation or retardation, fatigue or loss of energy, feelings of worthlessness or excessive or inappropriate guilt, diminished ability to concentrate or indecisiveness, or suicidal ideation or behavior.

In embodiments, the improvement in the subject is measured according to a diary assessment, an assessment by clinician or caregiver, a clinical rating scale, or by functional MRI. In embodiments, the functional MRI measures the amygdala blood oxygen level-dependent (BOLD) response in the subject.

In embodiments, the improvement in the subject is measured using a clinical depression rating scale, wherein the clinical depression rating scale is a Quick Inventory of Depressive Symptomatology (QIDS)-16 scale, a QIDS-16 daily scale, a Hamilton Depression Rating scale (HDRS or HAM-D), a Beck Depression Inventory scale, a Montgomery-Asberg Depression Rating Scale (MADRS), a Clinical Global Impression Scale, a Zung Self-Rating Depression Scale, a Raskin Depression Rating Scale, and/or a Young Mania Rating Scale.

In embodiments, the sign or symptom of major depressive disorder in a subject is measured using the Beck Depression Inventory (BDI). In embodiments, the sign or symptom of major depressive episode in a subject is measured using the Beck Depression Inventory (BDI). The BDI is a 21-item, self-report rating inventory that measures characteristic attitudes and symptoms of depression. The items are rated on 0-3 continuum with 0=no abnormality and 3=severe abnormality. A score of 17-20 indicates borderline clinical depression, a score of 21-30 indicates moderate depression, a score of 31-40 indicates severe depression, and over 40 indicates extreme depression. In embodiments, after treatment with the methods herein, the subject's BDI score decreases by about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment.

In embodiments, the sign or symptom of major depressive disorder in a subject is measured using the Zung Self-Rating Depression Scale. In embodiments, the sign or symptom of major depressive episode in a subject is measured using the Zung Self-Rating Depression Scale. The Zung Self-Rating Depression Scale is a 20-item self-report questionnaire that measures the psychological and somatic symptoms associated with depression. The questionnaire takes about 10 minutes to complete, and items are framed in terms of positive and negative statements. Each item is scored on a Likert scale ranging from 1 to 4. A total score is derived by summing the individual item scores, and ranges from 20 to 80. Most people with depression score between 50 and 69, while a score of 70 and above indicates severe depression. In embodiments, after treatment with the methods herein, the subject's Zung Self-Rating Depression score decreases by about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment.

In embodiments, the sign or symptom of major depressive disorder in a subject is measured using the Raskin Depression Rating Scale. In embodiments, the sign or symptom of major depressive episode in a subject is measured using the Raskin Depression Rating Scale. The Raskin Depression Rating Scale measures baseline levels of depression and change in depression severity overtime. Each item is scored on a scale ranging from 1 to 5 with 1=not at all and 5=very much. A total score is derived by summing the individual item scores, and scores of 9 or greater represents moderate depression. In embodiments, after treatment with the methods herein, the subject's Raskin Depression Rating Scale score decreases by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment.

In embodiments, the sign or symptom of major depressive disorder in a subject is measured using the Inventory of Depressive Symptomatology (IDS). In embodiments, the sign or symptom of major depressive episode in a subject is measured using the Inventory of Depressive Symptomatology (IDS). The IDS is a 30-item inventory that measures depressive signs and symptoms. Each item is scored on a scale of 0 to 3 with 0=absence of pathology and 3=severe pathology. A total score is derived by summing the individual item scores; scores between 26-38 indicates mild depression, scores between 39-48 indicate moderate depression, and scores 49 or greater indicate severe depression. In embodiments, after treatment with the methods described herein, the subject's IDS score decreases by about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment.

In embodiments, the sign or symptom of major depressive disorder in a subject is measured using the Quick Inventory of Depressive Symptomatology (QIDS). In embodiments, the sign or symptom of major depressive episode in a subject is measured using the Quick Inventory of Depressive Symptomatology (QIDS). The QIDS is a 16-item inventory that measures depressive signs and symptoms. Each item is scored on a scale of 0 to 3 with 0=absence of pathology and 3=severe pathology. A total score is derived by summing the individual item scores; scores between 1 1-15 indicate moderate depression, scores between 16-20 indicate severe depression, and scores 21 or greater indicate very severe depression. In embodiments, after treatment with the methods herein, the subject's QIDS score decreases by about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment.

In embodiments, the dexmedetomidine, when administered to a depressed subject, reduces tension, as measured as a PEC item, reduces anxiety, and promotes restorative sleep. Notably, promotion of restorative sleep is not the same as inducing sleep or sedation. Rather, the methods herein provide for relief of sleep inhibition arising from the underlying depression.

In embodiments, the present disclosure provides a method of treatment comprising administering dexmedetomidine or a pharmaceutically acceptable salt to a subject in an oromucosal dosage form that provides rapid relief of anxious distress and then continuing treatment with SSRI/SNRI or a pharmaceutically acceptable salt for an effective period of time.

In embodiments, initial administration of dexmedetomidine along with an SSRI/SNRI for an induction phase of at least about 1 to 28 days treats anxious distress in the patients, which encourages long term administration of the SSRI/SNRI to improve clinical outcomes. In embodiments, the combination of dexmedetomidine and an SSRI/SNRI led to improvement in one or more symptoms of major depressive disorder. and rapid stabilization of anxiety and agitation symptoms compared to treatment with an SSRI/SNRI alone. In embodiments, the combination of dexmedetomidine and an SSRI/SNRI led to improvement in one or more symptoms of major depressive episode compared to treatment with an SSRI/SNRI alone. The administration of dexmedetomidine is effective in treating agitation and anxiety during both the manic and depressed phases. Advantageously, the combination of dexmedetomidine with SSRIs/SNRIs facilitates lowering the doses of SSRIs/SNRIs and therefore reducing the risk of side-effects associated at higher doses of SSRIs/SNRIs.

In embodiments, the present disclosure provides compositions and methods for treating anxious distress (referred to as anxious distress specifier or ADS) in major depressive disorder (MDD). In embodiments, the present disclosure provides compositions and methods for treating anxious distress (referred to as anxious distress specifier or ADS) in major depressive episode (MDE). In embodiments, the present disclosure provides compositions and methods for treating anxious distress in bipolar disorder. In embodiments, the subject is non-agitated. In embodiments, the anxious distress in the subject is measured as per DSM-5 criteria.

The Diagnostic and Statistical Manual of Mental Disorders, Fifth Edition (DSM-5) is the 2013 update to the Diagnostic and Statistical Manual of Mental Disorders, the taxonomic and diagnostic tool published by the American Psychiatric Association (APA). It is the handbook widely used by clinicians and psychiatrists in the United States to diagnose psychiatric illnesses and covers all categories of mental health disorders for both adults and children. The anxious distress specifier (ADS) consists of 5 constructs: 1. Feeling keyed up or tense 2. Feeling unusually restless 3. Difficulty concentrating because of worry 4. Fear that something awful may happen 5. Feeling that the individual might lose control of him/herself. Severity is indicated by the number and types of symptoms: a) Mild: 2 symptoms; b) Moderate: 3 symptoms; c) Moderate-Severe: 4 or 5 symptoms; d) Severe 4 or 5 symptoms with motor agitation. It helps examine the co-occurrence of anxiety and depression symptoms in a single major depressive episode. Major depressive disorder (MDD) patients with anxious features generally have: an earlier age of onset; more persistent course; more severe major depressive episodes (MDEs); increased risk of suicidal ideation & behaviour; poorer quality of life, greater disability; greater personal & socioeconomic costs; and higher rates of treatment failure.

In embodiments, the present disclosure provides a method of treating or preventing anxious distress in a human subject, the method comprising: (a) an induction phase, comprising administering to the subject a therapeutic amount of dexmedetomidine or a pharmaceutically acceptable salt thereof in combination with a therapeutic amount of an SSRI/SNRI from about 1 day to about 28 days, followed by (b) a maintenance phase comprising administering to the subject a therapeutic amount of the SSRI/SNRI.

In embodiments, the induction phase comprising a treatment period of at least about 1 to 4 weeks.

In embodiments, the SSRI/SNRI or a pharmaceutically acceptable salt, ester or enantiomer thereof is administered with starting dose taken as half of the recommended dose for first 2 weeks of the induction phase and the dose is increased to full dose in further weeks depending on tolerability. In embodiments, the anti-depressant effects of treatment by SSRI/SNRI are accelerated by co-administration with dexmedetomidine in accordance with the present disclosure.

In embodiments, the subject has a major depressive disorder (MDD). In embodiments, the subject has a bipolar disorder I. In embodiments, the subject has a bipolar disorder II. In embodiments, the subject has no bipolar disorder. In embodiments, the subject has a previous exposure of SSRI/SNRI. In embodiments, the subject has no previous exposure of SSRI/SNRI. In embodiments, no other treatment is administered to the subject after the administration of dexmedetomidine and SSRI/SNRI. In embodiment, the subject has an additional comorbidity or disorder.

In embodiments, the present disclosure provides a method of treating or preventing anxious distress caused by beginning treatment with a SSRI or SNRI in a human subject, comprising: (a) an induction phase, comprising administering to the subject a therapeutic amount of dexmedetomidine or a pharmaceutically acceptable salt thereof in combination with a therapeutic amount of an SSRI/SNRI from about 1 day to about 28 days, followed by (b) a maintenance phase comprising administering to the subject a therapeutic amount of the SSRI/SNRI.

In embodiments, the present disclosure provides a method of treating or preventing anxious distress caused by beginning treatment with a SSRI or SNRI in a human subject, comprising: (a) an induction phase, comprising oromucosally administering to the subject a therapeutic amount of dexmedetomidine or a pharmaceutically acceptable salt thereof in combination with orally administering a therapeutic amount of an SSRI/SNRI from about 1 day to about 28 days, followed by (b) a maintenance phase comprising orally administering to the subject a therapeutic amount of the SSRI/SNRI.

In embodiments, the anxious distress is significantly reduced within about 1 week of administration. In embodiments, the anxious distress is significantly reduced within about 2 weeks of administration. In embodiments, the SSRI/SNRI in the maintenance phase is administered as a monotherapy.

In embodiments, the SSRI/SNRI in the maintenance phase is administered with an additional SSRI/SNRI. In embodiments, the SSRI/SNRI in the maintenance phase is administered in combination with another therapeutic agent. In embodiments, the SSRI/SNRI in the maintenance phase may be switched to a different SSRI/SNRI after about 2 weeks of administration in case of ineffectiveness or intolerability or to achieve improved outcomes. In embodiments, the maintenance phase is continued until the underlying disease (e.g. major depressive disorder) resolves. In embodiments, the maintenance phase is continued until the underlying disease (e.g. major depressive episode) resolves. In embodiments, the maintenance phase is continued until further treatment is not required (as determined by a clinician or physician). In embodiments, the maintenance phase is continued until the subject experiences a recurrence of anxious distress. In embodiments, the subject is in a manic phase, a depressed phase or both. In embodiments, the subject has undergone previous treatment with SSRIs/SNRIs (e.g., for at least one week) prior to the induction phase. For example, the patient has undergone previous treatment for 1 day, 2 days, 3 days, 4 days, 5 days, 6 days, 7 days, 8 days, 9 days, 10 days, 11 days, 12 days, 13 days, or 14 days prior to the induction phase. In embodiments, the subject has undergone previous treatment with SSRIs/SNRIs for greater than 14 days prior to the induction phase. In embodiments, the subject has undergone previous treatment with SSRIs/SNRIs (e.g., for at least two or three weeks) prior to the induction phase. In embodiments, the subject has undergone previous treatment with SSRIs/SNRIs for greater than 14 days prior to the induction phase. In embodiments, the subject has not undergone any previous treatment with an SSRI/SNRI (i.e., treatment-naive). In embodiments, the subject is not concurrently taking any other anti-depressant treatment therapy. In embodiments, the subject is an adult aged 18 years or above.

In embodiments, the SSRI is selected from the group consisting of sertraline, fluoxetine, norfluoxetine, fluvoxamine, citalopram, escitalopram, dapoxetine, paroxetine, femoxetine, vortioxetine, alaproclate and vilazodone or a pharmaceutically acceptable salt or ester or enantiomer thereof. In embodiments, the SSRI is sertraline. In embodiments, the SSRI is escitalopram.

In embodiments, the SNRI is selected from the group consisting of desvenlafaxine, venlafaxine, levomilnacipran, milnacipran, duloxetine, atomoxetine, sibutramine, reboxetine, tramadol or a pharmaceutically acceptable salt or ester or enantiomer thereof. In embodiments, the SNRI is desvenlafaxine or venlafaxine. In embodiments, the SNRI is duloxetine or salt thereof. In embodiments, the SNRI is duloxetine hydrochloride. In embodiments, the SNRI is atomoxetine.

In embodiments, dexmedetomidine or a pharmaceutically acceptable salt thereof and the SSRI/SNRI are administered in separate unit dosage forms. In embodiments, dexmedetomidine or a pharmaceutically acceptable salt thereof and the SSRI/SNRI are administered sequentially or simultaneously.

In embodiments, the dexmedetomidine and SSRI/SNRI are administered sequentially separated by a time-period via two separate dosage forms. The term specific time-period is meant anywhere within 24 hours of the administration of the other agent, for e.g. 12 hours, 11 hours, 10 hours, 9 hours, 8 hours, 7 hours, 6 hours, 5 hours, 4 hours, 3 hours, 2 hours or 1 hour of each other.

In embodiments, the unit dosage forms are co-administered for about 28 days, about 27 days, about 26 days, about 25 days, about 24 days, about 23 days, about 22 days, about 21 days, about 20 days, about 19 days, about 18 days, about 17 days, about 16 days, about 15 days, about 14 days, about 13 days, about 12 days, about 11 days, about 10 days, about 9 days, about 8 days, about 7 days, about 6 days, about 5 days, about 4 days, about 3 days, about 2 days, or about 1 day. In embodiments, the unit dosage forms are co-administered for about 28 days. In embodiments, the unit dosage forms are co-administered for about 21 days. In embodiments, the unit dosage forms are co-administered for about 14 days. In embodiments, the unit dosage forms are co-administered for about 7 days.

In embodiments, dexmedetomidine or a pharmaceutically acceptable salt thereof is administered at a dose range of about 0.5 micrograms to about 300 micrograms. In embodiments, dexmedetomidine or a pharmaceutically acceptable salt thereof is administered at a dose range of about 0.5 micrograms to about 240 micrograms. In embodiments, dexmedetomidine or a pharmaceutically acceptable salt thereof is administered at a dose of about 120 micrograms. In embodiments, dexmedetomidine or a pharmaceutically acceptable salt thereof is administered at a dose of about 180 micrograms.

In embodiments, the SSRI is administered orally at a dose range of about 5 mg to about 250 mg. In embodiments, the SNRI is administered orally at a dose range of about 10 mg to about 500 mg. In embodiments, the SSRI/SNRI is administered once daily, twice daily, three times a day or more, preferably once, twice or thrice daily for a period of at least 1 week, 2 weeks, 3 weeks, 4 weeks, 5 weeks, 6 weeks or longer till the major depressive disorder resolves.

In embodiments, both unit dosage forms are administered separately by the same or different routes selected from the group consisting of oromucosal (e.g., sublingual or buccal or gingival), oral, topical, transdermal, intranasal, parenteral (e.g., intravenous, intramuscular, subcutaneous) routes and the like.

In embodiments, the present disclosure provides methods of treating or preventing anxious distress in a subject, comprising administering to the subject a pharmaceutical composition comprising about 30 micrograms to about 180 micrograms of dexmedetomidine or a pharmaceutically acceptable salt thereof and about 5 mg to about 250 mg of an SSRI/SNRI or a pharmaceutically acceptable salt thereof. In embodiments, the present disclosure provides methods of treating anxious distress in a subject, comprising administering to the subject a pharmaceutical composition comprising about 30 micrograms to about 180 micrograms of dexmedetomidine or a pharmaceutically acceptable salt thereof and about 10 mg to about 500 mg of an SSRI/SNRI or a pharmaceutically acceptable salt thereof.

In embodiments, the present disclosure provides methods of treating or preventing anxious distress in a subject, comprising administering to the subject a pharmaceutical composition comprising about 30 micrograms to 180 micrograms of dexmedetomidine or a pharmaceutically acceptable salt thereof (e.g. dexmedetomidine hydrochloride) and about 25 mg to about 200 mg of sertraline or a pharmaceutically acceptable salt thereof (e.g. sertraline hydrochloride).

In embodiments, the present disclosure provides methods of treating or preventing anxious distress in a subject, comprising administering to the subject a pharmaceutical composition comprising about 30 micrograms to about 180 micrograms of dexmedetomidine or a pharmaceutically acceptable salt thereof (e.g. dexmedetomidine hydrochloride) and about 5 mg to about 50 mg of escitalopram or a pharmaceutically acceptable salt thereof (e.g. escitalopram oxalate).

In embodiments, the present disclosure provides methods of treating or preventing anxious distress in a subject, comprising administering to the subject a pharmaceutical composition comprising about 30 micrograms to about 180 micrograms of dexmedetomidine or a pharmaceutically acceptable salt thereof (e.g. dexmedetomidine hydrochloride) and about 10 mg to about 500 mg of SNRI.

In embodiments, the present disclosure provides methods of treating or preventing anxious distress in a subject, comprising administering to the subject a pharmaceutical composition comprising about 30 micrograms to about 180 micrograms of dexmedetomidine or a pharmaceutically acceptable salt thereof (e.g. dexmedetomidine hydrochloride) and about 20 mg to about 160 mg of duloxetine or a pharmaceutically acceptable salt thereof.

In embodiments, the anxious distress is significantly reduced within about 3 days. In embodiments, the anxious distress is significantly reduced within about 1 week. In embodiments, the anxious distress is significantly reduced within about 2 weeks.

In embodiments, the present disclosure provides a method of treating or preventing anxious distress in a human subject, comprising (i) an induction phase, comprising co-administration of an oromucosal dosage form comprising about 30 micrograms to about 180 micrograms of dexmedetomidine or a pharmaceutically acceptable salt thereof and an oral dosage form comprising 25 mg to about 200 mg of sertraline or a pharmaceutically acceptable salt thereof from about 1 day to about 28 days, followed by (ii) a maintenance phase comprising administering to the subject the oral dosage form comprising 25 mg to about 200 mg of sertraline or a pharmaceutically acceptable salt thereof. In embodiments, the oromucosal dosage form of dexmedetomidine or a pharmaceutically acceptable salt thereof is a sublingual or buccal or gingival film. In embodiments, the oral dosage form of sertraline or a pharmaceutically acceptable salt thereof is a tablet.

In embodiments, the present disclosure provides a method of treating or preventing anxious distress in a human subject, comprising (i) an induction phase, comprising co-administration of an oromucosal dosage form comprising about 30 micrograms to about 180 micrograms of dexmedetomidine or a pharmaceutically acceptable salt thereof and an oral dosage form comprising about 10 mg to about 80 mg of citalopram or a pharmaceutically acceptable salt thereof from about 1 day to about 28 days, followed by (ii) a maintenance phase comprising administering to the subject the oral dosage form comprising about 10 mg to about 80 mg of citalopram or a pharmaceutically acceptable salt thereof.

In embodiments, the present disclosure provides a method of treating or preventing anxious distress in a human subject, comprising (i) an induction phase, comprising co-administration of an oromucosal dosage form comprising about 30 micrograms to about 180 micrograms of dexmedetomidine or a pharmaceutically acceptable salt thereof and an oral dosage form comprising about 10 mg to about 40 mg citalopram (e.g. 10 mg, 20 mg, 30 mg and 40 mg) or a pharmaceutically acceptable salt thereof from about 1 day to about 28 days, followed by (ii) a maintenance phase comprising administering to the subject the oral dosage form comprising about 5 mg to about 40 mg of citalopram (e.g. 5 mg, 10 mg, 20 mg, 30 mg, 40 mg) or a pharmaceutically acceptable salt thereof. In embodiments, the oromucosal dosage form of dexmedetomidine or a pharmaceutically acceptable salt thereof is a sublingual or buccal or gingival film. In embodiments, the oral dosage form of citalopram or a pharmaceutically acceptable salt thereof is a tablet.

In embodiments, the present disclosure provides a method of treating or preventing anxious distress in a human subject, comprising (i) an induction phase, comprising co-administration of an oromucosal dosage form comprising about 30 micrograms to about 180 micrograms of dexmedetomidine or a pharmaceutically acceptable salt thereof and an oral dosage form comprising about 5 mg to about 60 mg of escitalopram or a pharmaceutically acceptable salt thereof from about 1 day to about 28 days, followed by (ii) a maintenance phase comprising administering to the subject the oral dosage form comprising about 5 mg to about 60 mg of escitalopram or a pharmaceutically acceptable salt thereof.

In embodiments, the present disclosure provides a method of treating or preventing anxious distress in a human subject, comprising (i) an induction phase, comprising co-administration of an oromucosal dosage form comprising about 30 micrograms to about 180 micrograms of dexmedetomidine or a pharmaceutically acceptable salt thereof and an oral dosage form comprising about 10 mg to about 20 mg of escitalopram or a pharmaceutically acceptable salt thereof from about 1 day to about 28 days, followed by (ii) a maintenance phase comprising administering to the subject the oral dosage form comprising about 10 mg to about 20 mg of escitalopram or a pharmaceutically acceptable salt thereof. In embodiments, the oromucosal dosage form of dexmedetomidine or a pharmaceutically acceptable salt thereof is a sublingual or buccal or gingival film. In embodiments, the oral dosage form of escitalopram or a pharmaceutically acceptable salt thereof is a tablet.

In embodiments, the present disclosure provides a method of treating or preventing anxious distress in a human subject, comprising (i) an induction phase, comprising co-administration of an oromucosal dosage form comprising about 30 micrograms to about 180 micrograms of dexmedetomidine or a pharmaceutically acceptable salt thereof and an oral dosage form comprising about 20 mg to about 160 mg of duloxetine or a pharmaceutically acceptable salt thereof from about 1 day to about 28 days, followed by (ii) a maintenance phase comprising administering to the subject the oral dosage form comprising about 20 mg to about 150 mg of duloxetine or a pharmaceutically acceptable salt thereof.

In embodiments, the present disclosure provides a method of treating or preventing anxious distress in a human subject, comprising (i) an induction phase, comprising co-administration of an oromucosal dosage form comprising about 30 micrograms to about 180 micrograms of dexmedetomidine or a pharmaceutically acceptable salt thereof and an oral dosage form comprising about 20 mg to about 160 mg of duloxetine or a pharmaceutically acceptable salt thereof from about 1 day to about 28 days, followed by (ii) a maintenance phase comprising administering to the subject the oral dosage form comprising about 20 mg to about 120 mg of duloxetine or a pharmaceutically acceptable salt thereof. In embodiments, the oromucosal dosage form of dexmedetomidine or a pharmaceutically acceptable salt thereof is a sublingual or buccal or gingival film. In embodiments, the oral dosage form of duloxetine or a pharmaceutically acceptable salt thereof is a tablet. In embodiments, the oral dosage form of duloxetine or a pharmaceutically acceptable salt thereof is a capsule. In embodiments, the oral dosage form of duloxetine or a pharmaceutically acceptable salt thereof is a delayed release capsule.

In embodiments, the methods described herein provide improvement or reduction in anxious distress within 1 week after administration. In embodiments, the methods described herein provide improvement or reduction in anxious distress within 1 month, 2 months, 3 months or 12 months after administration. In embodiments, the anxious distress is associated with major depressive disorder. In embodiments, the anxious distress is associated with major depressive episode.

In embodiments, the methods of the present disclosure provide a reduction in the HAM-A score in a human subject suffering from anxious distress. The HAM-A scale measures the severity of anxiety symptoms and is widely used in both clinical and research settings. The scale consists of 14 items (described below), each defined by a series of symptoms, and measures both psychic anxiety (mental agitation and psychological distress) and somatic anxiety (physical complaints related to anxiety). In embodiments, the subject has a HAM-A total score ≥14 during the start of treatment. In embodiments, the subject experiences a reduction of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, or 56 points as measured by the HAM-A scale. In embodiments, the reduction is experienced in less than one day. In embodiments, the reduction is experienced in 1, 2, 3, 4, 5, 6, 7, 8, 9, 10, 11, 12, 13, 14, 15, 16, 17, 18, 19, 20, 21, 22, 23, 24, 25, 26, 27, or 28 days. In embodiments, the reduction is experienced after 28 days.

1. Anxious mood: Worries, anticipation of the worst, fearful anticipation, irritability.
2. Tension: Feelings of tension, fatigability, startle response, moved to tears easily, trembling, feelings of restlessness, inability to relax.
3. Fears: Of dark, of strangers, of being left alone, of animals, of traffic, of crowds.
4. Insomnia: Difficulty in falling asleep, broken sleep, unsatisfying sleep and fatigue on waking, dreams, nightmares, night terrors.
5. Intellectual: Difficulty in concentration, poor memory.
6. Depressed mood: Loss of interest, lack of pleasure in hobbies, depression, early waking, diurnal swing.
7. Somatic (muscular): Pains and aches, twitching, stiffness, myoclonic jerks, grinding of teeth, unsteady voice, increased muscular tone.
8. Somatic (sensory): Tinnitus, blurring of vision, hot and cold flushes, feelings of weakness, pricking sensation.
9. Cardiovascular symptoms: Tachycardia, palpitations, pain in chest, throbbing of vessels, fainting feelings, missing beat.
10. Respiratory symptoms: Pressure or constriction in chest, choking feelings, sighing, dyspnea.
11. Gastrointestinal symptoms: Difficulty in swallowing, wind abdominal pain, burning sensations, abdominal fullness, nausea, vomiting, borborygmi, looseness of bowels, loss of weight, constipation.
12. Genitourinary symptoms: Frequency of micturition, urgency of micturition, amenorrhea, menorrhagia, development of frigidity, premature ejaculation, loss of libido, impotence.
13. Autonomic symptoms: Dry mouth, flushing, pallor, tendency to sweat, giddiness, tension headache, raising of hair.
14. Behavior at interview: Fidgeting, restlessness or pacing, tremor of hands, furrowed brow, strained face, sighing or rapid respiration, facial pallor, swallowing, etc.

Each item is scored on a 5-point scale, ranging from 0=not present to 4=severe. A rating of 0 indicates that the feeling is not present in the patient. A rating of 1 indicates mild prevalence of the feeling in the patient. A rating of 2 indicates moderate prevalence of the feeling in the patient. A rating of 3 indicates severe prevalence of the feeling in the patient. A rating of 4 indicates a very severe prevalence of the feeling in the patient.

In embodiments, the methods of the present disclosure provide a reduction in the HAM-D score in a human subject suffering from anxious distress. HAM-D (or HDRS) is used as an instrument for assessing the symptoms of depression (Hamilton M. A rating scale for depression. J Neurol Neurosurg Psychiatry 1960 23:56-62). The instrument is administered by clinicians after a structured or unstructured interview of the patient to determine their symptoms. A total score is calculated by summing the individual scores from each question. Scores below 7 generally represent the absence or remission of depression. Scores between 7-17 represent mild depression. Scores between 18-24 represent moderate depression. Scores 25 and above represent severe depression. Most of the studies of depression consider a patient to have experienced ‘response’ to treatment if the score decreases by more than 50%. Remission' is commonly understood to be a score below 7. In embodiments, the subject experiences a reduction of 1, 2, 3, 4, 5, 6, 7, 8, 9, 10, 11, 12, 13, 14, 15, 16, 17, 18, 19, 20, 21, 22, 23, 24, or 25 points as measured by the HAM-D scale. In embodiments, the subject experiences a decrease of greater than 50% in their HAM-D score. In embodiments, the reduction is experienced in less than one day. In embodiments, the reduction is experienced in 1, 2, 3, 4, 5, 6, 7, 8, 9, 10, 11, 12, 13, 14, 15, 16, 17, 18, 19, 20, 21, 22, 23, 24, 25, 26, 27, or 28 days. In embodiments, the reduction is experienced after 28 days. In embodiments, the improvement in symptoms of major depressive disorder is observed as measured by HAM-D-17 depression subscale. In embodiments, the improvement in anxiety and restlessness is observed as measured by HAM-D-17 depression total score and anxiety subscale. In embodiments, the improvement in sleep is assessed with the HAM-D-17 Sleep Subscale. In embodiments, the subject has a HAM-D-17 total score ≥18 during the start of treatment.

In embodiments, the methods of the present disclosure provide a reduction in the Montgomery-Åsberg Depression Rating Scale (MADRS) score in a human subject suffering from anxious distress. The MADRS is a ten-item diagnostic questionnaire which psychiatrists use to measure the severity of major depressive episodes in patients with mood disorders. Higher MADRS score indicates more severe depression, and each item yields a score of 0 to 6. The overall score ranges from 0 to 60. The questionnaire includes questions on the following symptoms 1. Apparent sadness 2. Reported sadness 3. Inner tension 4. Reduced sleep 5. Reduced appetite 6. Concentration difficulties 7. Lassitude 8. Inability to feel 9. Pessimistic thoughts 10. Suicidal thoughts. In embodiments, the subject experiences a reduction of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 points as measured by the MADRS scale. In embodiments, the reduction is experienced in less than one day. In embodiments, the reduction is experienced in 1, 2, 3, 4, 5, 6, 7, 8, 9, 10, 11, 12, 13, 14, 15, 16, 17, 18, 19, 20, 21, 22, 23, 24, 25, 26, 27, or 28 days. In embodiments, the reduction is experienced after 28 days. In embodiments, the subject has a MADRS score ≥20 at the start of treatment (or baseline score).

In embodiments, the methods of the present disclosure provide a reduction in Agitation-Calmness Evaluation Scale (ACES) in a human subject suffering from anxious distress. ACES is a single item measure rating overall agitation and sedation, where 1 indicates marked agitation; 2—moderate agitation; 3—mild agitation; 4—normal behavior; 5—mild calmness; 6—moderate calmness; 7—marked calmness; 8—deep sleep; and 9—unarousable. In embodiments, the present disclosure provides methods of reducing agitation associated with depression to a score of 3 (mild agitation) or 4 (normal behavior), as measured by the Agitation-Calmness Evaluation Scale (ACES). In embodiments, the reduction is experienced in less than one day. In embodiments, the reduction is experienced in 1, 2, 3, 4, 5, 6, 7, 8, 9, 10, 11, 12, 13, 14, 15, 16, 17, 18, 19, 20, 21, 22, 23, 24, 25, 26, 27, or 28 days. In embodiments, the reduction is experienced after 28 days.

In embodiments, the methods of the present disclosure provide a reduction in the Clinical Global Impression (CGI) rating scales. The CGI scales are commonly used measures of symptom severity, treatment response and the efficacy of treatments in treatment studies of patients with mental disorders.

In embodiments, the methods of the present disclosure provide a reduction in CGI-severity scale (CGI-S) in a human subject suffering from anxious distress. CGI-S is a 7-point scale used by the clinician to rate the severity of the patient's illness at the time of assessment, relative to the clinician's past experience with patients who have the same diagnosis. In embodiments, the methods of the present disclosure provide a reduction in CGI-Improvement scale (CGI-I) in a human subject suffering from anxious distress. CGI-I measures the change in agitation in response to treatment. CGI-I scores range from 1 to 7: 0=not assessed (missing), 1=very much improved, 2=much improved, 3=minimally improved, 4=no change, 5=minimally worse, 6=much worse, 7=very much worse. In embodiments, the subject has a clinical Global Impressions-Severity (CGI-S) score of ≥4 during the start of treatment. In embodiments, the subject experiences a reduction of 1, 2, 3, 4, 5, 6, or 7 points as measured by the CGI-S or CGI-I scale. In embodiments, the reduction is experienced in less than one day. In embodiments, the reduction is experienced in 1, 2, 3, 4, 5, 6, 7, 8, 9, 10, 11, 12, 13, 14, 15, 16, 17, 18, 19, 20, 21, 22, 23, 24, 25, 26, 27, or 28 days. In embodiments, the reduction is experienced after 28 days. In embodiments, the CGI-I score is improved to about a 1 (very much improved) or about a 2 (much improved).

### Pharmaceutical Compositions:

In embodiments, the present disclosure provides pharmaceutical compositions, comprising:
(i) about 0.5 micrograms to about 300 micrograms of dexmedetomidine or a pharmaceutically acceptable salt thereof;
(ii) about 5 mg to about 250 mg of an SSRI; and
(iii) one or more pharmaceutically acceptable carriers/excipients.

In embodiments, the present disclosure provides pharmaceutical compositions, comprising:
(i) about 0.5 micrograms to about 300 micrograms of dexmedetomidine or a pharmaceutically acceptable salt thereof;
(ii) about 10 mg to about 500 mg of an SNRI; and
(iii) one or more pharmaceutically acceptable carriers/excipients.

In embodiments, the SSRI is selected from the group consisting of, but not limited to sertraline, fluoxetine, norfluoxetine, fluvoxamine, citalopram, paroxetine, femoxetine, and escitalopram or a pharmaceutically acceptable salt or ester or enantiomer thereof.

In embodiments, the SNRI is selected from the group consisting of desvenlafaxine, venlafaxine, levomilnacipran, milnacipran, duloxetine, atomoxetine, sibutramine, reboxetine, tramadol or a pharmaceutically acceptable salt or ester or enantiomer thereof.

In embodiments, dexmedetomidine or a pharmaceutically acceptable salt thereof is dexmedetomidine hydrochloride.

In embodiments, the composition is formulated as a dosage form selected from the group comprising tablets, orally disintegrating tablets (ODTs), effervescent tablets, capsules, pellets, pills, lozenges or troches, films, powders, dispersible granules, sachets, aqueous solutions, syrups, emulsions, suspensions, solutions, soft gels, dispersions and the like. In embodiments, the composition is formulated as an oromucosal composition. The oromucosal composition of the disclosure may include tablet, capsules, patch or film, sachet, wafers, powders, minitablet, pellet, paste, gel, ointment, cream, drops, liquid (solution, suspension or emulsion), spray, microspheres or nanospheres which can be formulated in accordance with methods that are standard in the art. In embodiments, the oromucosal composition is a film (e.g., a thin film). In embodiments, the film is for sublingual use. In embodiments, the film is for buccal use. In embodiments, the film is for gingival use.

### Films/Patches:

In embodiments, the oromucosal dosage form is in the form of a patch or film (e.g., thin film). The patch or film may have adhesive qualities to prevent movement or swallowing of the patch or film. Suitable film compositions comprising dexmedetomidine are disclosed in U.S. Pat. Nos. 10,792,246, 9,441,142, 9,662,297, 9,585,961, 9,937,123, 9,814,674, 9,248,146, 9,545,376, and 9,662,301; US Patent Appln. Pub. Nos. 2020/0000708, 2020/0172768, and 2021/0077388; and WIPO Patent Appln. Pub. No. 2021/016112A2, the disclosures of each of which are incorporated herein by reference in their entireties.

In embodiments, dexmedetomidine and the SSRI/SNRI or a pharmaceutically acceptable salt thereof are formulated together as a single film product. In embodiments, the film dosage form comprises the SSRI/SNRI and dexmedetomidine or a pharmaceutically acceptable salt thereof either (i) disposed within a polymer matrix or (ii) deposited on the surface of a polymer matrix, e.g., on the surface of a “placebo” film. Further, the SSRI/SNRI and dexmedetomidine or a pharmaceutically acceptable salt thereof may be incorporated as part of a film dosage form in a taste-masked form. In embodiment, particles of drug may be coated or granulated with a taste-masking agent, for example a polymer, oil, or wax. The film dosage forms of the present disclosure may comprise at least one water-soluble polymer that yield films of sufficient film strength (i.e. self-supporting) and rapid disintegration profiles.

The SSRI/SNRI and dexmedetomidine may be present in one or more droplets on the surface of the polymer substrate. The polymer component(s) may, for example, consist of the water-soluble polymer hydroxypropyl cellulose, although different water-soluble polymers are also contemplated. Examples of one or more water-soluble polymers are selected from the group consisting of hydroxypropyl cellulose (HPC), hydroxyethyl cellulose, hydroxypropyl methylcellulose (HPMC), carboxymethyl cellulose, methyl cellulose and mixtures thereof, including mixtures of the same polymer having different molecular weights. Polyethylene oxide (PEO) may also be present herein as a water-soluble polymer in the pharmaceutical film compositions as an example of a pharmaceutically acceptable carrier, or more particularly, as a mucoadhesive agent.

In embodiments, the polymer component consists of a single water-soluble polymer. In some embodiments, the polymer component consists of two or more water-soluble polymers, including two or more of the same water-soluble polymers having different molecular weights.

In embodiments, the water-soluble polymer comprises hydroxypropyl cellulose. In embodiments, the polymer component may consist of one, two or three hydroxypropyl celluloses having different molecular weights. The molecular weights of the different hydroxypropyl celluloses may conveniently range from (i) less than about 60,000 daltons (e.g. about 5,000 daltons to about 49,000 daltons) (ii) about 90,000 daltons to about 200,000 daltons and (iii) about 200,000 daltons to about 500,000 daltons.

In embodiments, the disclosure provides pharmaceutical film compositions comprising: (i) dexmedetomidine or a pharmaceutically acceptable salt thereof, (ii) a polymer component consisting of a first water-soluble polymer having a molecular weight less than about 60,000 daltons (e.g. about 5,000 daltons to about 49,000 daltons), and one or more second-water soluble polymers having a molecular weight greater than about 60,000 daltons; and, optionally, (iii) one or more pharmaceutically acceptable carriers.

In embodiments, the weight ratio of said first water-soluble polymer to said second water-soluble polymer(s) (including PEO when present in the film) in the entire film composition is from about 2:1 to about 1:50, for example about 1:1 to about 1:40, including about 1:1, about 1:2, about 1:3, about 1:4, about 1:5, about 1:6, about 1:7, about 1:8, about 1:9, about 1:10, about 1:11, about 1:12, about 1:13, about 1:14, about 1:15, about 1:16, about 1:17, about 1:18, about 1:19, about 1:20, about 1:21, about 1:22, about 1:23, about 1:24, about 1:25, about 1:26, about 1:27, about 1:28, about 1:29, about 1:30, about 1:31, about 1:32, about 1:33, about 1:34, about 1:35, about 1:36, about 1:37, about 1:38, about 1:39, or about 1:40.

In embodiments, the weight ratio of said first water-soluble polymer to said second water-soluble polymer(s) (including PEO when present in the film) in the entire film composition is from about 1:10 to about 1:30, about 1:15 to about 1:25 or about 1:15 to about 1:20. In some embodiments, a ratio of about 1:15 to about 1:20 provides beneficial functional effects.

Examples of other water-soluble polymers which may be included in the film with the first water-soluble polymer/second water-soluble polymer or replace such polymer(s) include povidone (polyvinylpyrrolidone), copovidone (copolymers of N-vinyl-2-pyrrolidone and vinyl acetate), polyvinyl alcohol, polyethylene glycol, polyacrylic acid, methylmethacrylate copolymer, carboxyvinyl copolymers, polydextrose, pullulan, carboxymethyl cellulose, sodium alginate, chitosan, xanthan gum, tragacanth gum, guar gum, acacia gum, arabic gum, starch, carrageenan, gelatin and mixtures thereof. The water-soluble polymer component, including water-soluble polymer carriers when present, may conveniently comprise about 40% to about 99.8%, about 50% to about 99.7%, about 60% to about 99.6% of the film composition, based on the weight of the film on a dry weight basis.

In embodiments, a polyethylene oxide may be present in the film at about 50% to about 60% w/w of the total film weight.

In embodiments, the present disclosure provides a film composition comprising (e.g. consisting essentially of):
(i) a therapeutically effective amount of dexmedetomidine or a pharmaceutically acceptable salt thereof;
(ii) a polymer component consisting of one or more water-soluble polymers; and
(iii) one or more pharmaceutically acceptable carriers.

The viscosity of deposition solution/suspension may range from about 6 cps to about 3700 cps as measured at 25° C. using a Brookfield viscometer with a small sample adapter. As an example, it may range from about 5 cps to about 500 cps, about 6 cps to about 200 cps, about 6 cps to about 100 cps or about 6 cps to about 50 cps. In one aspect of the present disclosure, the viscosity of the dexmedetomidine composition is from about 6 cps to about 20 cps at 25° C. and a shear rate of about 7 (1/s). The deposition composition may be in any form, including as a solution, emulsion, suspension or dispersion. The oromucosal thin film dosage forms as disclosed herein have several functional advantages to promote rapid onset of drug effect. In embodiments, thin films dosage forms of the disclosure have a disintegration time (DT) of about 15 seconds to about 180 seconds when applied sublingually. A disintegration time in this time-frame provides optimal onset of drug effects.

In embodiments, thin film dosage forms have mucoadhesion properties that provide practical benefits of localizing the film to the sublingual (or buccal or gingival) location and reducing, or preventing, effective removal prior to dissolution.

### Pharmaceutically Acceptable Carriers:

The film compositions may further comprise one or more pharmaceutically acceptable carriers that includes, but is not limited to, liquid carriers, flavours, sweeteners, refreshing agents, antioxidants, pH adjusting agents, permeation enhancers, mucoadhesive agents, plasticizers, bulking agents, surfactants/non-ionic solubilizers, stabilizers, anti-foam agents, colors or the like. In certain embodiments, the film compositions are substantially free of acidic buffer or other acidic agents.

According to one aspect, the pharmaceutically acceptable carrier in the film dosage form includes a liquid carrier. The liquid carrier comprises one or more solvents useful in the preparation of the polymer matrix (drug containing or placebo) and deposition composition in the film composition.

In embodiments, the solvent may be water. In embodiments, the solvent may a polar organic solvent including, but not limited to, ethanol, isopropanol, acetone, butanol, benzyl alcohol and mixtures thereof. In embodiments, the solvent may be a non-polar organic solvent, such as methylene chloride, toluene, ethyl acetate and mixtures thereof. Certain solvents are alcohols, especially ethanol, water and mixtures thereof.

In embodiments, the dosage form is an oromucosal wafer. In embodiments, the wafer is lyophilized. In embodiments, the wafer disintegrates in less than about 1 minute upon contact with an oral mucosa. In embodiments, the wafer disintegrates in not less than about 1 minute upon contact with an oral mucosa. In embodiments, the wafer comprises excipients such as hydroxypropyl cellulose, lactose, mannitol, glycine, and the like.

Carriers suitable for inclusion in other oromucosal formulations include, but are not limited to, sugars, starches, cellulose and its derivatives, malt, gelatin, talc, calcium sulphate, vegetable oils, synthetic oils, polyols, alginic acid, phosphate buffered solutions, emulsifiers, isotonic saline, pyrogen-free water and combinations thereof. Carriers which readily dissolve in saliva may be preferred.

Oromucosal formulations may also include other pharmaceutically acceptable carriers and/or excipients such as binders, lubricants, diluents, coatings, disintegrants, barrier layer components, glidants, colouring agents, solubility enhancers, gelling agents, fillers, proteins, co-factors, emulsifiers, solubilising agents, suspending agents and mixtures thereof. Particular excipients, which may be used according to this disclosure, are known in the art, for example as described in Handbook of Pharmaceutical Excipients, fifth edition, 2005 edited by Rowe et al., Mcgraw Hill.

### Sprays, Drops or Gels:

In embodiments, dexmedetomidine and SSRI/SNRI or a pharmaceutically acceptable salt thereof are formulated as spray compositions or drop compositions suitable for sublingual or buccal or gingival administration and comprise one or more pharmaceutically acceptable liquids (from about 1% to about 99.995% by weight). Such liquids may be solvents, co-solvents, or non-solvents for dexmedetomidine or a pharmaceutically acceptable salt thereof. Examples of pharmaceutically acceptable liquids include water, ethanol, dimethyl sulfoxide, propylene glycol, polyethylene glycol, propylene carbonate, glycerine, N-methylpyrrolidone, pharmaceutically acceptable oils (e.g., soybean, sunflower, peanut, etc.) or the like. In addition to these ingredients, spray or drop formulations may include one or more excipients such as viscosity modulating materials (e.g. polymers, sugars, sugar alcohols, gums, clays, silicas, and the like, such as polyvinylpyrrolidone (PVP)); preservatives (e.g., ethanol, benzyl alcohol, propylparaben and methylparaben); flavoring agents (e.g. peppermint oil), sweeteners (e.g., sugars such as sucrose, glucose, dextrose, maltose, fructose, etc.), artificial sweeteners (e.g. saccharin, aspartame, acesulfame, sucralose), or sugar alcohols (e.g. mannitol, xylitol, lactitol, maltitol syrup); buffers and pH-adjusting agent (e.g., sodium hydroxide, citrate, and citric acid); coloring agents; fragrances, chelating agents (e.g., EDTA); UV absorbers and antifoam agents (e.g., low molecular weight alcohols, dimethicone). In addition to one or more of the aforementioned ingredients suitable for sublingual or buccal sprays or drops, gel formulations may include one or more excipients such as viscosity modulating materials (e.g. water soluble or water swellable polymers such as carbopol, hydroxypropyl cellulose, hydroxypropyl methyl cellulose, carboxymethyl cellulose).

The spray dosage form of the present disclosure for oromucosal administration may include one or more pharmaceutically acceptable liquids (e.g. present in the amount of about 30% to about 99.99% by weight of the composition). Such liquids may be solvents, co-solvents, or non-solvents for dexmedetomidine and SSRI/SNRI or pharmaceutically acceptable salts thereof. Examples of pharmaceutically acceptable liquids include water, ethanol, dimethyl sulfoxide, propylene glycol, polyethylene glycol, propylene carbonate, pharmaceutically acceptable oils (e.g., soybean, sunflower, peanut, peppermint etc.) and the like. The pharmaceutically acceptable liquid is selected either to dissolve the active pharmaceutical ingredient, to produce a stable, homogenous suspension or solution of it, or to form any combination of a suspension or solution.

Sprays, drops, and gels may be made by mixing appropriate quantities of the foregoing ingredients in accordance with standard good manufacturing practices. Such excipients may be included in the formulation to improve patient or subject acceptance or taste, to improve bioavailability, to increase shelf-life, to reduce manufacturing and packaging costs, to comply with requirements of governmental regulatory agencies, and for other purposes. The relative amounts of each ingredient should not interfere with the desirable pharmacological and pharmacokinetic properties of the resulting formulation.

In embodiments, the oromucosal dosage form is in the form of a paste, gel or ointment. The viscosity of the paste, gel or ointment can be adjusted to allow for retention under the tongue or near gums or cheeks or upper lip.

### Tablets/Mini-Tablets:

In embodiments, the present disclosure provides tablet formulations suitable for oromucosal administration (e.g. sublingual or buccal or gingival administration) comprising or consisting essentially of therapeutic amounts of dexmedetomidine, SSRI/SNRI or pharmaceutically acceptable salt thereof and one or more pharmaceutically acceptable carrier (from about 1% to about 99.995% by weight). Such carriers may be taste masking agents, diluents, disintegrants, binders, lubricants, glidants, flavouring agents or liquid solvents.

Examples of pharmaceutically acceptable liquids include water, ethanol, dimethyl sulfoxide, propylene glycol, polyethylene glycol, propylene carbonate, glycerine, N-methylpyrrolidone, pharmaceutically acceptable oils (e.g., soybean, sunflower, peanut, etc.) or the like. Taste masking agents include, for example, amberlite, Opadry® AMB TAN, polymethacrylates (especially Eudragit® L100), sodium starch glycolate (Primojel), carbopol polymers, PEG-5M, sodium acetate, ethylcellulose, betacyclodextrin. Flavouring agents may be, for example, mint powder, menthol, vanillin, aspartame, acesulfame potassium, saccharin. Disintegrants include, for example, sodium starch glycolate, low-substituted hydroxy propyl cellulose, alginic acid, carbon dioxide, carboxymethylcellulose calcium, carboxymethylcellulose sodium, croscarmellose sodium, guar gum, methylcellulose, polacrilin potassium, poloxamer, sodium alginate. Diluents may be, for example, microcrystalline cellulose, dextrates, dextrose, fructose, mannitol, sucralose, sorbitol, starch, pregelatinized starch, sucrose, xylitol, maltose, maltodextrin, maltitol. Binders may be, for example, alginic acid, carbomer, ethyl cellulose, gelatine, liquid glucose, guar gum, hydroxyethyl cellulose, methylcellulose, polydextrose, polyethylene oxide, hydroxypropyl methylcellulose, hydroxypropyl cellulose, sodium alginate.

At least one lubricant may conveniently be incorporated into the formulation to prevent the powder from adhering to tablet punches during the compression procedure. Lubricants may be, for example, talc, magnesium stearate, calcium stearate, glyceryl behenate, hydrogenated castor oil, stearic acid, sodium lauryl sulphate. Glidants are used to promote powder flow by reducing interparticle friction and cohesion. These are used in combination with lubricants as they have no ability to reduce die wall friction. Glidants, may be, for example, colloidal silicon dioxide, calcium silicate, calcium phosphate tribasic.

In embodiments, the oromucosal dosage form is in the form of a tablet or disc or packed powder.

In embodiments, the dosage form is a hard or compressed powdered sublingual or buccal tablet having a low grit component for an organoleptically pleasant mouth feel. The tablet (or particles thereof containing the active agent which can be compressed to form the tablet) may comprise a protective outer coating e.g. any polymer conventionally used in the formation of microparticles and microcapsules.

In embodiments, the dosage form is a sublingual (or buccal or gingival) tablet containing an effervescent agent. Sublingual compositions comprising effervescent agents are disclosed in U.S. Pat. No. 6,200,604, the disclosure of which is incorporated herein by reference in its entireties.

In embodiments, the oromucosal tablet dosage form is prepared by lyophilization (or freeze-drying). A suspension comprising active agent(s) may be prepared with appropriate excipients and the active agent (SSRI/SNRI/dexmedetomidine) suspension may be dispensed into blister packs and freeze-dried. An exemplary freeze-dried preparation platform that could be used for an SSRI/SNRI and/or dexmedetomidine orally disintegrating table (ODT) is the ZYDIS® (Catalent, Somerset, NJ, USA) formulation.

In embodiments, the dosage form is an oromucosal mini-tablet. In embodiments, the mini-tablet disintegrates in less than about 1 minute upon contact with an oral mucosa. In embodiments, the mini-tablet disintegrates in not less than about 1 minute upon contact with an oral mucosa. In embodiments, the minitablet comprises excipients based on co-processed mannitol. In embodiments, the minitablet contains directly compressible excipients.

### Liquids:

In embodiments, the oromucosal dosage form is in a liquid form (e.g. as a solution, suspension or emulsion), and may be, for example, presented as a spray or as drops. In embodiment of the disclosure, SSRI/SNRI and/or dexmedetomidine or pharmaceutically acceptable salts thereof are oromucosally administered in liquid form, e.g. in a flavored or unflavored physiological saline solution. The liquid dosage form may conveniently be administered under the tongue or near the gums or cheeks or upper lip as drops or as a spray.

The solutions include the active ingredient together with a diluent such as water, normal saline, sodium chloride solution, or any other suitable solvent such as propylene glycol, glycerol, ethyl alcohol and so on. The diluent for the solution may particularly be physiological saline solution or water.

The non-solid dosage forms of the disclosure may conveniently be administered by spraying, dripping, painting or squirting the composition under the tongue or near the gums or cheeks or upper lip.

### Intranasal Formulations:

The compositions of the disclosure may be administered to the nasal cavity in any suitable form. For example, the composition may be administered to the nasal cavity in the form of a spray emulsion, suspension or solution, as drops or as a powder.

In embodiment, intranasal compositions of the present disclosure comprise aqueous suspension, solution, or emulsion containing materials in addition to the active ingredient, such as suitable dispersant and/or wetting agent, for example propylene glycol or polyethylene glycol, emulsifier, suspending agent, surfactant, solubilizer, vehicle etc.

The pharmaceutical composition may also be formulated as liposomes, microcapsules or centrosomes, with one or more suitable pharmaceutically acceptable carrier.

Any device that is suitable for intranasal administration can be used. In embodiments, the device is a metered dose device. The metered dose device can deliver a specific dosage amount of the composition. The metered dose device can be a unit-dose, bi-dose, or a multi-dose device. The therapeutic amount that can be administered using a metered dose device can be a unit dose device. The metered dose can be a device that can deliver a pharmaceutical composition intranasally.

### Parenteral Formulations:

Liquid pharmaceutical compositions for parenteral administration may be formulated for administration by injection or continuous infusion. Routes of administration by injection or infusion can include, but are not limited to, intravenous, intraperitoneal, intramuscular, intrathecal, and subcutaneous. In embodiments, parenteral formulations can include prefilled syringes, vials, powder for infusion for reconstitution, concentrate for infusion to be diluted before delivery (ready to dilute) or solutions (ready to use).

Injectable pharmaceutical compositions can be aqueous isotonic solutions or suspensions, and suppositories can be prepared from fatty emulsions or suspensions.

The pharmaceutical compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances.

For intramuscular, intraperitoneal, subcutaneous and intravenous use, sterile solutions of the active ingredient(s) are usually employed, and the pH of the solutions should be suitably adjusted and buffered. For intravenous use, the total concentration of the solute(s) should be controlled to render the preparation isotonic.

The parenteral formulations of the present disclosure can be sterilized. Non-limiting examples of sterilization techniques include filtration through a bacterial-retaining filter, terminal sterilization, incorporation of sterilizing agents, irradiation, and heating.

### Oral Formulations:

The present disclosure includes oral formulations that can be used for delivering dexmedetomidine and/or the SSRI/SNRI or pharmaceutically acceptable salts, ester or enantiomers thereof. Exemplary oral formulations include tablets, orally disintegrating tablets, mouth dissolving tablets, wafers, solution, suspension, emulsions, and capsules.

In embodiment, the present disclosure relates to an oral solid pharmaceutical composition, e.g. in form of a tablet, comprising therapeutic amounts of SSRI/SNRI or dexmedetomidine or both and at least one pharmaceutically acceptable excipient. Tabletting aids, commonly used in tablet formulation can be used and reference is made to the extensive literature on the subject, see in particular Fiedler's “Lexikon der Hilfsstoffe”, 4th Edition, ECV Aulendorf, 1996, which is incorporated herein by reference. These include but are not limited to disintegrants, binders, lubricants, glidants, stabilising agents, fillers or diluents, surfactants and the like.

Disintegrants suitable for compositions of this disclosure include, but are not limited to crosslinked PVP, crospovidone, guar gum, alginic acid, sodium alginate, crosslinked CMC and Ac-Di-Sol®. In embodiments, the disintegrant is crospovidone.

Binders suitable for compositions of this disclosure include, but are not limited to starches, e.g. potato starch, wheat starch, corn starch, celluloses such as microcrystalline cellulose, e.g. products known under the registered trademarks Avicel®, Filtrak®, Heweten® or Pharmacel®, hydroxypropyl cellulose, hydroxyethyl cellulose, and hydroxypropylmethyl cellulose, e.g. hydroxypropyl cellulose having a hydroxypropyl content of 5 to 16% by weight and a molecular weight of from 80, 000 to 1, 150 000, more particularly 140 000 to 850 000.

Glidants suitable for compositions of this disclosure include, but are not limited to colloidal silica, e.g. Aerosil®, magnesium trisilicate, powdered cellulose, starch, talc, and tribasic calcium phosphate.

Fillers or diluents suitable for compositions of this disclosure include, but are not limited to confectioner's sugar, compressible sugar, dextrates, dextrin, dextrose, lactose, mannitol, sorbitol, sucrose, microcrystalline cellulose, in particular having a density of about 0.45 g/cm3, e.g. Avicel®, or powdered cellulose, and talc. In embodiments, the filler is Avicel®.

Lubricants suitable for compositions of this disclosure include, but are not limited to magnesium-, aluminium-, or calcium-stearate, polyethylene glycol (PEG) having a molecular weight of 4,000 to 8,000, and talc.

One or more of these additives may be selected and used by the skilled artisan having regard to the particular desired properties of the solid oral dosage form by routine experimentation and without any undue burden. The amount of each type of additive employed, e.g. glidant, binder, disintegrant, filler or diluent and lubricant may vary within ranges conventional in the art. For example, the amount of glidant may vary within a range of from 0.1 to 10% by weight, in particular 0.1 to 5% by weight, e.g. 0.1 to 0.5% by weight; the amount of binder may vary within a range of from about 10 to 65.3% by weight, e.g. 10 to 45%, e.g. 20 to 30% by weight; the amount of disintegrant may vary within a range of 5 to 60% by weight, e.g. 13 to 50%, e.g. 15 to 40%, e.g. 20 to 30%, e.g. 25%; the amount of filler or diluent may vary within a range of from 15 to 65% by weight e.g. 20 to 50%, e.g. 25 to 40%, e.g. 30%, whereas the amount of lubricant may vary within a range of from 0.1 to 5.0% by weight.

The tablets can be coated by methods well known in the art. The compositions of the invention can be also introduced in microspheres, e.g., fabricated from polyglycolic acid/lactic acid (PGLA).

Liquid preparations for oral administration can take the form of, for example, solutions, syrups, emulsions or suspensions, or they can be presented as a dry product (e.g., powder) for reconstitution with water or other suitable vehicle before use. Preparations for oral administration can be suitably formulated to give controlled or postponed release of the active compound. Controlled or postponed release may apply to one or more of the APIs within the composition and may also apply to portions of one or more of the APIs within the composition.

The APIs can also be administered in the form of liposome delivery systems. Liposomes can be formed from a variety of lipids and phospholipids, such as cholesterol, stearylamine or phosphatidylcholines, as is well known.

### Transdermal Formulations:

The present disclosure includes transdermal formulations that can be used for delivering dexmedetomidine and/or the SSRI/SNRI or pharmaceutically acceptable salts thereof. The formulations can be delivered via the skin for systemic absorption into the bloodstream.

Examples of suitable transdermal forms include, but are not limited to creams, ointments, pastes, gels, and lotions. The creams and ointments may be viscous liquid or semisolid emulsions of either the oil-in-water or water-in-oil type. Pastes comprising absorbable powders dispersed in petroleum or hydrophilic petroleum containing the active ingredient may also be suitable. The pharmaceutical composition may also be formulated as liposomes, microcapsules or centrosomes, with one or more suitable pharmaceutically acceptable carriers.

### Medical Kits:

In embodiments, the present disclosure provides an individual unit dosage form provided as a kit comprising the compositions as described herein in a container with or without instructions for administration to a subject in need thereof. In embodiments, the present disclosure provides a two-unit dosage form provided as a kit comprising the composition as described herein in one or more containers with or without instructions for the simultaneous, sequential or separate administration to a subject in need thereof. In embodiments, the kit comprises a package insert comprising instructions for using the compositions described herein for treatment of anxious distress in a subject.

In embodiments, the kit comprises a package insert comprising instructions for using the compositions described herein for treatment of major depressive disorder in a subject. In embodiments, the kit comprises a package insert comprising instructions for using the compositions described herein for treatment of major depressive episode in a subject. In embodiments, dexmedetomidine or a pharmaceutically acceptable salt thereof and the SSRI/SNRI are each provided in a form that is suitable for administration in conjunction with the other. In embodiments, dexmedetomidine and the SSRI/SNRI or pharmaceutically acceptable salts thereof are provided as a part of the same or single dosage form.

In embodiments, dexmedetomidine and SSRI/SNRI or pharmaceutically acceptable salts thereof are provided as two separate dosage forms for administration of one dosage form prior to, after, and/or at the same time as administration with the other dosage form. When administered sequentially, the sequential administration may be close in time or remote in time.

This may include situations where the two dosage forms are administered (optionally repeatedly) sufficiently closely in time for a beneficial effect for the patient that is greater over the course of the treatment of the relevant condition than if either of the two compositions are administered (optionally repeatedly) alone over the same course of treatment.

When dexmedetomidine and SSRI/SNRI or pharmaceutically acceptable salts thereof are provided as separate dosage forms, each dosage form may be packaged separately for use in conjunction with the other in combination therapy. Alternatively, the two dosage forms may be packaged and presented together as separate components of a “combination pack” for use in conjunction with each other in combination therapy.

In embodiments, the kit comprises a container that includes, but is not limited to bottles, vials (e.g., dual chamber vials), syringes (such as single or dual chamber syringes) and test tubes. The container may be formed from a variety of materials such as glass or plastic. In embodiments, the kit may comprise a label (e.g., on or associated with the container) or a package insert. The label or the package insert may indicate that the compound contained therein may be useful or intended for treating major depressive disorder. The label or the package insert may indicate that the compound contained therein may be useful or intended for treating major depressive episode. The label or the package insert may indicate that the compound contained therein may be useful or intended for treating or preventing anxious distress.

The kit may further comprise other materials desirable from a commercial and user standpoint, including other buffers, diluents, filters, needles, and syringes.

### Specific Embodiments of the Present Disclosure

Embodiment 1. A method of treating major depressive disorder (MDD) in a subject in need thereof, comprising administering to the subject dexmedetomidine or a pharmaceutically acceptable salt thereof as a monotherapy.

Embodiment 2. A method of treating major depressive disorder (MDD) in a non-agitated subject in need thereof, comprising administering oromucosally about 10 micrograms to about 200 micrograms of dexmedetomidine or a pharmaceutically acceptable salt thereof as a monotherapy to the subject.

Embodiment 3. A method of treating major depressive episode (MDE) in a subject in need thereof, comprising administering to the subject dexmedetomidine or a pharmaceutically acceptable salt thereof as a monotherapy.

Embodiment 4. A method of treating major depressive episode (MDE) in a non-agitated subject in need thereof, comprising administering oromucosally about 10 micrograms to about 200 micrograms of dexmedetomidine or a pharmaceutically acceptable salt thereof as a monotherapy to the subject.

Embodiment 5. The method of Embodiment 1 or 2, wherein the major depressive disorder is associated with anxious distress.

Embodiment 6. The method of Embodiment 3 or 4, wherein the major depressive episode is associated with anxious distress.

Embodiment 7. The method of any of Embodiments 1 to 6, wherein the subject is non-agitated at the time of administration of dexmedetomidine.

Embodiment 8. The method of Embodiment 1 or 2, wherein the subject does not have a bipolar disorder.

Embodiment 9. The method of Embodiment 3 or 4, wherein the subject has a bipolar disorder.

Embodiment 10. The method of any of Embodiments 1 to 4, wherein the subject has an additional comorbidity or disorder such as obsessive-compulsive disorder, an anxiety disorder, social phobia, PTSD, panic disorder or generalized anxiety disorder.

Embodiment 11. The method of Embodiment 2 or 4, wherein dexmedetomidine is administered in an amount of about 30 micrograms to about 180 micrograms.

Embodiment 12. The method of Embodiment 2 or 4, wherein dexmedetomidine or a pharmaceutically acceptable salt thereof is administered in an amount of about 10 micrograms, about 20 micrograms, about 30 micrograms, about 40 micrograms, about 60 micrograms, about 80 micrograms, about 90 micrograms, about 100 micrograms, about 120 micrograms, about 180 micrograms or about 200 micrograms.

Embodiment 13. The method of any of Embodiments 2, 4, 11 or 12, wherein dexmedetomidine or a pharmaceutically acceptable salt thereof is administered twice daily in one or more divided doses.

Embodiment 14. The method of any of Embodiments 2, 4, 11 or 12, wherein the total daily dose of dexmedetomidine or a pharmaceutically acceptable salt thereof is split as first dose and second dose.

Embodiment 15. The method of Embodiment 14, wherein the first dose is administered in the morning or daytime and the second dose is administered in the evening or nighttime.

Embodiment 16. The method of Embodiment 14 or 15, wherein the first dose is lower than the second dose of dexmedetomidine.

Embodiment 17. The method of any of Embodiments 14 to 16, wherein the first dose of dexmedetomidine is about 30 micrograms and the second dose is about 90 micrograms.

Embodiment 18. The method of any of Embodiments 14 to 16, wherein the first dose of dexmedetomidine is about 40 micrograms and the second dose is about 80 micrograms.

Embodiment 19. The method of any of Embodiments 1 to 18, wherein dexmedetomidine or a pharmaceutically acceptable salt thereof is administered through routes that include oral, parenteral (e.g. intravenous, subcutaneous, intradermal, intramuscular), topical (includes transdermal administration), inhalation (e.g. via an aerosol), rectal (e.g., via a suppository), oromucosal, intranasal, (buccal, sublingual, gingival), vaginal, intrathecal or intraocular.

Embodiment 20. The method of Embodiment 19, wherein dexmedetomidine or a pharmaceutically acceptable salt thereof is administered oromucosally (includes, sublingual, buccal or gingival).

Embodiment 21. The method of Embodiment 20, wherein dexmedetomidine or a pharmaceutically acceptable salt thereof is administered oromucosally as a tablet.

Embodiment 22. The method of Embodiment, 21 wherein the tablet is lyophilized.

Embodiment 23. The method of Embodiment 20, wherein dexmedetomidine or pharmaceutically acceptable salt thereof is administered oromucosally as a wafer.

Embodiment 24. The method of Embodiment 20, wherein dexmedetomidine or pharmaceutically acceptable salt thereof is administered oromucosally as a patch, gel, liquid drops or a spray.

Embodiment 25. The method of Embodiment 20, wherein dexmedetomidine or pharmaceutically acceptable salt thereof is administered oromucosally as a film.

Embodiment 26. The method of any of preceding embodiments, wherein dexmedetomidine is self-administered.

Embodiment 27. The method of any of preceding Embodiments, wherein dexmedetomidine or a pharmaceutically acceptable salt thereof is administered for extended periods of time.

Embodiment 28. The method of Embodiment 27, wherein dexmedetomidine or a pharmaceutically acceptable salt thereof is administered for a period of at least 7 days, at least 15 days, at least 30 days, at least 60 days, at least 90 days, at least 180 days, at least 365 days, or longer.

Embodiment 29. A method of reducing score on HAM-D scale in a human subject suffering from major depressive disorder, comprising administering oromucosally about 30 micrograms to about 90 micrograms of dexmedetomidine or a pharmaceutically acceptable salt thereof during daytime and about 90 micrograms to about 180 micrograms of dexmedetomidine or a pharmaceutically acceptable salt thereof during night-time, wherein the subject is non-agitated.

Embodiment 30. A method of reducing score on HAM-D scale in a human subject suffering from major depressive episode, comprising administering oromucosally about 30 micrograms to about 90 micrograms of dexmedetomidine or a pharmaceutically acceptable salt thereof during daytime and about 90 micrograms to about 180 micrograms of dexmedetomidine or a pharmaceutically acceptable salt thereof during night-time, wherein the subject is non-agitated.

Embodiment 31. A method of reducing score on MADRS scale in a human subject suffering from major depressive disorder, comprising administering oromucosally about 30 micrograms to about 90 micrograms of dexmedetomidine or a pharmaceutically acceptable salt thereof during daytime and about 90 micrograms to about 180 micrograms of dexmedetomidine or a pharmaceutically acceptable salt thereof during night-time, wherein the subject is non-agitated.

Embodiment 32. A method of reducing score on MADRS scale in a human subject suffering from major depressive episode, comprising administering oromucosally about 30 micrograms to about 90 micrograms of dexmedetomidine or a pharmaceutically acceptable salt thereof during daytime and about 90 micrograms to about 180 micrograms of dexmedetomidine or a pharmaceutically acceptable salt thereof during night-time, wherein the subject is non-agitated.

Embodiment 33. Use of dexmedetomidine or a pharmaceutically acceptable salt thereof as an adjunctive therapy to one or more conventional anti-depressants in subjects with major depressive disorder (MDD).

Embodiment 34. Use of dexmedetomidine or a pharmaceutically acceptable salt thereof as an adjunctive therapy to one or more conventional anti-depressants in subjects with major depressive episode (MDE).

Embodiment 35. Use of dexmedetomidine or a pharmaceutically acceptable salt thereof as an adjunctive therapy to one or more conventional anti-depressants in subjects with anxious distress.

Embodiment 36. A method of accelerating the anti-depressant response in a subject with major depressive disorder (MDD), comprising administering dexmedetomidine or a pharmaceutically acceptable salt thereof in combination with one or more conventional anti-depressants to the subject.

Embodiment 37. A method of accelerating the anti-depressant response in a subject with major depressive episode (MDE), comprising administering dexmedetomidine or a pharmaceutically acceptable salt thereof in combination with one or more conventional anti-depressants to the subject.

Embodiment 38. The method of Embodiment 36 or 37, wherein the subject has failed treatment to conventional anti-depressant therapies.

Embodiment 39. The method of Embodiment 36 or 37, wherein the subject has an inadequate response to conventional anti-depressant therapies.

Embodiment 40. The method of Embodiment 36 or 37, wherein the subject has no previous exposure of any conventional anti-depressant therapy.

Embodiment 41. The method of Embodiment 36 or 37, wherein the subject has an anxious distress.

Embodiment 42. The use or the method of Embodiments 33 to 41, wherein dexmedetomidine or a pharmaceutically acceptable salt thereof is administered immediately the subject is started on conventional antidepressant therapy for the period of about 1 to 4 weeks.

Embodiment 43. The use or the method of embodiments 33 to 41, wherein the conventional antidepressant is selected from the group consisting of but not limited to selective serotonin reuptake inhibitors (SSRI's); selective serotonin and norepinephrine reuptake inhibitors (SNRI's); older tricyclic antidepressants (TCAs); monoamine oxidase inhibitors (MAO-inhibitors), reversible inhibitors of monoamine oxidase (RIMAs), atypical antidepressants; tertiary amine tricyclics and secondary amine tricyclic antidepressants.

Embodiment 44. A method of treating major depressive disorder in a subject in need thereof, the method comprising: (a) an induction phase, comprising administering to the subject a therapeutic amount of dexmedetomidine or a pharmaceutically acceptable salt thereof in combination with a therapeutic amount of an anti-depressant for at least 7 days followed by (b) a maintenance phase comprising administering to the subject a therapeutic amount of the anti-depressant.

Embodiment 45. A method of treating major depressive episode in a subject in need thereof, the method comprising: (a) an induction phase, comprising administering to the subject a therapeutic amount of dexmedetomidine or a pharmaceutically acceptable salt thereof in combination with a therapeutic amount of an anti-depressant for at least 7 days followed by (b) a maintenance phase comprising administering to the subject a therapeutic amount of the anti-depressant.

Embodiment 46. A method of treating major depressive disorder in a subject in need thereof, the method comprising: (a) an induction phase, comprising administering oromucosally to the subject a therapeutic amount of dexmedetomidine or a pharmaceutically acceptable salt thereof in combination with a therapeutic amount of an anti-depressant administered orally for at least 14 days followed by (b) a maintenance phase comprising administering orally to the subject a therapeutic amount of the anti-depressant.

Embodiment 47. A method of treating major depressive episode in a subject in need thereof, the method comprising: (a) an induction phase, comprising administering oromucosally to the subject a therapeutic amount of dexmedetomidine or a pharmaceutically acceptable salt thereof in combination with a therapeutic amount of an anti-depressant administered orally for at least 14 days followed by (b) a maintenance phase comprising administering orally to the subject a therapeutic amount of the anti-depressant.

Embodiment 48. The method of Embodiments 44 to 47, wherein the anti-depressants include selective serotonin reuptake inhibitor (SSRI), serotonin-norepinephrine reuptake inhibitor (SNRI), tricyclic antidepressants or monoamine oxidase inhibitors.

Embodiment 49. The method of any of Embodiments 44 to 48, wherein the subject has an inadequate or failed response to current antidepressant therapy.

Embodiment 50. The method of any of Embodiments 44 to 49, wherein the subject is non-agitated.

Embodiment 51. The method of any of Embodiments 44 to 49, wherein the subject is agitated.

Embodiment 52. The method of any of Embodiments 44 to 51, wherein the subject also has an anxious distress.

Embodiment 53. The method of any of Embodiments 44 to 52, wherein dexmedetomidine or a pharmaceutically acceptable salt thereof is administered twice daily.

Embodiment 54. The method of any of Embodiments 44 to 53, wherein the total daily dose of dexmedetomidine or a pharmaceutically acceptable salt thereof is delivered in a first dose and a second dose.

Embodiment 55. The method of Embodiment 54, wherein the first dose of dexmedetomidine is administered in the morning/daytime and the second dose is administered in the evening/nighttime.

Embodiment 56. The method of Embodiment 54 or 55, wherein the first dose is lower than the second dose of dexmedetomidine.

Embodiment 57. The method of any of Embodiments 54 to 56, wherein the first dose of dexmedetomidine or a pharmaceutically acceptable salt thereof is about 30 micrograms and the second dose of dexmedetomidine is about 60 micrograms.

Embodiment 58. The method of any of Embodiments 54 to 56, wherein the first dose of dexmedetomidine or a pharmaceutically acceptable salt thereof is about 30 micrograms and the second dose is about 90 micrograms.

Embodiment 59. The method of any of Embodiments 54 to 56, wherein the first dose of dexmedetomidine or a pharmaceutically acceptable salt thereof is about 40 micrograms and the second dose is about 80 micrograms.

Embodiment 60. The method of any of Embodiments 54 to 56, wherein the first dose of dexmedetomidine or a pharmaceutically acceptable salt thereof is about 60 micrograms and the second dose is about 120 micrograms.

Embodiment 61. The method of any of Embodiments 54 to 56, wherein the first dose of dexmedetomidine or a pharmaceutically acceptable salt thereof is about 60 micrograms and the second dose is about 180 micrograms.

Embodiment 62. The method of any of Embodiments 44 to 47, wherein the induction phase comprising a treatment period of at least about 1 or 2 weeks.

Embodiment 63. The method of any of Embodiments 44 to 47, wherein the induction phase comprising a treatment period of at least about 3 weeks.

Embodiment 64. The method of any of Embodiments 44 to 47, wherein the induction phase comprising a treatment period of at least about 4 weeks.

Embodiment 65. The method of any of Embodiments 44, 46, 48 to 64, reduce at least one sign or symptom of major depressive disorder.

Embodiment 66. The method of any of Embodiments 45 and 47 to 64, reduce at least one sign or symptom of major depressive episode.

Embodiment 67. The method of Embodiment 65, wherein the method provide reduction in major depressive disorder within 24 hours, 1 week, 1 month, 2 months, 3 months or 12 months after administration.

Embodiment 68. The method of Embodiment 66, wherein the method provide reduction in major depressive episode within 24 hours, 1 week, 1 month, 2 months, 3 months or 12 months after administration.

Embodiment 69. The method of Embodiment 65 or 66, wherein the sign or symptom is depressed mood, diminished interest in activities, weight loss or gain, decrease or increase in appetite, insomnia or hypersomnia, psychomotor agitation or retardation, fatigue or loss of energy, feelings of worthlessness or excessive or inappropriate guilt, diminished ability to concentrate or indecisiveness, or suicidal ideation or behavior.

Embodiment 70. The method of any of Embodiments 44 to 69, wherein the improvement in the subject is measured according to a diary assessment, an assessment by clinician or caregiver, a clinical rating scale, or by functional MRI.

Embodiment 71. The method of any of Embodiments 44 to 69, wherein the improvement in the subject is measured using a clinical depression rating scale, wherein the clinical depression rating scale is a Quick Inventory of Depressive Symptomatology (QIDS)-16 scale, a QIDS-16 daily scale, a Hamilton Depression Rating scale (HDRS or HAM-D), a Beck Depression Inventory scale, a Montgomery-Asberg Depression Rating Scale (MADRS), a Clinical Global Impression Scale, a Zung Self-Rating Depression Scale, a Raskin Depression Rating Scale, and/or a Young Mania Rating Scale.

Embodiment 72. A method of reducing score on HAM-D scale in a human subject suffering from major depressive disorder, comprising administering oromucosally an effective amount of dexmedetomidine or a pharmaceutically acceptable salt thereof in combination with an orally effective amount of SSRI/SNRI.

Embodiment 73. A method of reducing score on HAM-D scale in a human subject suffering from major depressive episode, comprising administering oromucosally an effective amount of dexmedetomidine or a pharmaceutically acceptable salt thereof in combination with an orally effective amount of SSRI/SNRI.

Embodiment 74. The method of Embodiment 72 or 73, wherein the subject has a HAM-D-17 total score ≥18 at the start of treatment (or baseline).

Embodiment 75. The method of Embodiment 72 or 73, wherein subject has a total Score of ≥14 on the Hamilton Anxiety Scale (HAM-A) at the start of treatment (or baseline).

Embodiment 76. The method of embodiment 72 or 73, providing a reduction in HAM-D score of about 5% and about 100%, for example, about 5%, about 10%, about 15%, about 20%, about 25%, about 30%, about 35%, about 40%, about 45%, about 50%, about 55% or more, compared to baseline score observed prior to treatment.

Embodiment 77. A method of reducing score on MADRS scale in a human subject suffering from major depressive disorder, comprising administering oromucosally effective amount of dexmedetomidine or a pharmaceutically acceptable salt thereof in combination with an orally effective amount of SSRI/SNRI.

Embodiment 78. A method of reducing score on MADRS scale in a human subject suffering from major depressive episode, comprising administering oromucosally effective amount of dexmedetomidine or a pharmaceutically acceptable salt thereof in combination with an orally effective amount of SSRI/SNRI.

Embodiment 79. The method of Embodiment 77 or 78, providing a reduction in MADRS score of about 5% and about 100%, for example, about 5%, about 10%, about 15%, about 20%, about 25%, about 30%, about 35%, about 40%, about 45%, about 50%, or more, compared to baseline score of about ≥20 as observed prior to treatment.

Embodiment 80. The method of any of Embodiments 72 to 79, providing a relief of sleep inhibition arising from the underlying disorder.

Embodiment 81. A method of treatment comprising administering dexmedetomidine or a pharmaceutically acceptable salt to a subject in an oromucosal dosage form that provides rapid relief of anxious distress and then continuing treatment with SSRI/SNRI or a pharmaceutically acceptable salt for an effective period of time.

Embodiment 82. A method of treating or preventing anxious distress in a human subject, comprising:
(i) an induction phase, comprising administering to the subject a therapeutic amount of dexmedetomidine or a pharmaceutically acceptable salt thereof in combination with a therapeutic amount of a SSRI/SNRI from about 1 day to about 28 days, followed by
(ii) a maintenance phase comprising administering to the subject a therapeutic amount of the SSRI/SNRI.

Embodiment 83. The method of Embodiment 82, wherein the subject has a major depressive disorder.

Embodiment 84. The method of Embodiment 82, wherein the subject has a bipolar disorder.

Embodiment 85. The method of Embodiment 82, wherein the subject is non-agitated.

Embodiment 86. The method of Embodiment 82, wherein the subject is agitated.

Embodiment 87. The method of Embodiment 82, wherein the induction phase comprising a treatment period of at least about 1 to 4 weeks.

Embodiment 88. The method of Embodiment 82, wherein the subject had a previous exposure of SSRI/SNRI.

Embodiment 89. The method of Embodiment 82, wherein the subject had no previous exposure of SSRI/SNRI.

Embodiment 90. The method of Embodiment 82, wherein no other treatment is administered to the subject after administration of dexmedetomidine and SSRI/SNRI.

Embodiment 91. The method of Embodiment 82, wherein the subject has an additional comorbidity or disorder.

Embodiment 92. A method of treating or preventing anxious distress caused by beginning treatment with a SSRI/SNRI in a human subject, the method comprising:
(a) an induction phase, comprising administering to the subject a therapeutic amount of dexmedetomidine or a pharmaceutically acceptable salt thereof in combination with a therapeutic amount of a SSRI/SNRI from about 1 day to about 28 days, followed by
(b) a maintenance phase comprising administering to the subject a therapeutic amount of the SSRI/SNRI.

Embodiment 93. A method of treating or preventing anxious distress caused by beginning treatment with a SSRI or SNRI in a human subject, comprising: (a) an induction phase, comprising oromucosally administering to the subject a therapeutic amount of dexmedetomidine or a pharmaceutically acceptable salt thereof in combination with an oral administration of therapeutic amount of an SSRI/SNRI from about 1 day to about 28 days, followed by (b) a maintenance phase comprising orally administering to the subject a therapeutic amount of the SSRI/SNRI.

Embodiment 94. The method of Embodiment 92 or 93, wherein the anxious distress is significantly reduced within about 1 week of administration.

Embodiment 95. The method of Embodiment 92 or 93, wherein the anxious distress is significantly reduced within about 2 weeks of administration.

Embodiment 96. The method of Embodiment 92 or 93, wherein the SSRI/SNRI in the maintenance phase is administered as a monotherapy.

Embodiment 97. The method of Embodiment 92 or 93, wherein the SSRI/SNRI in the maintenance phase is administered with an additional SSRI/SNRI.

Embodiment 98. The method of Embodiment 92 or 93, wherein the maintenance phase is continued until the underlying disease (e.g. major depressive disorder) resolves.

Embodiment 99. The method of Embodiment 92 or 93, wherein the maintenance phase is continued until the subject experiences a recurrence of anxious distress.

Embodiment 100. The method of Embodiment 92 or 93, wherein the subject is in a manic phase, a depressed phase or both.

Embodiment 101. The method of Embodiment 92 or 93, wherein the subject has undergone previous treatment with SSRIs/SNRIs (e.g., for at least one week) prior to the induction phase.

Embodiment 102. The method according to Embodiment 92 or 93, wherein the SSRI is selected from the group consisting of sertraline, fluoxetine, norfluoxetine, fluvoxamine, citalopram, escitalopram, dapoxetine, paroxetine, femoxetine, vortioxetine, alaproclate and vilazodone or a pharmaceutically acceptable salt or ester or enantiomer thereof.

Embodiment 103. The method according to Embodiment 102, wherein the SSRI is sertraline or a pharmaceutically acceptable salt thereof.

Embodiment 104. The method according to Embodiment 102, wherein the SSRI is escitalopram or a pharmaceutically acceptable salt thereof.

Embodiment 105. The method according to Embodiment 102, wherein the SSRI is citalopram.

Embodiment 106. The method according to Embodiment 92 or 93, wherein the SNRI is selected from the group consisting of desvenlafaxine, venlafaxine, levomilnacipran, milnacipran, duloxetine, atomoxetine, sibutramine, reboxetine, tramadol or a pharmaceutically acceptable salt or ester or enantiomer thereof.

Embodiment 107. The method according to Embodiment 106, wherein the SNRI is desvenlafaxine or venlafaxine or a pharmaceutically acceptable salt thereof.

Embodiment 108. The method according to Embodiment 107, wherein the SNRI is duloxetine or a pharmaceutically acceptable salt thereof (e.g. duloxetine hydrochloride).

Embodiment 109. The method according to Embodiment 107, wherein the SNRI is atomoxetine or a pharmaceutically acceptable salt thereof.

Embodiment 110. A method of treating or preventing anxious distress in a human subject, comprising (i) an induction phase, comprising co-administration of an oromucosal dosage form comprising about 30 micrograms to about 180 micrograms of dexmedetomidine or a pharmaceutically acceptable salt thereof and an oral dosage form comprising 25 mg to about 200 mg of sertraline or a pharmaceutically acceptable salt thereof from about 1 day to about 28 days, followed by (ii) a maintenance phase comprising administering to the subject the oral dosage form comprising a 25 mg to about 200 mg of sertraline or a pharmaceutically acceptable salt thereof.

Embodiment 111. A method of treating or preventing anxious distress in a human subject, comprising (i) an induction phase, comprising co-administration of an oromucosal dosage form comprising about 30 micrograms to about 180 micrograms of dexmedetomidine or a pharmaceutically acceptable salt thereof and an oral dosage form comprising about 20 mg to about 40 mg of citalopram or a pharmaceutically acceptable salt thereof from about 1 day to about 28 days, followed by (ii) a maintenance phase comprising administering to the subject the oral dosage form comprising about 20 mg to about 40 mg of citalopram or a pharmaceutically acceptable salt thereof.

Embodiment 112. A method of treating or preventing anxious distress in a human subject, comprising (i) an induction phase, comprising co-administration of an oromucosal dosage form comprising about 30 micrograms to about 180 micrograms of dexmedetomidine or a pharmaceutically acceptable salt thereof and an oral dosage form comprising about 10 mg to about 20 mg of escitalopram or a pharmaceutically acceptable salt thereof from about 1 day to about 28 days, followed by (ii) a maintenance phase comprising administering to the subject the oral dosage form comprising about 10 mg to about 20 mg of escitalopram or a pharmaceutically acceptable salt thereof.

Embodiment 113. The method of Embodiment 111 or 112, wherein the oral dosage form of citalopram or escitalopram or a pharmaceutically acceptable salt thereof is a tablet.

Embodiment 114. A method of treating or preventing anxious distress in a human subject, comprising (i) an induction phase, comprising co-administration of an oromucosal dosage form comprising about 30 micrograms to about 180 micrograms of dexmedetomidine or a pharmaceutically acceptable salt thereof and an oral dosage form comprising about 20 mg to about 160 mg of duloxetine or a pharmaceutically acceptable salt thereof from about 1 day to about 28 days, followed by (ii) a maintenance phase comprising administering to the subject the oral dosage form comprising about 20 mg to about 120 mg of duloxetine or a pharmaceutically acceptable salt thereof.

Embodiment 115. The method of Embodiment 114, wherein the oral dosage form of duloxetine is a tablet or a capsule (e.g. delayed release capsule).

Embodiment 116. The method of Embodiments 110 to 115, wherein the oromucosal dosage form of dexmedetomidine or a pharmaceutically acceptable salt thereof is a sublingual or buccal or gingival film.

Embodiment 117. The method according to any of embodiments 33 to 116, wherein dexmedetomidine or a pharmaceutically acceptable salt thereof and the SSRI/SNRI are administered in separate unit dosage forms.

Embodiment 118. The method according to Embodiment 117, wherein both unit dosage forms are administered to the subject separately by the same or different routes selected from the group consisting of oromucosal (e.g., sublingual or buccal or gingival), oral, topical, transdermal, intranasal, parenteral (e.g., intravenous, intramuscular, subcutaneous) routes and the like.

Embodiment 119. The method according to Embodiment 118, wherein both unit dosage forms are administered through the oromucosal route.

Embodiment 120. The method according to Embodiment 117, wherein the SSRI/SNRI is administered in a dosage form selected from the group comprising tablets, orally disintegrating tablets (ODTs), effervescent tablets, capsules, pellets, pills, lozenges or troches, films, powders, dispersible granules, catchets, aqueous solutions, syrups, emulsions, suspensions, solutions, soft gels, dispersions and the like.

Embodiment 121. The method according to Embodiment 120, wherein the SSRI/SNRI is administered orally.

Embodiment 122. The method according to Embodiment 120 or 121, wherein the SSRI/SNRI is administered as an oral tablet.

Embodiment 123. The method according to Embodiment 117, wherein the dexmedetomidine or a pharmaceutically acceptable salt thereof (e.g. dexmedetomidine hydrochloride) is administered oromucosally in a dosage form selected from tablet, film, spray, gel or drops, preferably film.

Embodiment 124. The method according to any of Embodiments 117 to 123, wherein said dosage forms are co-administered for about 28 days.

Embodiment 125. The method according to any of Embodiments 117 to 123, wherein said dosage forms are co-administered for about 21 days.

Embodiment 127. The method according to any of Embodiments 117 to 123, wherein said dosage forms are co-administered for about 14 days.

Embodiment 128. The method according to any of Embodiments 117 to 123, said dosage forms are co-administered for about 7 days.

Embodiment 129. The method according to Embodiments 33 to 116, wherein dexmedetomidine or a pharmaceutically acceptable salt thereof (e.g. dexmedetomidine hydrochloride) and the SSRI/SNRI are administered together in a single unit dosage form.

Embodiment 130. The method according to embodiment 129, wherein said unit dosage form is administered to the subject through the oromucosal route in the form of tablet, film, spray, gel or drops.

Embodiment 131. The method according to Embodiment 130, wherein the dosage form is a film and the film is a thin film.

Embodiment 132. The method according to any of Embodiments 129 to 131, wherein said dosage form is administered for about 28 days.

Embodiment 133. The method according to any of Embodiments 129 to 131, wherein said dosage form is administered for about 21 days.

Embodiment 134. The method according to any of Embodiments 129 to 131, wherein said dosage form is administered for about 14 days.

Embodiment 135. The method according to any of Embodiments 129 to 131, wherein said dosage form is administered for about 7 days.

Embodiment 136. The method according to any of Embodiments 33 to 135, wherein said dosage form is administered once a day or multiple times a day.

Embodiment 137. The method according to any one of Embodiments 1 to 136, wherein dexmedetomidine a pharmaceutically acceptable salt thereof is administered in a dosage amount in a range of about 0.5 micrograms to about 300 micrograms (e.g. 10 micrograms to 240 micrograms).

Embodiment 138. The method according to Embodiment 137, wherein dexmedetomidine or a pharmaceutically acceptable salt thereof is administered as a single dose containing about 180 micrograms dexmedetomidine or a pharmaceutically acceptable salt thereof.

Embodiment 139. The method according to Embodiment 137, wherein dexmedetomidine or a pharmaceutically acceptable salt thereof is administered as a single dose containing about 120 micrograms dexmedetomidine or a pharmaceutically acceptable salt thereof.

Embodiment 140. The method according to any of Embodiments 1 to 139, wherein dexmedetomidine or a pharmaceutically acceptable salt thereof is dexmedetomidine hydrochloride.

Embodiment 141. The method according to any of Embodiments 1 to 136, wherein the SSRI is administered in a dosage amount in a range of about 5 mg to about 250 mg.

Embodiment 142. The method according to any of Embodiments 1 to 136, wherein the SNRI is administered in a dosage amount in a range of about 10 mg to about 500 mg.

Embodiment 143. The method according to any of Embodiments 44 to 114, wherein the maintenance phase is continued until the underlying disease (e.g. major depressive disorder) resolves or until the subject experiences the recurrence of anxious distress.

Embodiment 144. The method according to any of Embodiments 44 to 143, wherein the subject is in a manic phase, a depressed phase or both.

Embodiment 145. The method according to any of Embodiments 44 to 144, wherein the subject has undergone previous treatment with SSRI/SNRI for at least one week prior to the induction phase.

Embodiment 146. The method according to any of Embodiments 44 to 145, wherein the subject is not suffering from bipolar disorder.

Embodiment 147. A pharmaceutical composition for the treatment of major depressive disorder in a human subject in need thereof, comprising:
(i) a therapeutic amount of dexmedetomidine or a pharmaceutically acceptable salt thereof,
(ii) a therapeutic amount of an anti-depressant and
(iii) one or more pharmaceutically acceptable carriers/excipients,
   wherein the composition is administered for about 1 day to about 28 days during the induction phase of the treatment cycle.

Embodiment 148. A pharmaceutical composition for the treatment of major depressive episode in a human subject in need thereof, comprising:
(i) a therapeutic amount of dexmedetomidine or a pharmaceutically acceptable salt thereof,
(ii) a therapeutic amount of an anti-depressant; and
(iii) one or more pharmaceutically acceptable carriers/excipients,
   wherein the composition is administered for about 1 day to about 28 days during the induction phase of the treatment cycle.

Embodiment 149. A pharmaceutical composition for the treatment or prevention of anxious distress in a human subject in need thereof, comprising:
(i) a therapeutic amount of dexmedetomidine or a pharmaceutically acceptable salt thereof;
(ii) a therapeutic amount of an SSRI/SNRI; and
(iii) one or more pharmaceutically acceptable carriers/excipients,
   wherein the composition is administered for about 1 day to about 28 days during the induction phase of the treatment cycle.

Embodiment 150. A pharmaceutical composition for the treatment or prevention of anxious distress caused by beginning treatment with a SSRI/SNRI in a human subject in need thereof, comprising:
(i) a therapeutic amount of dexmedetomidine or a pharmaceutically acceptable salt thereof;
(ii) a therapeutic amount of an SSRI/SNRI; and
(iii) one or more pharmaceutically acceptable carriers/excipients,
   wherein the composition is administered for about 1 day to about 28 days during the induction phase of the treatment cycle.

Embodiment 151. The pharmaceutical composition according to Embodiment 149 or 150, wherein administration of said composition is followed by maintenance phase comprising administration of a therapeutic amount of an SSRI/SNRI until the underlying disease (e.g., major depressive disorder) resolves or until the subject experiences a recurrence of anxious distress.

Embodiment 152. The pharmaceutical composition according to Embodiment to 150, wherein dexmedetomidine or a pharmaceutically acceptable salt thereof is dexmedetomidine hydrochloride.

Embodiment 153. The pharmaceutical composition according to any of Embodiments 149 or 150, wherein said SSRI is selected from the group consisting of sertraline, fluoxetine, norfluoxetine, fluvoxamine, citalopram, escitalopram, dapoxetine, paroxetine, femoxetine, vortioxetine, alaproclate and vilazodone or a pharmaceutically acceptable salt or ester or enantiomer thereof, preferably sertraline or escitalopram.

Embodiment 154 The pharmaceutical composition according to Embodiment 149 or 150, wherein said SNRI is selected from the group consisting of desvenlafaxine, venlafaxine, levomilnacipran, milnacipran, duloxetine, atomoxetine, Sibutramine, reboxetine, Tramadol or a pharmaceutically acceptable salt or ester or enantiomer thereof.

Embodiment 155. The pharmaceutical composition according to Embodiment 147 or 148, wherein the antidepressant is selected from the group consisting of selective serotonin reuptake inhibitors (SSRI's); selective serotonin and norepinephrine reuptake inhibitors (SNRI's); older tricyclic antidepressants (TCAs); monoamine oxidase inhibitors (MAO-inhibitors), reversible inhibitors of monoamine oxidase (RIMAs), atypical antidepressants; tertiary amine tricyclics and secondary amine tricyclic antidepressants.

Embodiment 156. The pharmaceutical composition according to Embodiments 147 to 155, wherein the subject is agitated.

Embodiment 157. The pharmaceutical composition according to Embodiments 147 to 155, wherein the subject is non-agitated.

Embodiment 158. The pharmaceutical composition according to Embodiments 149 to 157, wherein dexmedetomidine or a pharmaceutically acceptable salt thereof and the SSRI/SNRI are present in separate unit dosage forms.

Embodiment 159. The pharmaceutical composition according to Embodiment 158, wherein both unit dosage forms are administered to the subject by the same or different routes selected from oromucosal (sublingual or buccal or gingival), oral, topical, transdermal, intranasal or parenteral (intravenous, intramuscular, subcutaneous) routes.

Embodiment 160. The pharmaceutical composition according to embodiment 159, wherein said unit dosage forms are administered to the subject oromucosally in the form of tablet, film, spray, gel or drops.

Embodiment 161. The pharmaceutical composition according to Embodiment 160, wherein the dosage form is a film and the film is a thin film.

Embodiment 162. The pharmaceutical composition according to Embodiment 149 or 150, wherein the SSRI/SNRI is administered in a dosage form selected from the group comprising tablets, orally disintegrating tablets (ODTs), effervescent tablets, capsules, pellets, pills, lozenges, troches, films, powders, dispersible granules, cachets, aqueous solutions, syrups, emulsions, suspensions, solutions, soft gels, dispersions and the like.

Embodiment 163. The pharmaceutical composition according to Embodiment 162, wherein the SSRI/SNRI is administered orally as a tablet.

Embodiment 164. The pharmaceutical composition according to Embodiment 162, wherein the SSRI/SNRI is administered orally as capsule.

Embodiment 165. The pharmaceutical composition according to Embodiment 158, wherein dexmedetomidine or a pharmaceutically acceptable salt thereof is administered oromucosally in a dosage form selected from the group consisting of a film, wafer, patch, lozenge, gel, spray, tablet and liquid drops.

Embodiment 166. The pharmaceutical composition according to Embodiment 165, wherein dexmedetomidine or a pharmaceutically acceptable salt is administered oromucosally in the form of a film.

Embodiment 167. The pharmaceutical composition according to any of Embodiments 160 to 166, wherein the dosage forms are co-administered to the subject for about 28 days.

Embodiment 168. The pharmaceutical composition according to any of Embodiments 160 to 166, wherein the dosage forms are co-administered to the subject for about 21 days.

Embodiment 169. The pharmaceutical composition according to any of Embodiments 160 to 166, wherein the dosage forms are co-administered to the subject for about 14 days.

Embodiment 170. The pharmaceutical composition according to any of Embodiments 160 to 166, wherein the dosage forms are co-administered to the subject for about 7 days.

Embodiment 171. The pharmaceutical composition according to Embodiment 149 to 157, wherein dexmedetomidine or a pharmaceutically acceptable salt thereof and the SSRI/SNRI are present together in a single unit dosage form.

Embodiment 172. The pharmaceutical composition according to Embodiment 171, wherein said unit dosage form is administered to the subject through the oromucosal route in the form of tablet, film, spray, gel or drops.

Embodiment 173. The pharmaceutical composition according to Embodiment 172, wherein said unit dosage form is a film and the film is a thin film.

Embodiment 174. The pharmaceutical composition according to any of Embodiments 170 to 173, wherein the dosage form is administered to the subject for about 28 days.

Embodiment 175. The pharmaceutical composition according to any of Embodiments 170 to 173, wherein the dosage form is administered to the subject for about 21 days.

Embodiment 176 The pharmaceutical composition according to any of Embodiments 170 to 173, wherein the dosage form is administered to the subject for about 14 days.

Embodiment 177. The pharmaceutical composition according to any of Embodiments 170 to 173, wherein the dosage form is administered to the subject for about 7 days.

Embodiment 178. The pharmaceutical composition according to Embodiment 149 or 150, wherein the therapeutic amount of dexmedetomidine or a pharmaceutically acceptable salt thereof ranges from about 0.5 micrograms to about 240 micrograms.

Embodiment 179. The pharmaceutical composition according to Embodiment 149 or 150, wherein the therapeutic amount of dexmedetomidine or a pharmaceutically acceptable salt thereof is about 120 micrograms.

Embodiment 180. The pharmaceutical composition according to Embodiment 149 or 150, wherein the therapeutic amount of dexmedetomidine or a pharmaceutically acceptable salt thereof is about 180 micrograms.

Embodiment 181. The pharmaceutical composition according to Embodiment 149 or 150, wherein the therapeutic amount of the SSRI ranges from about 5 mg to about 250 mg.

Embodiment 182. The pharmaceutical composition according to Embodiment 149 or 150, wherein the therapeutic amount of the SNRI ranges from about 10 mg to about 500 mg.

Embodiment 183. The pharmaceutical composition according to any of Embodiments 147 to 182, wherein said dosage form(s) is administered once a day or multiple times a day.

Embodiment 184. The method or the pharmaceutical composition according to any of the preceding Embodiments, wherein the HAM-A score is improved or reduced in the subject compared to the score observed during start of treatment.

Embodiment 185. The method or the pharmaceutical composition according to any of the preceding Embodiments, wherein the HAM-D score is improved or reduced in the subject compared to the score observed during start of treatment.

Embodiment 186. The method or the pharmaceutical composition according to any of the preceding Embodiments, wherein the MADRS score is improved in the subject.

Embodiment 187. The method or the pharmaceutical composition according to any of the preceding Embodiments, wherein the ACES score is improved or reduced in the subject.

Embodiment 188. The method or the pharmaceutical composition according to any of the Embodiments 184 to 187, wherein the reduction in score is about 5% and about 100% (for example, about 5%, about 10%, about 15%, about 20%, about 25%, about 30%, about 35%, about 40%, about 45%, about 50%, about 55% or more), compared to baseline score observed prior to treatment.

Embodiment 189. The method or the pharmaceutical composition according to any of the preceding Embodiments, wherein the CGI-I score is improved in the subject.

Embodiment 190. The method or the pharmaceutical composition according to any of the preceding Embodiments, wherein the anxious distress is significantly reduced within 1 week (including within 1 day, 2 days, 3 days, 4 days, 5 days, 6 days, or 7 days).

Embodiment 191. The method or the pharmaceutical composition according to any of the preceding Embodiments, wherein the depression or the anxious distress is significantly reduced within 1 month, 2 months, 3 months, 6 months or 12 months.

Embodiment 192. An individual unit dosage form provided as a kit comprising the composition as described herein in a container with or without instructions for administration to a subject in need thereof.

Embodiment 193. A two-unit dosage form provided as a kit comprising the composition as described herein in one or more containers with or without instructions for the simultaneous, sequential or separate administration to a subject in need thereof.

Embodiment 194. The kit of Embodiment 192 or 193, comprises a package insert comprising instructions for using the compositions described herein for treatment of major depressive disorder in a subject.

Embodiment 195. The kit of Embodiment 192 or 193, comprises a package insert comprising instructions for using the compositions described herein for treatment of major depressive episode in a subject.

Embodiment 196. The kit of Embodiment 192 or 193, comprises a package insert comprising instructions for using the compositions described herein for treatment or prevention of anxious distress in a subject.

### EXAMPLES

### Example 1. To Evaluate the Effect of Dexmedetomidine Hydrochloride, Fluoxetine Hydrochloride and their Combination after Acute Intraperitoneal (i.p.) Administration Using the Elevated Plus-Maze Test in the Rats

### Test, Comparison, Reference and Control Substances

### Test Substance I

Name and vehicle: Dexmedetomidine Hydrochloride in physiological phosphate buffered saline.

### Test Substance II

Name and vehicle: Fluoxetine hydrochloride (Carbosynth reference BF163653), dispersed in 0.2% HPMC in physiological saline.

### Reference Substance

Name and vehicle: Clobazam (Carbosynth reference C-2991), dispersed in 0.2% HPMC in physiological saline.

### Control Substance: Vehicle for the Test Substance

Name: Phosphate Buffered Saline (PBS).

### Dosage Forms Preparation

Clobazam and Fluoxetine hydrochloride were dispersed in 0.2% HPMC in physiological saline using a mortar and a pestle.

Dexmedetomidine was dissolved in PBS under magnetic stirring. Doses were prepared fresh each day of the experiment W/V (stock) and then V/V (serial dilutions).

### Test Systems

Species used: Male Wistar (Han) rats, 180-280 g (max. range per experiment=500 g) at the beginning of the experiments.

Number: 52 males (including 2 spare). Additional spare animals were ordered, if needed.

Reason for SELECTION of Species: The characteristics of the animals used (age, strain, and species) were comparable with those described in the scientific literature.

In addition, historical data was maintained by Porsolt for tracking biological responses in positive and negative control groups over time for these standard tests and animals.

Breeder: Janvier Labs, 53940 Le Genest-Saint-Isle, France.

Receipt and ACCLIMATION PERIOD: Animals were delivered to the laboratory at least 7 days before the experiments during which time they were acclimatized to laboratory conditions.

Identification: Indelible marker on the tail.

Housing and Environmental Conditions: Rats were housed grouped in macrolon cages (no more than 5 animals per cage) on wood litter (SAFE, 89290 Augy, France). Environmental enrichment (such as tunnel, gnawing material and nesting material) were provided. The animal house was maintained under artificial lighting (12 hours) between 7:00 and 19:00 in a controlled ambient temperature of 22±2° C., and relative humidity between 30-70%.

Information related to any clinical signs and mortality was archived with the study materials.

Food and Water: All animals had a free access to food (Code A04-SAFE, 89290 Augy, France) and water.

Contaminant Analyses: The batches of diet and wood litter were analyzed by the suppliers for composition and contaminant levels. Bacterial and chemical analyses of water were performed regularly by external laboratories. These analyses included the detection of possible contaminants (pesticides, heavy metals and nitrates by-products).

It was expected that no contaminants that could interfere with, or prejudice, the outcome of the study were found in the diet, drinking water or wood litter.

### Experimental Design

### Treatment

### Route and Duration of Administration

The test, reference, comparison or vehicle formulations were administered as a single intraperitoneal dose (i.p.).

### Treatment Schedule

Dexmedetomidine, Fluoxetine and Clobazam were evaluated at 1-1 dose individually. The same dose of Dexmedetomidine was evaluated in combination with same dose of Fluoxetine as described in Table 1.

TABLE 1
Schedule of treatment

| | | | | | Administration |
|---|---|---|---|---|---|
| | Number | | | Concentration | Volume |
| Group | of rats | Treatment i.p. | Dose-level | (mg/mL) | (mL/kg) |
| 1 | 10 males | Control Substance | 0 | 0 | 5 |
| 2 | 10 males | Dexmedetomidine | 10 μg/kg | 0.002 | 5 |
| 3 | 10 males | Fluoxetine | 32 mg/kg | 6.4 | 5 |
| 4 | 10 males | Fluoxetine + | 32 mg/kg + | 6.4 + 0.002 | 5 |
| | | Dexmedetomidine | 10 μg/kg | | |
| 5 | 10 males | Clobazam | 16 mg/kg | 3.2 | 5 |

### Experimental Procedure

### Elevated Plus-Maze Test in the Rat

The method, which detects anxiolytic activity, followed that described by Handley and Mithani (Naunyn. Schmied. Arch. Pharmacol., 327, 1-5, 1984).

Under standard artificial lighting (˜200 Lux), there was a contrast between the closed (obscure and safe) arms and the open (brightly illuminated and anxiogenic) arms of the maze. In these conditions, control rats avoid the open arms whereas anxiolytics increase exploratory activity in the open arms.

The day preceding the experiment, rats were randomized based on their body weight.

The maze consisted of 4 arms of equal length and width (50×10 cm) arranged in the form of a plus sign (+). Two opposite arms were enclosed by 40 cm high walls (closed arms). The 2 other arms had no walls (open arms). The maze was raised approximately 65 cm above the floor. A rat was placed in the centre of the plus-maze and left to explore for 5 minutes.

The test was video-recorded and the number of entries into the open and closed arms and the time spent on the open arms were measured automatically using a video tracking system (EthoVision, Noldus). The % of open arm entries (open arm entries/total arm entries×100) were calculated. The total distance travelled was also reported.

10 rats were studied per group. The test was performed blind over 2 days with half of animals in each group each day.

Dexmedetomidine was evaluated at a dose of 10 μg/kg, administered i.p. 30 minutes before the test, and compared with a vehicle control group.

Fluoxetine (32 mg/kg i.p.) administered 30 minutes before the test was used as comparison substance.

Clobazam (16 mg/kg i.p.), administered 30 minutes before the test was used as reference substance.

All animals received 2 consecutive administrations at 30 minutes before the test. When a drug was not due, animals received administration of vehicle as described below in table 2:

TABLE 2
Treatment given 30 mins before the test

| Group | Treatment i.p. - 30 min | Treatment i.p. -30 min |
|---|---|---|
| no. | before the test | before the test |
| 1 | PBS | 0.2% HPMC in saline |
| 2 | Dexmedetomidine | 0.2% HPMC in saline |
| | 10 μg/kg | |
| 3 | PBS | Fluoxetine 32 mg/kg |
| 4 | Dexmedetomidine | Fluoxetine 32 mg/kg |
| | 10 μg/kg | |
| 5 | PBS | Clobazam 16 mg/kg |

Data (for each parameter measured) with the test substance was analysed by comparing treated groups with vehicle controls using one-way ANOVA followed by post-hoc Dunnett's tests.

Data with the comparison reference substance (clobazam) was separately analysed by comparing each treated group with vehicle controls using unpaired Student's T-tests.

Results and Discussion: The results of this study are presented in Tables 3 to 6 as below.

TABLE 3a

| Group. | | Number of entries |
|---|---|---|
| No. | Treatment | in the open arm |
| 1 | Vehicle (i.p.) | 2.10 ± 0.80 |
| 2 | Dexmedetomidine (10 μg/kg | 3.10 ± 0.50 |
| | i.p.) | |
| 3 | Fluoxetine (32 mg/kg i.p.) | 0.40 ± 0.20 |
| 4 | Dexmedetomidine (10 μg/kg | 1.00 ± 0.30 |
| | i.p.) + Fluoxetine | |
| | (32 mg/kg i.p.) | |
| 5 | Clobazam (16 mg/kg i.p.) | 7.80 ± 0.80 |

TABLE 3b

| Group | | Percentage of entries |
|---|---|---|
| No. | Treatment | in the open arm |
| 1 | Vehicle (i.p.) | 18.50 ± 5.00 |
| 2 | Dexmedetomidine (10 μg/kg | 35.20 ± 6.20 |
| | i.p.) | |
| 3 | Fluoxetine (32 mg/kg i.p.) | 3.30 ± 3.30 |
| 4 | Dexmedetomidine (10 μg/kg | 13.80 ± 5.90 |
| | i.p.) + Fluoxetine | |
| | (32 mg/kg i.p.) | |
| 5 | Clobazam (16 mg/kg i.p.) | 54.90 ± 6.70 |

TABLE 4

| Group | | Time spent in |
|---|---|---|
| No. | Treatment | the open arm |
| 1 | Vehicle (i.p.) | 17.10 ± 6.30 |
| 2 | Dexmedetomidine (10 μg/kg | 24.10 ± 4.40 |
| | i.p.) | |
| 3 | Fluoxetine (32 mg/kg i.p.) | 6.50 ± 4.70 |
| 4 | Dexmedetomidine (10 μg/kg | 13.10 ± 5.00 |
| | i.p.) + Fluoxetine | |
| | (32 mg/kg i.p.) | |
| 5 | Clobazam (16 mg/kg i.p.) | 111.80 ± 28.50 |

TABLE 5

| | | Total number |
|---|---|---|
| Group | | of entries in |
| No. | Treatment | the four arms |
| 1 | Vehicle (i.p.) | 10.00 ± 1.60 |
| 2 | Dexmedetomidine (10 μg/kg | 9.70 ± 1.60 |
| | i.p.) | |
| 3 | Fluoxetine (32 mg/kg i.p.) | 4.30 ± 0.60 |
| 4 | Dexmedetomidine (10 μg/kg | 5.50 ± 1.30 |
| | i.p.) + Fluoxetine (32 | |
| | mg/kg i.p.) | |
| 5 | Clobazam (16 mg/kg i.p.) | 15.80 ± 2.20 |

TABLE 6

| | | Total distance |
|---|---|---|
| Group. | | travelled in |
| No. | Treatment | the four arms |
| 1 | Vehicle (i.p.) | 1203.30 ± 160.40 |
| 2 | Dexmedetomidine (10 μg/kg | 1080.70 ± 135.90 |
| | i.p.) | |
| 3 | Fluoxetine (32 mg/kg i.p.) | 419.00 ± 66.40 |
| 4 | Dexmedetomidine (10 μg/kg | 544.30 ± 104.90 |
| | i.p.) + Fluoxetine (32 | |
| | mg/kg i.p.) | |
| 5 | Clobazam (16 mg/kg i.p.) | 1487.10 ± 223.90 |

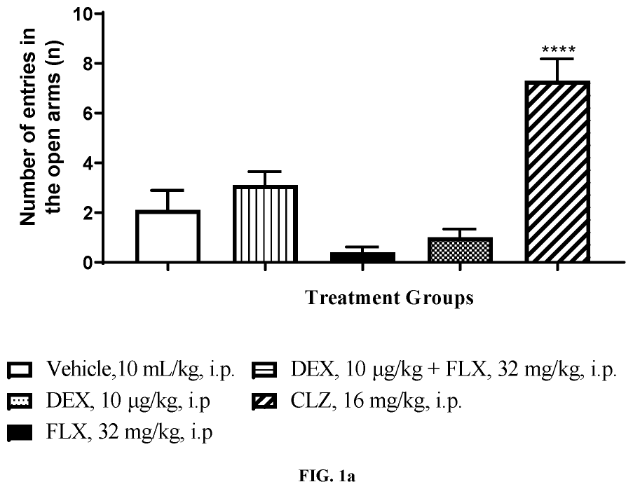

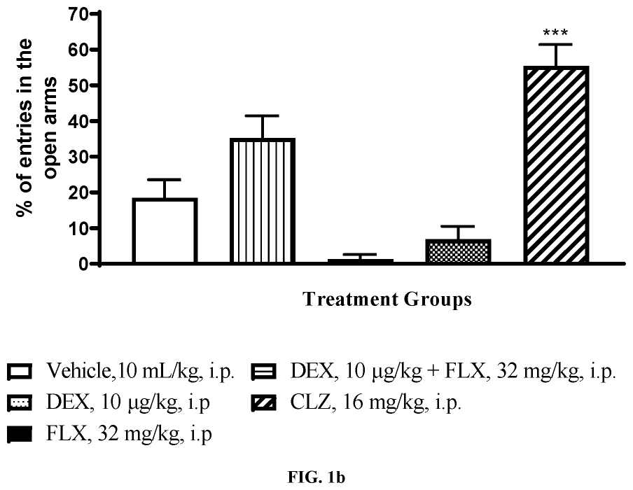

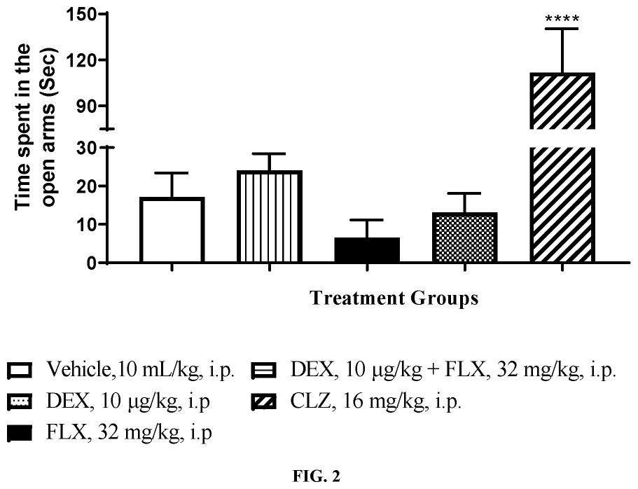

Discussion—On administration of Dexmedetomidine; 10 μg/kg, i.p. (Group 2), there was increase in the number of entries in open arms (FIG. 1a and Table 3a); % of entries (FIG. 1b and Table 3b), or the time spent (FIG. 2, Table 4) in the open arms of the elevated plus-maze, but results were not significant as compared with vehicle control (Group 1).

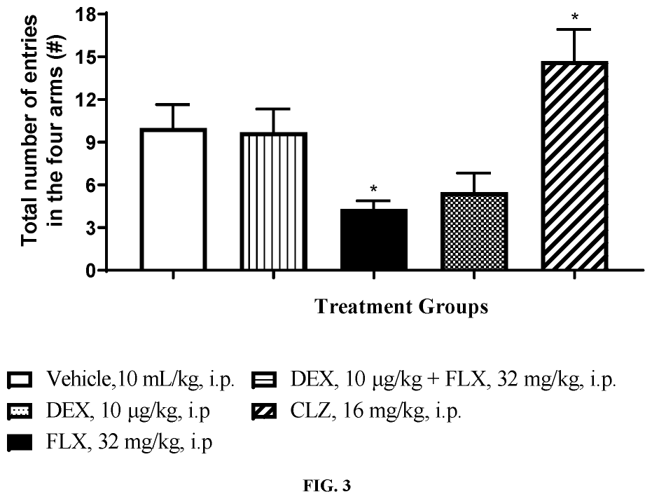

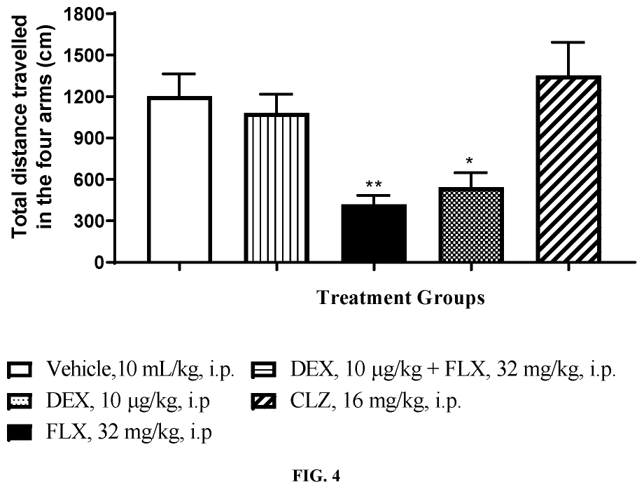

Fluoxetine 32 mg/kg; i.p. treated group (Group 3), decreased the number of entries (FIG. 1a and Table 3a), the % of entries (FIG. 1b and Table 3b) and the time spent (FIG. 2; Table 4) in the open arms of the elevated plus-maze as compared with vehicle control (Group 1) but the result was not significant. In addition, it significantly decreased the total number of entries and the total distance travelled in the four arms of the maze (−57%, p<0.05; FIG. 3 and −65%, p<0.01; FIG. 4), as compared with vehicle control (Group 1).

When Fluoxetine (32 mg/kg) co-administered with Dexmedetomidine 10 μg/kg (Group 4), the synergistic effect increases the number of entries (FIG. 1a), the % of entries (FIG. 1b) and the time spent (FIG. 2, Table 4) in the open arms of the elevated plus-maze as compared to Fluoxetine 32 mg/kg alone (Group 3) (+150%, +431% and +102% NS, respectively). There were no significant effects on the total number of entries as compared to Fluoxetine alone (+28%, NS FIG. 3, Table 5). However, the total distance travelled decreased significantly as compared to vehicle control (−55%, p<0.05; FIG. 4, Table 6), but not significantly compared to Fluoxetine alone (+29%, NS; FIG. 4, Table 6).

These results suggested the absence of anxiolytic-like activity for Dexmedetomidine at g/kg i.p. in the Elevated Plus Maze Test in the rat. On the other hand, Fluoxetine at 32 mg/kg i.p. tended to show anxiogenic like effect. Dexmedetomidine at 10 μg/kg tended to reverse the anxiogenic-like effect of Fluoxetine (32 mg/kg i.p.).

### Example 2. To Evaluate the Effect of Dexmedetomidine Hydrochloride, Fluvoxamine Maleate and their Combination after Acute Intraperitoneal (i.p.) Administration Using the Elevated Plus Maze Test in Yohimbine Challenged Rats

### Experimental Groups:

108 male Wistar rats were used. They were randomly distributed to 8 different experimental groups (12 animals per group) as given in Table 7:

TABLE 7
Details of the groups

| | | | | | Time |
|---|---|---|---|---|---|
| | | | | | before |
| | | | | | trial |
| Gp # | Description | n | route | Dosage volume | (min) |
| 1 | Vehicle (Saline)/Vehicle | 12 | i.p./i.p. | 1 ml/kg-1 ml/kg | 30/60 |
| | (Saline) | | | | |
| 2 | Yohimbine (2.5 mg/kg; i.pa)/ | 12 | i.p./i.p. | 1 ml/kg-1 ml/kg | 30/60 |
| | Vehicle | | | | |
| 3 | Yohimbine (2.5 mg/kg; i.p)/ | 12 | i.p./i.p. | 1 ml/kg-1 ml/kg | 30/30 |
| | Diazepam 1 mg/kg | | | | |
| 4 | Yohimbine (2.5 mg/kg; i.p)/ | 12 | i.p./i.p. | 1 ml/kg-1 ml/kg | 30/60 |
| | Dexmedetomidine 10 μg/kg | | | | |
| 5 | Yohimbine (2.5 mg/kg; i.p)/ | 12 | i.p./i.p. | 1 ml/kg-1 ml/kg | 30/60 |
| | Fluvoxamine 3 mg/kg | | | | |
| 6 | Yohimbine (2.5 mg/kg; i.p)/ | 12 | i.p./i.p. | 1 ml/kg-1 ml/kg | 30/60 |
| | Fluvoxamine 10 mg/kg | | | | |
| 7 | Yohimbine (2.5 mg/kg; i.p)/ | 12 | i.p./i.p./i.p. | 1 ml/kg-1 ml/kg- | 30/60/60 |
| | Fluvoxamine 3 mg/kg/ | | | 1 ml/kg | |
| | Dexmedetomidine 10 μg/kg | | | | |
| 8 | Yohimbine (2.5 mg/kg; i.p)/ | 12 | i.p./i.p./i.p. | 1 ml/kg-1 ml/kg- | 30/60/60 |
| | Fluvoxamine 10 mg/kg/ | | | 1 ml/kg | |
| | Dexmedetomidine 10 μg/kg | | | | |

Treatment with Test Compounds:
As outlined in the table
Vehicle is Saline

### Elevated Plus Maze:

After compound administration, the rat was placed on the platform opposite a closed arm. The number of entries and the time spent in each arm were recorded during a 5 min period (deliverables). The animal was considered as entered in arm when it placed its four paws into the arm.

### Series and Data:

The number of rats processed per day was about 20 in this model. Therefore, the experiment was split in several series.

### Statistical Analysis:

Analysis of variance (ANOVA) was performed on the result data. Fisher's PLSD was used for

pairwise comparisons and p value <0.05 was considered significant.

Results and Discussion: The results of this study are presented in below Tables 8 and Table 9.

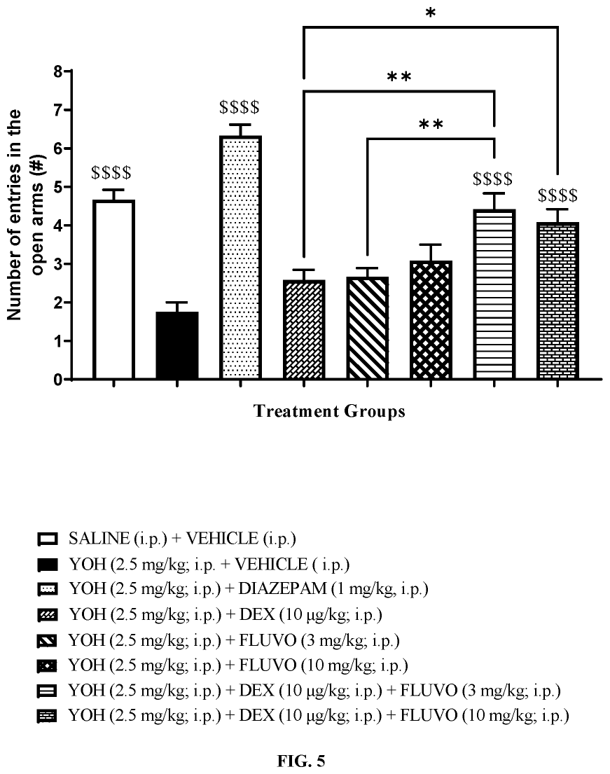

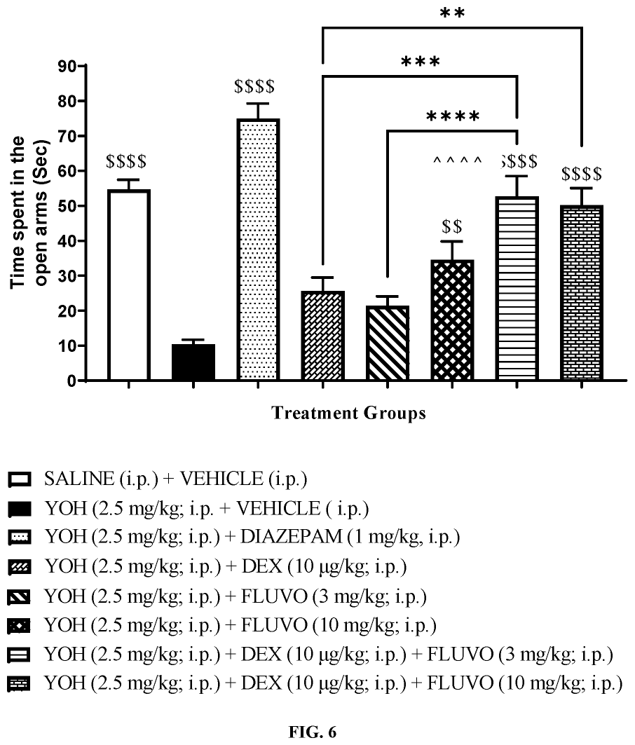

Yohimbine (2.5 mg/kg; i.p.) administered vehicle group (Group 2) showed a significant decrease in the number of entries (Table 8, FIG. 5, −63%, p<0.0001) to and the time spent in the open arms (Table 9, FIG. 6, −81%, p<0.0001) as compared to vehicle (saline) treated group (Group 1). This suggested the occurrence of Yohimbine-induced anxiety-like behavior as assessed in the EPM paradigm.

The group treated with diazepam 1 mg/kg; i.p. (Group 3) reversed Yohimbine-induced anxiety as shown by the significant increase in the number of open arm entries (Table 8, FIG. 5, +262%, p<0.0001) as well as the time spent (Table 9, FIG. 6, +620%, p<0.0001) in the open arms as compared with Yohimbine administered vehicle group (Group 2).

Dexmedetomidine (10 μg/kg; i.p.) treated group (Group 4), although showed an increase in number of open arm entries (+48%, NS) and time spent in the open arms (+146%, NS) of the elevated plus-maze but the results were not found to be significant compared with Yohimbine-administered vehicle group (Group 2).

Further, no significant improvement in terms of the number of entries in the open arms (FIG. 5) were observed in Groups 5 and 6 that were treated with Fluvoxamine 3 mg/kg and Fluvoxamine 10 mg/kg; i.p. respectively when compared with Yohimbine-administered vehicle group (Group 2). Moreover, Fluvoxamine (3 mg/kg; i.p) treated group (Group 5) did not significantly increased the time spent in the open arms (FIG. 6) as compared with Yohimbine-administered vehicle group (+105%, NS). However, Fluvoxamine (10 mg/kg; i.p.) treated group (Group 6) significantly increased the time spent in the open arms (FIG. 6) as compared with Yohimbine-administered vehicle group in Group 2 (+232%, p<0.01).

The co-administration of Dexmedetomidine (10 μg/kg; i.p.) with Fluvoxamine (3 mg/kg; i.p.) and with Fluoxamine (10 mg/kg; i.p.) (as in groups 7 and 8) respectively significantly increased the number of entries (FIG. 5, +152%, p<0.0001 and +133%, p<0.0001, respectively) to and the time spent (FIG. 6, +406%, p<0.0001 and +382%, p<0.0001, respectively) in the open arms as compared with Yohimbine-administered vehicle group (Group 2). Additionally, Group 7 treated with combination of Dexmedetomidine (10 μg/kg) with Fluvoxamine (3 mg/kg) significantly increased the number of entries (FIG. 5, +71%, p<0.01 when compared with individually treated i.e. Dexmedetomidine; 10 μg/kg (group 4) and +66%, p<0.01) and Fluvoxamine; 3 mg/kg (Group 5).

Group 7 (Dex 10 μg/kg+Fluvoxamine 3 mg/kg) also significantly increased the time spent in the open arms of the maze (FIG. 6, +105%, p<0.001) compared to individually treated Dexmedetomidine; 10 μg/kg (group 4) and +146%, p<0.0001) and Fluvoxamine; 3 mg/kg (group 5). Co-administration of Dexmedetomidine; 10 μg/kg) with Fluvoxamine; 10 mg/kg (Group 8) significantly increased the number of entries (FIG. 5, +58%, p<0.05 when compared with individually treated Dexmedetomidine; 10 μg/kg (Group 4), however when compared with Fluvoxamine (10 mg/kg) alone (Group 6), results were insignificant (+32%, NS vs). Similar trend was observed in the time spent in open arms as the combination group 8 (Dexmedetomidine (10 μg/kg) with Fluvoxamine (10 mg/kg) (FIG. 6, +96%, p<0.01) showed significant result compared with individual Dexmedetomidine (10 μg/kg) alone (Group 4) but non-significant w.r.t Fluvoxamine 10 mg/kg alone (+45%, NS) in Group 6.

The above results suggested that though there is anxiolytic-like effect of Dexmedetomidine (10 μg/kg i.p.) alone and Fluvoxamine (3 mg/kg i.p) alone in the Yohimbine administered rats, the observed effect was not statistically significant (See groups 4 and 5 vs group 2). On the other hand, Fluvoxamine at 10 mg/kg i.p. showed significant anxiolytic-like effect in terms of the time spent in the open arms. Combination of Dexmedetomidine, 10 μg/kg and Fluvoxamine, 3 mg/kg (Group 7) showed a significant anxiolytic-like effect in Yohimbine administered rats in elevated plus maze test not only with respect to the Yohimbine administered group but also with respect to its individual drug treatment groups.

Considering that the anxiolytic-like effect of Dexmedetomidine (10 μg/kg) and Fluvoxamine (3 mg/kg), individually (in group 4 and group 5) in terms of number of entries and time spent in open arms are not significant, the very high level of statistical significance shown by this combination (Table 10), indicates a synergistic effect by these two drugs at this dose combination (group 7). The anxiolytic-like effects of the combination (group 7) were much higher than the additive sum of their individual effects of Dexmedetomidine (10 μg/kg; Group 4) and Fluvoxamine (3 mg/kg, group 5) (+152% for the number of entries and +406% for the time spent in the open arms, Table 10).

TABLE 8

| | | Number of entries |
|---|---|---|
| Group | | in the open arms |
| No. | Treatment Groups | (Mean ± SEM) |
| 1 | SALINE (i.p.) + | 4.67 ± 0.26 |
| | VEHICLE (i.p.) | |
| 2 | YOH (2.5 mg/kg; i.p. + | 1.75 ± 0.25 |
| | VEHICLE (i.p.) | |
| 3 | YOH (2.5 mg/kg; i.p.) + | 6.33 ± 0.28 |
| | DIAZEPAM (1 mg/kg, i.p.) | |
| 4 | YOH (2.5 mg/kg; i.p.) + | 2.58 ± 0.26 |
| | DEX (10 μg/kg; i.p.) | |
| 5 | YOH (2.5 mg/kg; i.p.) + | 2.67 ± 0.22 |
| | FLUVO (3 mg/kg; i.p.) | |
| 6 | YOH (2.5 mg/kg; i.p.) + | 3.08 ± 0.42 |
| | FLUVO (10 mg/kg; i.p.) | |
| 7 | YOH (2.5 mg/kg; i.p.) + | 4.42 ± 0.42 |
| | DEX (10 μg/kg; i.p.) + | |
| | FLUVO (3 mg/kg; i.p.) | |
| 8 | YOH (2.5 mg/kg; i.p.) + | 4.08 ± 0.34 |
| | DEX (10 μg/kg; i.p.) + | |
| | FLUVO (10 mg/kg; i.p.) | |

TABLE 9

| | | Time spent in |
|---|---|---|
| Group | | the open arms |
| No. | Treatment Groups | (Mean ± SEM) |
| 1 | SALINE (i.p.) + | 54.68 ± 2.78 |
| | VEHICLE (i.p.) | |
| 2 | YOH (2.5 mg/kg; i.p. + | 10.42 ± 1.28 |
| | VEHICLE (i.p.) | |
| 3 | YOH (2.5 mg/kg; i.p.) + | 74.98 ± 4.31 |
| | DIAZEPAM (1 mg/kg, i.p.) | |
| 4 | YOH (2.5 mg/kg; i.p.) + | 25.66 ± 3.84 |
| | DEX (10 μg/kg; i.p.) | |
| 5 | YOH (2.5 mg/kg; i.p.) + | 21.39 ± 2.67 |
| | FLUVO (3 mg/kg; i.p.) | |
| 6 | YOH (2.5 mg/kg; i.p.) + | 34.60 ± 5.24 |
| | FLUVO (10 mg/kg; i.p.) | |
| 7 | YOH (2.5 mg/kg; i.p.) + | 52.72 ± 5.79 |
| | DEX (10 μg/kg; i.p.) + | |
| | FLUVO (3 mg/kg; i.p.) | |
| 8 | YOH (2.5 mg/kg; i.p.) + | 50.19 ± 4.88 |
| | DEX (10 μg/kg; i.p.) + | |
| | FLUVO (10 mg/kg; i.p.) | |

Table 10 shows the percentage change in the effect of treatment of Dexmedetomidine (10 μg/kg; i.p.), Fluvoxamine (3 and 10 mg/kg; i.p.) and the combination of Dexmedetomidine (10 μg/kg; i.p.) with Fluvoxamine (3 and 10 mg/kg; i.p.) in rats with Yohimbine-induced anxiety on the number of entries and the time spent in the open arm in Elevated plus maze as compared to Yohimbine administered group.

TABLE 10

| | | In the open arms | |
|---|---|---|---|
| | | Percentage change compared to | |
| Group. | | Yohimbine administered group | |
| No. | Treatment groups | Number of entries | Time spent |
| 1 | YOH (2.5 mg/kg) + | +48% | +146% |
| | DEX (10 μg/kg) | | |
| 2 | YOH (2.5 mg/kg) + | +52% | +105% |
| | FLUVO (3 mg/kg) | | |
| 3 | YOH (2.5 mg/kg) + | +76% | +232% |
| | FLUVO (10 mg/kg) | | |
| 4 | YOH (2.5 mg/kg) + | +152% | +406% |
| | DEX (10 μg/kg) + | | |
| | FLUVO (3 mg/kg) | | |
| 5 | YOH (2.5 mg/kg) + | +133% | +382% |
| | DEX (10 μg/kg) + | | |
| | FLUVO (10 mg/kg) | | |

Conclusion—The combined anxiolytic-like effect of Dexmedetomidine (10 μg/kg) and Fluvoxamine (3 mg/kg), has been found to be more than the additive sum of their individual effects with respect to both the parameters measured in the Elevated Plus Maze.

### Example 3: Phase-1 Study for Maximum Tolerated Dose Finding Study for Dexmedetomidine Hydrochloride Sublingual Film with Concomitant Treatment with an Antidepressant in Healthy Volunteers

### Primary Objective

The primary objective is to first determine the maximally tolerated dose (MTD) of dexmedetomidine sublingual film versus placebo alone in healthy volunteers and then to determine if this MTD of dexmedetomidine sublingual film is adequately tolerated when given with an effective dose of a serotonin and norepinephrine reuptake inhibitor (SNRI; duloxetine) that incorporates the acute pharmacological actions of both a selective serotonin reuptake inhibitor (SSRI) and a norepinephrine reuptake inhibitor (NRI).

### Secondary Objectives

The key secondary objective is to assess the safety and tolerability of each dose studied. The specific safety matters of interest are: sedation, any occurrence of potentially clinically significant changes in either resting hemodynamic vital sign values or hemodynamic vital sign values obtained at rest and under orthostatic stress.

### Other Secondary Objective

To characterize the pharmacokinetics (PK) of dexmedetomidine sublingual film and its metabolites in healthy volunteers after single and multiple doses.

### Primary Endpoints

Determine the safety profile of dexmedetomidine sublingual film as measured by vital signs, the Agitation-Calmness Evaluation Scale (ACES) administered after every morning dose of study treatment (on Days 1, 4, and 7) for excessive sedation, hemodynamic changes in blood pressure and heart rate (both at rest and under orthostatic stress), and reports of adverse events (AEs) for sedation, hemodynamic changes, and other events.
Describe the overall tolerability in terms of AE reports and local site (oral/sublingual) tolerability of the oral film.

### Secondary Endpoints

Pharmacokinetics: The plasma concentration-time data is analyzed by standard non-compartmental methods to estimate the pharmacokinetic (PK) parameters which include, maximum plasma concentration (Cmax), the time at which the Cmax is observed (Tmax), areas under the curve (AUCs; partials and infinity), half-life, apparent clearance, and apparent volume of distribution.

### Study Design

### Overall Study Design and Plan

This is a randomized, double-blind, placebo-controlled, MAD study assessing the MTD of dexmedetomidine sublingual film in healthy volunteers and subsequently when the MTD is administered concomitantly with a SNRI antidepressants in a total of 6 cohort groups.

A total of 102 subjects (i.e., healthy volunteers) are planned to be enrolled at 1 study site in the US. These subjects are enrolled in one of six cohorts; the first five cohorts consisted of 18 subjects each, and the final cohort consists of 12 subjects.

This inpatient study randomizes subjects 2:1 to receive dexmedetomidine sublingual film or matching placebo for 7 days. The dose of dexmedetomidine sublingual film received is dependent on the cohort that a given subject is assigned to as follows:
Cohort 1: 30 μg or placebo (12 active; 6 placebo) administered every morning (qAM).
Cohort 2: 60 μg or placebo (12 active; 6 placebo) administered qAM.
Cohort 3: 80 μg (12 active; 6 placebo) administered qAM.
Cohort 4: 30 μg administered qAM and 60 μg administered every evening (qPM), or placebo (12 active; 6 placebo).
Cohort 5: 40 μg administered qAM and 80 μg administered qPM, or placebo (12 active; 6 placebo)

Cohort 6: 30 milligrams (mg) duloxetine twice daily (BID) in addition to the MTD of dexmedetomidine sublingual film that has been identified without duloxetine or placebo (8 active; 4 placebo). This dose of dexmedetomidine sublingual film can be 30 μg qAM; 60 μg qAM, 80 μg qAM, 30 μg qAM and 60 μg qPM, or 40 μg qAM and 80 μg qPM. The determination of the MTD is determined after all potential dexmedetomidine sublingual film or placebo cohorts have been treated and the safety data has been reviewed. Subjects in this cohort are treated for 7 days with comparable evaluations conducted as with the 3 initial cohorts, above.

Following the completion of each cohort, a review of the safety data is conducted by the sponsor and the Investigator to determine if there are any safety signals prior to initiation of the following cohort. If any such signals are noted during the review, dose escalation is halted, the dose administered in the previous cohort is considered the MTD, and the study is immediately move to cohort 6. If any such signals are noted during the review of cohort 1, the study was terminated.

Upon confirmation of eligibility, subjects are admitted to the inpatient unit the afternoon before the first day on which they receive treatment. Subjects are observed and assessed for an additional 3 days following the last dose of study treatment on the inpatient unit with the primary purpose of this observation period being the assessment of possible withdrawal/rebound effects. For subjects discontinuing the study early, they are encouraged to remain in the inpatient facility for the 3 days of post study drug observation and assessments.

Upon confirmation of eligibility, subjects are randomized 2:1 to receive dexmedetomidine sublingual film or matching placebo, starting with Cohort 1 (30 μg), and moving to subsequent dosing cohorts after safety review when all participants complete 7 days of treatment with dexmedetomidine sublingual film in the preceding cohort and all safety data for those 7 days has been thoroughly reviewed (including the data from 3 days of assessments following the last dose of study treatment). Duration of treatment per cohort is 7 days. During this study, subjects are domiciled in an inpatient clinical research setting/facility (admitted the afternoon of the day before they receive the first dose of study treatment). Subjects are discharged from the facility on the third day after receiving their last dose of study treatment (and duloxetine for those in cohort 6) following their final safety assessment.

At the time of study treatment dosing as described above, subjects are instructed on how to take the investigational product sublingually, and that they should retain the investigational product in the sublingual cavity until dissolved. The subject self-administered dexmedetomidine sublingual film. If the subject is unable to self-administer, the event is recorded, and the subject's participation is concluded.

Participants are also evaluated for local irritation around the area where the film is placed. Safety assessments are conducted periodically before and after dosing as per the Schedule of Events for each potential cohort (see Table 11 and Table 12). All efforts are made to have all assessments completed as per protocol.

Safety and tolerability are measured throughout the treatment period at various timepoints as described in the Schedule of Events in Table 11.

Dense sampling of plasma concentrations of dexmedetomidine sublingual film and its metabolites are conducted in each cohort. Blood samples are drawn pre-dose and at 0.25, 0.50, 1.00, 2.00, 3.00, 4.00, 6.00, 8.00, 10.00, 12.00, and 24.00 hours post dose on Days 1, 2, and 7. For those cohorts receiving BID administration, blood samples for the assays are collected on a schedule identical to the above schedule following the morning dose. In addition, blood samples are drawn pre-dose on Days 3-6 for both the once daily and twice daily dosing. These blood samples are obtained for assaying the plasma concentrations of dexmedetomidine sublingual film and its metabolites.

### Subject Population

### Selection of Study Population

102 maximum subjects (i.e., healthy volunteers) are planned to be enrolled at 1 study site in the US. Subjects who do not complete the full 7 days of treatment and 3 days of follow-up observation are replaced.

### Inclusion Criteria

A subject is eligible for inclusion in the study if he or she meets all of the following criteria:
1. Subject is a healthy volunteer, aged 18 or older.
2. Subject is willing to provide informed consent.
3. Body Mass Index (BMI) is between 18.5-29.9.
4. Subjects who are in good general health prior to study participation as determined by a detailed medical history, physical examination, a triplicate set of 10 second 12-lead electrocardiogram (ECG), blood chemistry profile, hematology, urinalysis, and in the opinion of the Principal Investigator or designee.
5. If the subject is female, she must not be pregnant or breastfeeding. If she is of childbearing potential, she must agree to use a clinically acceptable method of contraception or be abstinent during the study and 1 month following completion of the study.

### Exclusion Criteria

A subject is excluded from the study if he or she meets any of the following criteria:
1. Use of benzodiazepines or other sedative-hypnotic drugs during the study.
2. Use of any antipsychotic within 1 week (6 weeks for a depot antipsychotic) of beginning the study or use of an antipsychotic during the study.
3. Subjects with an alcohol or substance use disorder clinically considered to need treatment.
4. Treatment with alpha-1 noradrenergic blockers (terazosin, doxazosin, tamsulosin, alfuzosin, or prazosin) or other prohibited medications.
5. Subjects with an abnormal score on the Columbia-Suicide Severity Rating Scale (C-SSRS) must be excluded (C-SSRS≥4).
6. Female subjects who have a positive pregnancy test at screening or at baseline (Day 0), or are breastfeeding.
7. Subjects who have hydrocephalus, seizure disorder, or history of significant head trauma, stroke, transient ischemic attack, subarachnoid bleeding, brain tumor, encephalopathy, meningitis, Parkinson's disease, or focal neurological findings.
8. History of syncope or other syncopal attacks, current evidence of hypovolemia, orthostatic hypotension (a systolic blood pressure (SBP) decrease of at least 20 mmHg or a diastolic blood pressure (DBP) decrease of at least 10 mmHg within 3 minutes of standing) at screening and baseline, a screening and baseline heart rate of <60 beats per minutes, or systolic blood pressure (SBP)<100 mmHg or diastolic blood pressure (DBP)<55 mmHg at screening and at baseline.
9. Subjects with laboratory or ECG abnormalities considered clinically significant by the investigator or qualified designee [Advanced heart block (second-degree or above atrioventricular block without pacemaker), diagnosis of Sick sinus syndrome] that would have clinical implications for the subject participation in the study.
10. Subjects with personal or known family history of genetic long-QT syndrome (if known family history but subject had genetic confirmation of lack of the familial genetic alteration, the subject can be entered). The average of the 3 screening QTcF values >470 milliseconds (msec) for female subjects and >450 msec for male subjects.
11. Subjects with serious or unstable medical illnesses. These include current hepatic (moderate-severe hepatic impairment), renal, gastroenterological, respiratory, cardiovascular (including ischemic heart disease, congestive heart failure), endocrinologic, or hematologic disease.
12. Subjects who have received an investigational drug within 30 days prior to first dose of study drug.
13. Subjects who are considered by the investigator, for any reason, to be an unsuitable candidate for receiving DEX (dexmedetomidine sublingual film); e.g., subjects with a history of allergic reactions to DEX.
14. Use of any medication that could interfere with the activities of uridine diphosphate-glucuronosyltransferases (UGT) enzymes and cytochrome P450 2A6 (CYP2A6) within 2 weeks of beginning the study until study completion.
15. Subjects who have a positive drug screen at screening or baseline (Day 0).

Upon confirmation of eligibility, subjects will be randomized 2:1 to receive dexmedetomidine hydrochloride sublingual film (at the dose specified per cohort) or matching placebo. Subjects withdrawn from the study retain their randomization number, if already given.

### Treatment Administration

At the time of study treatment dosing as described above, subjects are instructed on how to take the investigational product sublingually, and that they should retain the investigational product in the sublingual cavity until dissolved. The subject self-administered the sublingual film of dexmedetomidine hydrochloride. If the subject is unable to self-administer, the event is recorded, and the subject's participation is concluded. The subject should be restricted from food or water intake for 15 minutes post dose.

Participants are also evaluated for local irritation around the area where the film is placed on a daily basis during both the treatment and follow-up periods of the study.

Subjects in Cohort 6 are also be instructed by the site staff on how to take their dose of duloxetine.

### Study Assessments

### Pharmacokinetics

Blood samples (4 milliliters (mL)) are collected per the Schedule of Events (Table 11 and Table 12).

For each subject, up to 80 blood samples (up to 320 mL of blood) is collected during the study for PK analysis. Subjects in cohorts 1-3 have 40 samples taken. Subjects in cohorts 4 and 5 had 80 samples taken. Subjects in cohort 6 have 40 or 80 samples taken; the total number depend on whether subjects receive only qAM dosing or BID dosing of dexmedetomidine sublingual film in that cohort.

### Statistical Methods

### General Considerations

A Statistical Analysis Plan (SAP) that describes the details of the analyses to be conducted is finalized before database lock.

### Statistical Analyses

Data is summarized by treatment using descriptive statistics (number of subjects, mean, median, standard deviation, minimum, and maximum) for continuous variables and summarized by treatment using frequencies and percentages for categorical variables. Baseline is defined as the last observation available prior to initiation of study medication.

### Safety Analyses

All safety analyses are performed using the Safety Population. All subjects who receive at least one dose of study drug are included in the population for safety analysis.

Adverse events are characterized by type, severity, seriousness, and relationship to treatment. Adverse events are coded by preferred term and system organ class using the most current version of MedDRA. Incidence of AEs is summarized by treatment overall, by severity, and by relationship to study drug. Serious adverse events and AEs leading to discontinuation of study drug is also be presented. Listings of subjects who experience withdrawal due to an AE, serious AEs and/or death is presented.

Laboratory parameters are summarized by treatment using descriptive statistics and data listings of clinically significant abnormalities. Vital signs, pulse oximetry, and ECG data is summarized by changes from baseline values using descriptive statistics.

Safety evaluation is based on the incidence of treatment-emergent adverse events (TEAEs), the incidence of AEs leading to discontinuation, vital signs measurements, pulse oximetry, physical examination, weight, laboratory test results, ECG findings, ACES, C-SSRS results, oral administration site findings, as well as concomitant medications.

TABLE 11
Schedule of Events for Cohorts 1-3

| | Activity | | | | | | | | | | | | |
|---|---|---|---|---|---|---|---|---|---|---|---|---|---|
| | | Night | | | | | | | | | | | |
| | | before | | | | | | | | | | | |
| | Screening | first | | | | | | | | | | | |
| | (−21 d | treat- | | | | | | | | | Post | | |
| | to −2 d) | ment | Treatment Days | | | | | | | | Treatment¹⁶ | | |
| | Time point | | | | | | | | | | | | |
| | Pre- | | Day 1: | | | | | | | | | | |
| | Treatment | Day 0 | Pre-Dose | Day 1 | Day 2 | Day 3 | Day 4 | Day 5 | Day 6 | Day 7 | Day 8 | Day 9 | Day 10 |
| Informed Consent | X | | | | | | | | | | | | |
| Review Inc/Exc | X | X | X | | | | | | | | | | |
| Criteria | | | | | | | | | | | | | |
| Medical History | X | | | | | | | | | | | | |
| Demographics | X | | | | | | | | | | | | |
| Weight | X | X | | | | | | | | | X | | X |
| Height | X | | | | | | | | | | | | |
| Physical Exam | X | X | | | | | | | | | X | | X |
| Safety Labs² | X | | X | | | | | | | | X | | X |
| Urine drug screen | X | X | | | | | | | | | | | |
| Serum Pregnancy | X | | | | | | | | | | | | |
| Test³ | | | | | | | | | | | | | |
| Urine Pregnancy | | X | | | | | | | | | | | |
| Test³ | | | | | | | | | | | | | |
| Resting vital | X | X | X | X⁸ | X⁸ | X⁸ | X⁸ | X⁸ | X⁸ | X⁸ | X | X | X |
| signs⁴ | | | | | | | | | | | | | |
| Orthostatic vital | X | X | X | X⁹ | X⁹ | X⁹ | X⁹ | X⁹ | X⁹ | X⁹ | X | X | X |
| signs⁵ | | | | | | | | | | | | | |
| ECG | X | X | X | X¹⁰ | | | X¹⁰ | X^{10,17} | X^{10,17} | X¹⁰ | X | X | X |
| C-SSRS | X | X | X | | | | | | | | X | X | X |
| ACES | X | | X | X¹¹ | | | X¹³ | | | X¹³ | X | X | X |
| MMSE | | | X | | | | | | | X¹⁴ | X | X | X |
| Concomitant | X | X | X | X | X | X | X | X | X | X | X | X | X |
| medications | | | | | | | | | | | | | |
| Adverse events | X | X | X | X | X | X | X | X | X | X | X | X | X |
| Pre-dose PK | | | X | | X | X | X | X | X | X | | | |
| sample | | | | | | | | | | | | | |
| Randomization | | | X | | | | | | | | | | |
| Study Drug | | | | X | X | X | X | X | X | X | | | |
| Administration⁶ | | | | | | | | | | | | | |
| Local Irritation | | | | X | X | X | X | X | X | X | X | X | X |
| check⁷ | | | | | | | | | | | | | |
| Frequent PK | | | | X | X | | | | | X | | | |
| Sampling¹² | | | | | | | | | | | | | |
| Fasting glucose¹⁵ | | | | | X | X | X | X | X | X | | X | |
| Pre-Dose Resting | | | | | X | X | X | X | X | X | | | |
| Vital Signs⁴ | | | | | | | | | | | | | |
| Pre-Dose | | | | | X | X | X | X | X | X | | | |
| Orthostatic Vital | | | | | | | | | | | | | |
| Signs^{5,16} | | | | | | | | | | | | | |
| Duloxetine | | | | X | X | X | X | X | X | X | | | |
| administration | | | | | | | | | | | | | |
| (Cohort 6 only)¹⁸ | | | | | | | | | | | | | |

Notes to the Schedule of Events for Cohorts 1-3:
¹If cohort 6 involves only AM dosing of dexmedetomidine sublingual film, this schedule should be followed for Cohort 6.
²To be conducted while fasting.
³For women of childbearing potential
⁴Resting vital signs: temperature, pulse oximetry, heart rate and blood pressure after resting 5 minutes in the supine position
⁵Orthostatic vital signs: immediately follows collection of resting vital signs; collected at 1 and 3 minutes after standing.
⁶Study drug administration: daily between 8:30 AM-10:00 AM.
⁷Local irritation check: performed 2 hours post-dose on Days 1-7, and once per day on Days 8-10.
⁸Resting vital signs: taken 2, 4, and 6 hours post-dose.
⁹Orthostatic vital signs: immediately follows collection of resting vital signs at 2, 4, and 6 hours post-dose.
¹⁰ECG: To be obtained 2 hours post-dose.
¹¹ACES: Administered at 4, 6, and 8 hours post-dose on Day 1.
¹²Blood samples were drawn at 0.25, 0.50, 1.00, 2.00, 3.00, 4.00, 6.00, 8.00, 10.00, 12.00, and 24.00 hours post dose on Days 1, 2 and 7 (timing Table 2. extend draws into the following day)
¹³ACES: Administered before breakfast and at 4, 6, and 8 hours post-dose on Days 4 and 7
¹⁴MMSE to be administered post-dose on Day 7
¹⁵Fasting glucose: Obtained prior to dosing on Days 2-7
¹⁶Pre-dose orthostatic vital signs: immediately follows collection of pre-dose resting vital signs.
¹⁷ECG: Only obtained on Days 5 and 6 if QT prolongation was noted on the previous day's ECG
¹⁸Duloxetine administration: Only for subjects in cohort 6. Administered BID. Morning dose should be taken with breakfast. Evening dose should be taken when subject assumes a recumbent position to sleep for the night (ideally between 8:00 PM and midnight)

TABLE 12
Schedule of Events for Cohorts 4-5¹

| | Activity | | | | | | | | | | | | |
|---|---|---|---|---|---|---|---|---|---|---|---|---|---|
| | | Night | | | | | | | | | | | |
| | | before | | | | | | | | | | | |
| | Screening | first | | | | | | | | | | | |
| | (−28 d | treat- | | | | | | | | | Post | | |
| | to −2 d) | ment | Treatment Days | | | | | | | | Treatment¹⁶ | | |
| | Time point | | | | | | | | | | | | |
| | Pre- | | Day 1: | | | | | | | | | | |
| | Treatment | Day 0 | Pre-Dose | Day 1 | Day 2 | Day 3 | Day 4 | Day 5 | Day 6 | Day 7 | Day 8 | Day 9 | Day 10 |
| Informed Consent | X | | | | | | | | | | | | |
| Review Inc/Exc | X | X | X | | | | | | | | | | |
| Criteria | | | | | | | | | | | | | |
| Medical History | X | | | | | | | | | | | | |
| Demographics | X | | | | | | | | | | | | |
| Weight | X | X | | | | | | | | | X | | X |
| Height | X | | | | | | | | | | | | |
| Physical Exam | X | X | | | | | | | | | X | | X |
| Safety Labs² | X | | X | | | | | | | | X | | X |
| Urine drug screen | X | X | | | | | | | | | | | |
| Serum Pregnancy | X | | | | | | | | | | | | |
| Test³ | | | | | | | | | | | | | |
| Urine Pregnancy | | X | | | | | | | | | | | |
| Test³ | | | | | | | | | | | | | |
| Resting vital | X | X | X | X⁸ | X⁸ | X⁸ | X⁸ | X⁸ | X⁸ | X⁸ | X | X | X |
| signs⁴ | | | | | | | | | | | | | |
| Orthostatic vital | X | X | X | X⁹ | X⁹ | X⁹ | X⁹ | X⁹ | X⁹ | X⁹ | X | X | X |
| signs⁵ | | | | | | | | | | | | | |

Notes to the Schedule of Events for Cohorts 4-5:
¹If cohort 6 involves BID dosing of dexmedetomidine sublingual film, this schedule should be followed for Cohort 6.
²To be conducted while fasting.
³For women of childbearing potential
⁴Resting vital signs: temperature, pulse oximetry, heart rate and blood pressure after resting 5 minutes in the supine position
⁵Orthostatic vital signs: immediately follows collection of resting vital signs; collected at 1 and 3 minutes after standing.
⁶Study drug administration: AM dose taken between 8:30 AM-10:00 AM. PM dose taken 8:30 PM-10:00 PM.
⁷Local irritation check: performed 2 hours post- post-AM dose on Days 1-7, and once per day on Days 8-10.
⁸Resting vital signs: taken 2-, 4-, and 6-hours post-dose (AM and PM dose).
⁹Orthostatic vital signs: immediately follows collection of resting vital signs at 2-, 4-, and 6-hours post-AM dose
¹⁰ECG: To be obtained 2 hours post-dose (AM and PM dose).
¹¹ACES: Administered at 4, 6, and 8 hours post-AM dose on Day 1.
¹²Blood samples will be drawn at 0.25, 0.50, 1.00, 2.00, 3.00, 4.00, 6.00, 8.00, 10.00, 12.00, and 24.00 hours post dose (AM and PM dose) on Days 1, 2 and 7 (timing will extend draws into the following day)
¹³ACES: Administered before breakfast and at 4-, 6-, and 8-hours post-AM dose on Days 4 and 7
¹⁴MMSE to be administered post- PM dose on Day 7
¹⁵Fasting glucose: Obtained prior to dosing on Days 2-7
¹⁶Pre-dose orthostatic vital signs: immediately follows collection of pre-dose resting vital signs.
¹⁷ECG: Only obtained on Days 5 and 6 if QT prolongation was noted on the previous day's ECG
¹⁸Duloxetine administration: Only for subjects in cohort 6. Administered BID. Morning dose should be taken with breakfast. Evening dose should be taken when subject assumes a recumbent position to sleep for the night (ideally between 8:00 PM and midnight)

### Example 4: A Phase-2 Study on the Evaluation of the Effect of Dexmedetomidine Sublingual Film in Major Depressive Episode

Primary Objective: To determine if a single dose of dexmedetomidine hydrochloride sublingual film effectively reduces symptoms associated with MDE as assessed using the Hamilton Depression Rating Scale-17 Item (HAM-D-17) as compared to placebo at the end of one week of treatment.

Key Secondary Objective: To determine if a single dose of dexmedetomidine hydrochloride sublingual film effectively reduces symptoms associated with MDD with anxious distress as assessed using the Hamilton Depression Rating Scale-17 Item (HAM-D-17) as compared to placebo at the end of 4 weeks of treatment.

Other Exploratory Objectives: To further determine the efficacy, safety, tolerability and PK of dexmedetomidine hydrochloride sublingual film in patients diagnosed with MDD, including:
To determine the overall clinical improvement after drug administration as measured by the Clinical Global Impression-Improvement Scale (CGI-I).
To determine the impact of dexmedetomidine hydrochloride sublingual film on sleep disturbance as measured by the HAM-D-17 Sleep Subscale.
To determine the impact of dexmedetomidine hydrochloride sublingual film on core symptoms of depression as measured by the HAM-D-17 Depression Subscale.
To determine the safety profile of dexmedetomidine hydrochloride sublingual film as measured by reports of adverse events and vital signs.
To describe the overall tolerability in terms of treatment-emergent adverse event reports and local site (oral/sublingual) tolerability of oral film.

Study Design: This will be a randomized, double-blind, parallel group, placebo-controlled Phase 2 study assessing efficacy, safety and tolerability of dexmedetomidine hydrochloride sublingual film dosing in adult (18-75 years old) males and females with MDE as defined by DSM-5 criteria.

This will be an outpatient study with subjects randomized 1:1 to receive dexmedetomidine hydrochloride sublingual film (120 μg dose of DEX) or matching placebo film in addition to one of four SSRIs (i.e., sertraline, fluoxetine, citalopram, escitalopram). The randomization will be stratified by age; age <65 and age ≥65.

Eligible subjects (subjects diagnosed with MDD with anxious distress) will be identified in outpatient clinics, or mental health, psychiatric or medical emergency services including medical/psychiatric observation units. Subjects will undergo screening procedures on an outpatient basis.

Upon confirmation of eligibility, subjects will be randomized to receive 120 μg dexmedetomidine sublingual film or matching placebo. Subjects will be dosed nightly with dexmedetomidine sublingual film 120 μg doses or matching placebo for 4 weeks in conjunction with the titration of one of the following SSRIs: sertraline; fluoxetine; citalopram; escitalopram.

Treatment groups will be comprised of a suitable number of subjects receiving 120 μg dexmedetomidine sublingual film+SSRI or placebo+SSRI. SSRI dosing will start at half the recommended dose for weeks 1 and 2, and will increase to the recommended dose for weeks 3 and 4.

This study will include two phases: an inpatient/in-clinic phase primarily to confirm safety while commencing dosing of dexmedetomidine sublingual film and SSRI for up to one week and an outpatient phase to assess safety, efficacy, and tolerability of sublingual dexmedetomidine on an outpatient basis.

In the event of persistent or recurrent anxiety and restlessness, investigators may choose to repeat dose at half strength. Patients will only be re-dosed if they are hemodynamically stable, not hypotensive (must be greater than 90/60 systolic/diastolic) and not bradycardic (must be greater than 60 bpm). Patients also will not be re-dosed if they are orthostatic (a drop of >20 mm Hg systolic, or 10 mm Hg diastolic) or if they are experiencing an AE that in the assessment of the PI precludes redosing. The maximum number of repeat doses per subject will be 1, during the 12 hours post first dose. Doses will not be administered sooner than 2 hours after a previous dose.

Participants will also be evaluated for local irritation around the area where the film is placed. Efficacy and safety assessments will be conducted periodically before and after dosing. All efforts will be made to have the patient perform all assessments as per protocol.

Efficacy, safety, tolerability, and pharmacokinetics (PK) will be measured throughout the treatment period at various timepoints.

Number of subjects (planned): Approximately 80 subjects. This trial will be conducted at up to 3 sites in the United States.

### Subjects:

Eligible individuals will include those diagnosed with MDE with anxious distress signed an informed consent form (ICF) before any study-related procedures are performed. Upon confirmation of eligibility, subjects will be randomized to either sublingual dexmedetomidine 120 μg film or matching placebo in a 1:1 randomization.

Test Product, Dose, and Mode of Administration: Dexmedetomidine hydrochloride will be provided as a thin film formulation of DEX for sublingual (SL) administration. Dosing will deliver 60 μg or 120 μg of DEX sublingually. The product will be a small, solid-dose film formulation, approximately 193.6 mm² in area and 0.7 mm thick, designed to completely dissolve in the sublingual space within 1-3 minutes.

Reference therapy, dosage and mode of administration: Films will be taken sublingually or buccally as described above.
Duration of Treatment: 4 weeks

The foregoing examples and description of certain embodiments should be taken as illustrating, rather than as limiting the present invention as defined by the claims. As will be readily appreciated, numerous variations and combinations of the features set forth above can be utilized without departing from the present invention as set forth in the claims. All such variations are intended to be included within the scope of the present invention. All references cited are incorporated herein by reference in their entireties.

1. A method of treating major depressive disorder or a major depressive episode in a human subject in need thereof, comprising:
(i) administering at least one daily dose of dexmedetomidine or a pharmaceutically acceptable salt thereof to the subject for a period of at least 14 days, and
(ii) administering an anti-depressant to the subject.

2. The method of claim 1, wherein dexrnedetomidine or a pharmaceutically acceptable salt thereof is administered twice daily to the subject.

3. The method of claim 2, wherein the first dose of dexmedetomidine or a pharmaceutically acceptable salt thereof is administered in the morning and second dose is administered in the evening.

4. The method of claim 2, wherein the first dose of dexmedetomidine or a pharmaceutically acceptable salt thereof is lower than the second dose of dexmedetomidine or a pharmaceutically acceptable salt thereof.

5. The method of claim 1, wherein the total daily dexmedetomidine or a pharmaceutically acceptable salt thereof dose is about 30 micrograms to about 200 micrograms.

6. The method of claim 5, wherein the first dose of dexmedetomidine is about 30 micrograms and the second dose of dexmedetomidine or a pharmaceutically acceptable salt thereof is about 60 micrograms.

7. The method of claim 5, wherein the first dose of dexmedetomidine is about 40 micrograms and the second dose of dexmedetomidine or a pharmaceutically acceptable salt thereof is about 80 micrograms.

8. The method of claim 7, wherein dexmedetomidine or a pharmaceutically acceptable salt thereof is administered through routes selected from oral, parenteral, topical, intranasal, inhalation or oromucosal.

9. The method of claim 8, wherein dexmedetomidine or a pharmaceutically acceptable salt thereof is administered oromucosally in the form of tablet, wafer, patch, liquid spray, gel, film or spray.

10. The method of claim 9, wherein dexmedetomidine or a pharmaceutically acceptable salt thereof is administered oromucosally in the form of a film.

11. The method of claim 1, wherein the subject is non-agitated at the time of administration.

12. The method of claim 1, wherein the major depressive disorder is associated with anxious distress.

13. The method of claim 1, wherein the antidepressant is administered orally for an extended period of time for at least 1 month, at least 2 months, at least 3 months, at least 4 months, at least 5 months or more till the underlying disease resolves.

14. The method of claim 1, wherein the anti-depressant is an SSRI or an SNRI.

15-18. (canceled)

19. The method of claim 14, wherein the SSRI is escitalopram or citalopram or a pharmaceutically acceptable salt thereof.

20. The method of claim 14, wherein the SNRI is selected from the group consisting of desvenlafaxine, venlafaxine, levomilnacipran, milnacipran, duloxetine, atomoxetine, sibutramine, reboxetine, tramadol or a pharmaceutically acceptable salt or ester or enantiomer thereof.

21-78. (canceled)
